SEQUENCE LISTING (22 sequences)

SEQ ID NO: 1 - DNA, 17 bp, synthetic construct
  source 1..17: mol_type = other DNA
ctaacctttcaggccag

SEQ ID NO: 2 - RNA, 17 nt, synthetic construct
  source 1..17: mol_type = other RNA
ctaacctttcaggccag

SEQ ID NO: 3 - DNA, 24 bp, synthetic construct
  source 1..24: mol_type = other DNA
gtccactaacctttcaggccagcg

SEQ ID NO: 4 - RNA, 24 nt, synthetic construct
  source 1..24: mol_type = other RNA
gtccactaacctttcaggccagcg

SEQ ID NO: 5 - protein, 33 aa, synthetic construct
YYCDYCDTYLTHDSPSVRKTHCTGRKHRDNVKF

SEQ ID NO: 6 - RNA, 11 nt, unidentified
  source 1..11: mol_type = unassigned RNA
atacttacctg

SEQ ID NO: 7 - RNA, 12 nt, unidentified
  source 1..12: mol_type = unassigned RNA
ggcctcacgttc

SEQ ID NO: 8 - DNA, 52 bp, unidentified
  source 1..52: mol_type = unassigned DNA
ctcccgtcctcgcctctgtcgactatcaggtaagcgccgcggctccgaaatc

SEQ ID NO: 9 - DNA, 64 bp, synthetic construct
  source 1..64: mol_type = other DNA
gaggcgacaggagagggcaggagcggagacagctgatagtccattcgcggcgccgaggctttag

SEQ ID NO: 10 - RNA, 165 nt, unidentified
  source 1..165: mol_type = unassigned RNA
  modified_base 72: mod_base = OTHER; note = 2'-O-methyl adenosine
  modified_base 7: mod_base = OTHER; note = 2'-O-methyl adenosine
  modified_base 6: mod_base = OTHER; note = 2'-O-methyl uridine
atacttacctggcagggggagataccatgatcacgaaggtggttttccccagggcgaggcttatccattgcactccggatgtgctgacccctgtgatttccccaaatgtgggaaactcgactgcataattgtggtagtgggggactgcgttcgcgctttcccctg

SEQ ID NO: 11 - RNA, 51 nt, Homo sapiens
  source 1..51: mol_type = genomic RNA
ccgtcctcgcctctgtcgactatcaggtaagcgccgcggctccgaaatctg

SEQ ID NO: 12 - RNA, 55 nt, unidentified
  source 1..55: mol_type = unassigned RNA
ggatccattgcactccggatccaggagataccatgatcacgaaggtggttttcct

SEQ ID NO: 13 - protein, 279 aa, unidentified
GVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTFVERDRKREKRGSGSGSGSGSGSGAETREERMERKRREKIERRQQEVETELKMWDPHNDPNAQGDAFKTLFVARVNYDTTESKLRREFEVYGPIKRIHMVYSKRSGKPRGYAFIEYEHERDMHSAYKHADGKKIDGRRVLVDVERGRTVKGWRPRRLGGGLGGTRRGGADVNIRHSGRDD

SEQ ID NO: 14 - RNA, 51 nt, unidentified; identical to SEQ ID NO: 11
  source 1..51: mol_type = unassigned RNA

SEQ ID NO: 15 - RNA, 55 nt, unidentified; identical to SEQ ID NO: 12
  source 1..55: mol_type = unassigned RNA

SEQ ID NO: 16 - protein, 279 aa, unidentified; identical to SEQ ID NO: 13

SEQ ID NO: 17 - protein, 145 aa, unidentified
MPKYYCDYCDTYLTHDSPSVRKTHCTGRKHRDNVKFYYQKWMEEQAQHLIDATTAAFKAGKITNNPFAGGPGGAPPKPAGVSIPPPNMGAPPRPGMPGMPYMPPLMNPMMGMRPPPIMNPMAMMGPPPPLGTIPGVRPGIMNGPK

SEQ ID NO: 18 - DNA, 24 bp, synthetic construct
  source 1..24: mol_type = other DNA
  modified_base 1: mod_base = OTHER; note = 2'-O-methyl Phosphorothioate Guanosine
  modified_base 2: mod_base = OTHER; note = 2'-O-methyl Phosphorothioate Thymidine
  modified_base 3: mod_base = OTHER; note = 2'-O-methyl Phosphorothioate Cytosine
  modified_base 4: mod_base = OTHER; note = 2'-O-methyl Phosphorothioate Cytosine
  modified_base 5: mod_base = OTHER; note = 2'-O-methyl Phosphorothioate (Thiol) adenosine
  modified_base 6: mod_base = OTHER; note = Phosphorothioate Cytosine
  modified_base 7: mod_base = OTHER; note = Phosphorothioate Thymidine
  modified_base 8: mod_base = OTHER; note = Phosphorothioate Adenosine
  modified_base 9: mod_base = OTHER; note = Phosphorothioate Adenosine
  modified_base 10: mod_base = OTHER; note = Phosphorothioate Cytosine
  modified_base 11: mod_base = OTHER; note = Phosphorothioate Cytosine
  modified_base 12: mod_base = OTHER; note = Phosphorothioate Thymidine
  modified_base 13: mod_base = OTHER; note = Phosphorothioate Thymidine
  modified_base 14: mod_base = OTHER; note = Phosphorothioate Thymidine
  modified_base 15: mod_base = OTHER; note = Phosphorothioate Cytosine
  modified_base 16: mod_base = OTHER; note = Phosphorothioate Adenosine
  modified_base 17: mod_base = OTHER; note = Phosphorothioate Guanosine
  modified_base 18: mod_base = OTHER; note = Phosphorothioate Guanosine
  modified_base 19: mod_base = OTHER; note = Phosphorothioate Cytosine
  modified_base 20: mod_base = OTHER; note = 2'-O-methyl Phosphorothioate Cytosine
  modified_base 21: mod_base = OTHER; note = 2'-O-methyl Phosphorothioate Adenosine
  modified_base 22: mod_base = OTHER; note = 2'-O-methyl Phosphorothioate Guanosine
  modified_base 23: mod_base = OTHER; note = 2'-O-methyl Phosphorothioate Cytosine
  modified_base 24: mod_base = OTHER; note = 2'-O-methyl Phosphorothioate Guanosine
gtccactaacctttcaggccagcg

SEQ ID NO: 19 - RNA, 20 nt, synthetic construct
  source 1..20: mol_type = other RNA
  modified_base 11: mod_base = OTHER; note = 2'-O-methyl Adenosine
tatccattgcactccggatg

SEQ ID NO: 20 - DNA, 24 bp, synthetic construct
  source 1..24: mol_type = other DNA
  modified_base 1: mod_base = OTHER; note = 2'-O-methyl Phosphorothioate Guanosine
  modified_base 2: mod_base = OTHER; note = 2'-O-methyl Phosphorothioate Thymidine
  modified_base 3: mod_base = OTHER; note = 2'-O-methyl Phosphorothioate Cytosine
  modified_base 4: mod_base = OTHER; note = 2'-O-methyl Phosphorothioate (Thiol) cytosine
  modified_base 5: mod_base = OTHER; note = 2'-O-methyl Phosphorothioate Adenosine
  modified_base 6: mod_base = OTHER; note = Phosphorothioate Cytosine
  modified_base 7: mod_base = OTHER; note = Phosphorothioate Thymidine
  modified_base 8: mod_base = OTHER; note = Phosphorothioate Adenosine
  modified_base 9: mod_base = OTHER; note = Phosphorothioate Adenosine
  modified_base 10: mod_base = OTHER; note = Phosphorothioate Cytosine
  modified_base 11: mod_base = OTHER; note = Phosphorothioate Cytosine
  modified_base 12: mod_base = OTHER; note = Phosphorothioate Thymidine
  modified_base 13: mod_base = OTHER; note = Phosphorothioate Thymidine
  modified_base 14: mod_base = OTHER; note = Phosphorothioate Thymidine
  modified_base 15: mod_base = OTHER; note = Phosphorothioate Cytosine
  modified_base 16: mod_base = OTHER; note = Phosphorothioate Adenosine
  modified_base 17: mod_base = OTHER; note = Phosphorothioate Guanosine
  modified_base 18: mod_base = OTHER; note = Phosphorothioate Guanosine
  modified_base 19: mod_base = OTHER; note = Phosphorothioate Cytosine
  modified_base 20: mod_base = OTHER; note = 2'-O-methyl Phosphorothioate Cytosine
  modified_base 21: mod_base = OTHER; note = 2'-O-methyl Phosphorothioate Adenosine
  modified_base 22: mod_base = OTHER; note = 2'-O-methyl Phosphorothioate Guanosine
  modified_base 23: mod_base = OTHER; note = 2'-O-methyl Cytosine
  modified_base 24: mod_base = OTHER; note = 2'-O-methyl Guanosine
gtccactaacctttcaggccagcg

SEQ ID NO: 21 - RNA, 16 nt, synthetic construct
  source 1..16: mol_type = other RNA
  misc_difference 11: note = a, c, u or g
yyyyyyyyyyncaggr

SEQ ID NO: 22 - RNA, 20 nt, synthetic construct
  source 1..20: mol_type = other RNA
gtaggcctcacgttacctat

(19) United States
(12) Patent Application Publication
(10) Pub. No.: US 2026/0085315 A1
(43) Pub. Date: Mar. 26, 2026

(54) POLYNUCLEOTIDE COMPOSITIONS AND METHODS FOR TREATMENT OF CANCER

(71) Applicant: APTAH BIO, INC., San Carlos, CA (US)

(72) Inventor: Caio BRUNO QUINTA DE SOUZA LEAL, Florianópolis - SC (BR)

(21) Appl. No.: 19/272,428

(22) Filed: Jul. 17, 2025

Related U.S. Application Data
(63) Continuation of application No. PCT/US2024/011891, filed on Jan. 17, 2024.
(60) Provisional application No. 63/480,466, filed on Jan. 18, 2023.

(51) Int. Cl. C12N 15/113; A61P 35/00

(52) U.S. Cl. CPC C12N 15/113; A61P 35/00; C12N 2310/315; C12N 2310/321; C12N 2310/322

(57) ABSTRACT

This disclosure provides an engineered polynucleotide that interacts with a pre-mRNA and a spliceosome to regulate gene expression. The engineered polynucleotide may have stem-loop structure that recruits the spliceosome and targeting sequences that are complementary to a target sequence at an exon-intron splice junction and may include nucleotides with 2′ modifications and phosphorothioate linkages. The engineered polynucleotide can be administered to a subject to treat a cancer.

### CROSS REFERENCE

This application is a continuation application of International Application No. PCT/US2024/011891, filed Jan. 17, 2024, which claims the benefit of U.S. Provisional Application No. 63/480,466, filed Jan. 18, 2023, which applications are incorporated herein by reference in their entirety.

### SEQUENCE LISTING

The instant application contains a Sequence Listing which has been submitted electronically in XML format and is hereby incorporated by reference in its entirety. Said XML copy, created on Dec. 8, 2025, is named 61572-704_301.xml and is 50,711 bytes in size.

### BACKGROUND

Aberrant splicing has been implicated in many disease states. Proper splicing of pre-messenger RNA (pre-mRNA) is important for proper translation of proteins. Efficiency of splicing must be regulated in order to ensure proper splicing of pre-mRNA as well as proper premature polyadenylation suppression to avoid the production of misfolded and possibly disease-causing proteins. Efficiency of gene regulations at ribonucleic acid (RNA) levels remain limited. Accordingly, there is a need for developing polynucleotide compositions and methods for regulating gene expression and activity, e.g., at therapeutically efficacious and safe levels.

### SUMMARY

Described herein, in some aspects, is a method of treating cancer in a subject in need thereof comprising administering to the subject a pharmaceutical composition comprising an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

Described herein, in some aspects, is a method of treating cancer in subject in need thereof comprising: administering to the subject a pharmaceutical composition comprising (a) a taxane and (b) an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the method improves the prognosis of the subject compared to a subject that is administered the taxane and is not administered engineered polynucleotides. In some embodiments, the method reduces the total level of tau in a subject, thereby decreasing the amount of tau that is bound to the taxane.

Described herein, in some aspects, is a method of improving the prognosis of a subject suffering from cancer and has been administered a taxane, wherein the method comprises: administering to the subject a pharmaceutical composition comprising an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the method reduces the total level of tau in a subject, thereby decreasing the amount of tau that is bound to taxane in a subject.

In some embodiments, the cancer is selected from the group consisting of: brain cancer, prostate cancer, breast cancer, renal cancer, kidney cancer, lung cancer, and liver cancer. In some embodiments, the cancer is selected from the group consisting of: glioblastoma (GBM), neuroblastoma, hepatocellular carcinoma, breast adenocarcinoma, human prostate adenocarcinoma, renal cell carcinoma, and kidney adenocarcinoma. In some embodiments, the cancer is a GBM.

In some embodiments, the methods disclosed herein decreases premature polyadenylation of one or more transcripts of the subject. In some embodiments, the methods disclosed herein decreases cryptic splicing of one or more transcripts of the subject. In some embodiments, the methods disclosed herein improve a score associated with a histopathological finding. In some embodiments, the histopathological finding comprises tumor grade, lipid content, necrosis, or nucleus-to-cytoplasmic (N:C) ratio.

In some embodiments, the method reduces the tumor volume ratio. In some embodiments, the method reduces the tumor progression. In some embodiments, the method alters the expression of tau. In some embodiments, the method reduces the expression of tau. In some embodiments, the method reduces the total amount of tau in the subject. In some embodiments, the method reduces the expression of Akt. In some embodiments, the method reduces the expression of glial fibrillary acidic protein (GFAP). In some embodiments, the engineered polynucleotide is administered intratumorally. In some embodiments, the engineered polynucleotide is administered intravenously. In some embodiments, the engineered polynucleotide is administered intrathecally. In some embodiments the engineered polynucleotide is administered via subcutaneous injection, intramuscular injection, intradermal injection, transdermal injection percutaneous administration, intranasal administration, intralymphatic injection, intrathecal administration, pulmonary administration, rectal administration intragastric administration, or any other suitable parenteral administration.

Described herein, in some aspects, is a method of reducing the viability of a cell, the method comprising: administering to a cell an engineered polynucleotide comprising: (i) one or more targeting moieties configured to specifically bind a pre-messenger ribonucleic acid; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

Described herein, in some aspects, is a method of decreasing the proliferation rate of a cell, the method comprising: administering to a cell an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

In some embodiments, the method increases cell necrosis or apoptosis. In some embodiments, the method increases the propensity of the cell to be in a G2/M phase. In some embodiments, the cell is a tumor cell. In some embodiments, the tumor cell is a glioma, neuroblastoma, or carcinoma.

Described herein, in some aspects, is a method of altering the distribution of cell cycles phases of a plurality of cells, the method comprising: administering to the plurality of cells an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

In some embodiments, the method increases the number of cells that are in a G2/M phase. In some embodiments, the method increases the number of cells in a necrotic or apoptotic phase. In some embodiments, the plurality of cells comprises a tumor. In some embodiments, the tumor cells comprise a glioma, neuroblastoma, or carcinoma.

Described herein, in some aspects, is a method of reducing tau expression by a neuron, the method comprising: administering to the neuron an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the neuron is from an individual afflicted with a cancer. In some embodiments, the method decreases cryptic splicing of one or more transcripts of the neuron.

Described herein, in some aspects, is a method of treating a subject with cancer, comprising administering to the subject a pharmaceutical composition comprising an engineered polynucleotide having the nucleotide sequence of SEQ ID NO: 3, wherein all internucleotide bonds of the engineered polynucleotide comprise a phosphorothioate linkage and wherein nucleotides at positions 1-5 and 20-24 of the engineered polynucleotide comprise a 2′-O-methyl moiety, thereby modulating U1 snRNP complex formation.

Described herein, in some aspects, is a method of treating a subject with cancer, comprising administering to the subject a pharmaceutical composition comprising an engineered polynucleotide having the nucleotide sequence of SEQ ID NO: 3. In some embodiments the internucleotide bonds of the engineered polynucleotide comprise a phosphorothioate linkage, In some embodiments, the nucleotides at positions 1-5 and 20-24 of the engineered polynucleotide comprise a 2′-O-methyl moiety. In some embodiments, the method further comprises administering a taxane to the subject.

Described herein, in some aspects, is a method of treating a subject with cancer, wherein the subject has been administered a taxane, the method comprising administering to the subject a pharmaceutical composition comprising (i) an engineered polynucleotide having the nucleotide sequence of SEQ ID NO: 3, wherein internucleotide bonds of the engineered polynucleotide comprise a phosphorothioate linkage and wherein nucleotides at positions 1-5 and 20-24 of the engineered polynucleotide comprise a 2′-O-methyl moiety, thereby reducing tau protein levels and tau binding to the taxane in the subject.

In some embodiments, the methods disclosed herein modulate the formation of U1 snRNP complexes.

Described herein, in some aspects, are engineered polynucleotides for use in the methods disclosed throughout this disclosure. In some embodiments, the engineered polynucleotide comprises a nucleotide sequence that is at least 70%, 80%, 85%, or 90% identical or complementary to any one of SEQ ID NOs: 1-4. In some embodiments, the engineered polynucleotide comprises a nucleotide sequence that is identical or complementary to any one of SEQ ID NOs: 3 or 4.

In some embodiments, the engineered polynucleotide comprises: one or more targeting moiety configured to specifically bind a pre-messenger ribonucleic acid (pre-mRNA) at a target sequence therein; and a recruiting moiety configured to recruit a post-transcriptional regulating moiety (e.g., a spliceosomal moiety), wherein, when associated with the pre-mRNA and the engineered polynucleotide, the post-transcriptional regulating moiety alters the pre-mRNA in or in proximity to the target sequence. In some embodiments, a targeting moiety of the one or more targeting moiety is sufficiently identical or complementary to a consensus sequence in the target sequence of a target gene. In some embodiments, a targeting moiety is complementary to and/or hybridizes with the target sequence. In some embodiments, a targeting moiety is complementary to and/or hybridizes with the consensus sequence of the target sequence. In some embodiment, the target sequence comprises a splice site. In some embodiment, splice sites is a conserved splice site. In some embodiment, splice site comprises 5′-GU-3′ In some embodiment, pre-mRNA is encoded by the target gene In some embodiment, the method alters an expression or activity of the target gene.

In some embodiments, the one or more targeting moiety comprises a first targeting moiety configured to specifically bind a first targeted sequence in the target sequence of the pre-mRNA, and a second targeting moiety configured to specifically bind a second targeted sequence in the target sequence of the pre-mRNA. In some embodiments, the first targeted sequence comprises a consensus sequence in the target sequence. In some embodiments, the second targeted sequence comprises a consensus sequence in the target sequence. In some embodiments, the first and second targeted sequences are apart in the target sequence by a spacing sequence of no more than five nucleotides (e.g., one or two nucleotides). In some embodiments, the target sequence comprises an exon-intron boundary in the pre-mRNA. In some embodiments, the first and second targeted sequences are both 5′ or 3′ with respect to the exon-intron boundary. In some embodiments, one of the first and second targeted sequences is 5′ with respect to the exon-intron boundary; and wherein the other of the first and second targeted sequences is 3′ with respect to the exon-intron boundary. In some embodiments, the target sequence comprises a splice site in the pre-mRNA. In some embodiments, the first or second targeted sequence comprises a splice site (e.g., 5′ ss) in the pre-mRNA. In some embodiments, one of the first and second targeting moieties is 5′ with respect to the recruiting moiety, and the other of the first and second targeting moieties is 3′ with respect to the recruiting moiety. In some embodiments, the targeting moiety comprises a sequence at least 80%, 90%, or identical to a ribosome binding site of a spliceosome snRNA, e.g., U1 snRNA. In some embodiments, the first targeting moiety comprises a sequence at least 80%, 90%, or identical to a ribosome binding site of a spliceosome snRNA, e.g., U1 snRNA. In some embodiments, the second targeting moiety comprises a sequence at least 80%, 90%, or identical to a ribosome binding site of a spliceosome snRNA, e.g., U1 snRNA. The sequence at least 80%, 90%, or identical to the ribosome binding site may be about 2 nucleotides to about 10 nucleotides. In some embodiments, the recruiting nucleotide sequence comprising: (i) a nucleotide sequence complementary to at least 4 nucleotides of the Stem-Loop II (SL2) of an U1 snRNA.

In some embodiments, the first targeting moiety or the second targeting moiety comprises a sequence identical or complementary to a sequence set forth in Table 1. In some embodiments, the first targeting moiety comprises a sequence identical or complementary to a sequence selected from the 5′-targeting moiety sequences column of Table 1; and wherein the second targeting moiety comprises a sequence identical or complementary to a sequence set forth in the 3′-targeting moiety sequences column of Table 1. In some embodiments, the first targeting moiety comprises a sequence identical or complementary to a sequence set forth in the 3′-targeting moiety sequences column of Table 1; and wherein the second targeting moiety comprises a sequence identical or complementary to a sequence set forth in the 5′-targeting moiety sequences column of Table 1. In some embodiments, the first targeting moiety or the second targeting moiety comprises a sequence identical or complementary to a consensus sequence of an intron donor site (e.g., selected from GU, GT, GC, and CA). In some embodiments, the first targeting moiety or the second targeting moiety comprises a sequence identical or complementary to a consensus sequence of an exon donor site (e.g., G). In some embodiments, the first targeting moiety or the second targeting moiety comprises a sequence identical or complementary to a consensus sequence selected from GU, GC, G, and CA. In some embodiments, the spliceosomal moiety is selected from a spliceosomal ribonucleoprotein complex, a spliceosomal small nuclear ribonucleic acid (snRNA), a spliceosomal protein, a functional variant thereof, or a functional fragment thereof. In some embodiments, the spliceosomal moiety comprises U1 snRNA and a spliceosomal protein. In some embodiments, the spliceosomal snRNA is selected from U1, U2, U4, U5, U6, U11, U12, U14atac, U6atac, and combinations thereof. In some embodiments, the spliceosomal protein is selected from Sm, U1-70k, U1A, U1C, and combinations thereof. In some embodiments, the recruiting moiety comprises a nucleotide sequence that is at least 70%, 80%, 85%, or 90% identical or complementary to a sequence set forth in Tables 2-3. In some embodiments, the recruiting moiety is complementary to Stem loop-II region of snRNA, e.g., U1 snRNA. In some embodiments, the recruiting moiety hybridizes with Stem loop-II region of snRNA, e.g., U1 snRNA. In some embodiments, the recruiting moiety comprises AGGCC. In some embodiments, the recruiting moiety comprises a nucleotide sequence that is at least 80%, 90%, or identical to at least five contiguous nucleotides of a sequence set forth in Tables 2-3. In some embodiments, the recruiting moiety comprises a nucleotide sequence that is at least 80%, 90%, or identical to about 5 to about 10 contiguous nucleotides of a sequence set forth in Tables 2-3. In some embodiments, the recruiting moiety comprises a nucleotide sequence that is identical or complementary to a sequence set forth in Tables 2-3. In some embodiments, the engineered polynucleotide comprises a (e.g., secondary) structural feature. In some embodiments, the engineered polynucleotide comprises an apical loop, an upper stem, an internal loop, a lower stem, or a combination thereof. In some embodiments, the engineered polynucleotide comprises a loop (e.g., an internal loop) adjacent to a stem (e.g., a lower stem or an upper stem) comprising two complementary stem sequences. In some embodiments, a stem sequence of the stem (e.g., the lower stem or the upper stem) comprises no more than about five, four, or three nucleotides. In some embodiments, the loop is an internal loop adjacent to the stem (e.g., the lower stem) and a further stem (e.g., an upper stem) comprising two complementary stem sequences. In some embodiments, the internal loop comprises a nucleic acid sequence of no more than 10, 9, or 8 nucleotides. In some embodiments, a stem sequence of the further stem (e.g., the upper stem) comprises no more than about five, four, or three nucleotides. In some embodiments, the engineered polynucleotide further comprises an apical loop. In some embodiments, the apical loop comprises a nucleic acid sequence of no more than 10, 9, 8, 7, 6, or 5 nucleotides. In some embodiments, the engineered polynucleotide does not comprise any intramolecular disulfide bond. In some embodiments, the engineered polynucleotide, when associated with the spliceosomal moiety, the pre-mRNA exhibits substantially no base pairing with an RNA binding domain (RBD) of U1 snRNA. In some embodiments, the engineered polynucleotide, when associated with the spliceosomal moiety, the pre-mRNA exhibits substantially no base-specific interaction with U1-C protein. In some embodiments, the engineered polynucleotide is configured to specifically interact with zinc-finger of U1-C protein. In some embodiments, a 5′-targeting moiety of the engineered polynucleotide is configured to specifically interact with zinc-finger of U1-C protein. In some embodiments, the engineered polynucleotide (is configured to covalently interact (e.g., via disulfide bonding) with zinc-finger of U1-C protein. In some embodiments, the engineered polynucleotide is configured to non-covalently interact (e.g., via hydrogen bonding) with zinc-finger of U1-C protein. In some embodiments, the engineered polynucleotide comprises a nucleotide sequence complementary to a partial sequence of Stem-Loop II (SL2) of U1 snRNA. In some embodiments, a side of a stem-loop structure of the engineered polynucleotide comprises a nucleotide sequence complementary to a partial sequence of Stem-Loop II (SL2) of U1 snRNA. In some embodiments, the partial sequence comprises the sequence corresponding to 5′-GGCCU-3′ of SL2 of U1 snRNA. In some embodiments, the partial sequence does not comprise the sequence corresponding to 5′-CACGUUA-3′ of SL2 of U1 snRNA. In some embodiments, the engineered polynucleotide exhibits substantially no base pairing with an anchoring sequence of SL2 of U1 snRNA. In some embodiments, an internal loop of the engineered polynucleotide exhibits substantially no base pairing with the anchoring sequence of the SL2 of U1 snRNA. In some embodiments, a lower stem of the engineered polynucleotide exhibits substantially no base pairing with the anchoring sequence of the SL2 of U1 snRNA. In some embodiments, the anchoring sequence comprises the sequence corresponding to 5′-CACGUUA-3′. In some embodiments, the engineered polynucleotide exhibits substantially no base pairing with H helix of U1 snRNA. In some embodiments, the engineered polynucleotide comprises at least one chemical modification. In some embodiments, the engineered polynucleotide comprises at least one 2′-modified (e.g., 2′-methoxy, 2′-methoxymethyl, or 2′-methoxyethyl) nucleotides. In some embodiments, at least about 50%, 60%, 70%, 80%, or 90% nucleotides of the engineered polynucleotide are chemically modified nucleotides. In some embodiments, at least about 50%, 60%, 70%, 80%, or 90% nucleotides of the engineered polynucleotide are 2′-modified (e.g., 2′-methoxy, 2′-methoxymethyl, or 2′-methoxyethyl) nucleotides. In some embodiments, the 2′-modified nucleotide comprises a 2′-methoxy, 2′-methoxymethyl, 2′-methoxyethyl, 2′ fluoro, or 2′-aminoethyl nucleotide. In some embodiments, the engineered polynucleotide comprises nucleotides connected by internucleotide linkages and at least one of the internucleotide linkages does not comprise a phosphate. In some embodiments, engineered polynucleotide comprises nucleotides connected by internucleotide linkages, and at least one of the internucleotide linkages comprises a sulfur (S); selenium (Se); BR3, wherein each R is independently selected from the group consisting of hydrogen, alkyl, and aryl; Carbon (C); or NR2, wherein each R is independently selected from the group consisting of a hydrogen, alkyl, and aryl.

In some embodiments, the engineered polynucleotide comprises at least one phosphorothioate internucleotide bond. In some embodiments, at least about 50%, 60%, 70%, 80%, or 90% internucleotide linkages of the engineered polynucleotide are chemically modified. In some embodiments, at least about 50%, 60%, 70%, 80%, or 90% internucleotide linkages are phosphorathioate. In some embodiments, the internucleotide linkages comprises a methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino. In some embodiments, the engineered polynucleotide is about 10 to about 40 nucleotides, about 10 to about 35 nucleotides, about 10 to about 30 nucleotides, or about 10 to about 25 nucleotides. In some embodiments, the recruiting moiety comprises about 10 to about 30 nucleotides, or about 10 to about 20 nucleotides. In some embodiments, the one or more targeting moiety each independently comprises about 2 to about 15 nucleotides, about 2 nucleotides to about 10 nucleotides, or about 2 nucleotides to about 8 nucleotides. In some embodiments, one of the first and second targeting moieties comprises about 2 nucleotides, and the other of the first and second targeting moieties comprises about 5 or 6 nucleotides. In some embodiments, when associated with the engineered polynucleotide and the pre-mRNA, the spliceosomal moiety cleaves or splices the pre-mRNA in the target sequence. In some embodiments, the spliceosomal moiety further facilitates modification of a cleaved pre-mRNA.

Described herein, in some aspects, is an engineered polynucleotide comprising a nucleotide sequence that is at least 70%, 80%, 85%, or 90% identical or complementary to a sequence set forth in Tables 2-3, which engineered polynucleotide is characterized by a (e.g., secondary) structural feature. In some embodiments, the nucleotide sequence is identical or complementary to a sequence set forth in Tables 2-3. In some embodiments, the structural feature comprises one or more stem-loop structures. In some embodiments, the structural feature comprises an apical loop, an upper stem, an internal loop, a lower stem, or a combination thereof. In some embodiments, the engineered polynucleotide comprises a loop (e.g., an internal loop) adjacent to a stem (e.g., a lower stem or an upper stem) comprising two complementary stem sequences. In some embodiments, a stem sequence of the stem (e.g., the lower stem or the upper stem) comprises no more than about five, four, or three nucleotides. In some embodiments, the loop is an internal loop adjacent to the stem (e.g., the lower stem) and a further stem (e.g., an upper stem) comprising two complementary stem sequences. In some embodiments, the internal loop comprises a nucleic acid sequence of no more than 10, 9, or 8 nucleotides. In some embodiments, a stem sequence of the further stem (e.g., the upper stem) comprises no more than about five, four, or three nucleotides. In some embodiments, the engineered polynucleotide further comprises an apical loop. In some embodiments, the apical loop comprises a nucleic acid sequence of no more than 10, 9, 8, 7, 6, or 5 nucleotides. In some embodiments, the engineered polynucleotide further comprises one or more targeting moiety sufficiently identical or complementary to a target sequence of a target gene. In some embodiments, a targeting moiety of the one or more targeting moiety is sufficiently identical or complementary to a consensus sequence in the target sequence of the target gene. In some embodiments, the target gene is microtubule associated protein tau (MAPT). In some embodiments, the engineered polynucleotide comprises at least one chemical modification. In some embodiments, the engineered polynucleotide comprises at least one phosphorothioate internucleotide bond. In some embodiments, at least about 50%, 60%, 70%, 80%, or 90% internucleotide linkages of the engineered polynucleotide are chemically modified. In some embodiments, at least about 50%, 60%, 70%, 80%, or 90% internucleotide linkages are phosphorathioate. In some embodiments, the engineered polynucleotide comprises at least one 2′-modified (e.g., 2′-methoxy, 2′-methoxymethyl, or 2′-methoxyethyl) nucleotides. In some embodiments, at least about 50%, 60%, 70%, 80%, or 90% nucleotides of the engineered polynucleotide are chemically modified nucleotides. In some embodiments, at least about 50%, 60%, 70%, 80%, or 90% nucleotides of the engineered polynucleotide are 2′-modified (e.g., 2′-methoxy, 2′-methoxymethyl, or 2′-methoxyethyl) nucleotides. In some embodiments, the engineered polynucleotide comprises about 10 to about 40 nucleotides, about 10 to about 35 nucleotides, about 10 to about 30 nucleotides, or about 10 to about 25 nucleotides.

Described herein, in some aspects, is a method for altering a pre-messenger ribonucleic acid (pre-mRNA) in a cell, the method comprising contacting the cell with an engineered polynucleotide that comprises one or more targeting moiety and a recruiting moiety, wherein the one or more targeting moiety binds to the pre-mRNA at a target sequence therein, and the recruiting moiety recruits a post-transcriptional regulating moiety (e.g., a spliceosomal moiety) within proximity of the target sequence of the pre-mRNA to alter the pre-mRNA in the cell, thereby yielding one or more altered pre-mRNA. In some embodiments, a targeting moiety of the one or more targeting moiety is sufficiently identical or complementary to a consensus sequence in the target sequence of a gene (e.g., a target gene). In some embodiments, the pre-mRNA corresponds to a target gene. In some embodiments, the target gene is microtubule associated protein tau (MAPT). In some embodiments, the method alters an expression or activity of the target gene. In some embodiments, prior to the contacting, the cell exhibits an aberrant messenger ribonucleic acid (mRNA) or protein corresponding to the target gene.

In some embodiments, the engineered polynucleotide comprises: (i) a first targeting moiety configured to specifically bind a pre-messenger ribonucleic acid (pre-mRNA) at a first targeted sequence therein, wherein the first targeting moiety comprises a sequence identical or complementary to 5′-GTCCA-3′, (ii) a recruiting moiety comprising a sequence that is at least 90% similar or complementary to SEQ ID NO. 1 and is configured to recruit a spliceosomal moiety that comprises U1 snRNA and a U1-C protein, wherein the recruiting moiety comprises an apical loop, an upper stem adjacent to the apical loop, a lower stem, and an internal loop situated between the upper stem and the lower stem, and (iii) a second targeting moiety configured to specifically bind the pre-mRNA at a second targeted sequence therein, wherein the second targeting moiety comprises a sequence identical or complementary to 5′-CG-3′.

Described herein, in some aspects, is a set of engineered polynucleotides each independently comprise: one or more targeting moiety configured to bind a pre-messenger ribonucleic acid (pre-mRNA) at a target sequence; and a recruiting moiety configured to recruit a post-transcriptional regulating moiety (e.g., a spliceosomal moiety), wherein the set of engineered polynucleotides are configured to specifically bind the pre-mRNA at a plurality of target sequences comprising the target sequence.

Another embodiment described herein is an engineered polynucleotide comprising a first targeting moiety configured to specifically bind a pre-messenger ribonucleic acid (pre-mRNA) at a first targeted sequence therein, a recruiting moiety configured to recruit a spliceosomal moiety, and a second targeting moiety configured to specifically bind the pre-mRNA at a second targeted sequence therein; wherein the recruiting moiety comprises an apical loop, an upper stem adjacent to the apical loop, a lower stem, and an internal loop situated between the upper stem and the lower stem, and wherein the spliceosomal moiety alters the pre-mRNA in a target sequence comprising the first targeted sequence and the second targeted sequence when associated with the pre-mRNA and the engineered polynucleotide. In some embodiments, the first targeting moiety is complementary to and/or hybridizes with the first target sequence. In some embodiments, the second targeting moiety is complementary to and/or hybridizes with the second target sequence. In some aspects, the first targeted sequence and second targeted sequences are separated in the target sequence by a spacing sequence of no more than five nucleotides. In some aspects, the target sequence comprises an exon-intron boundary in the pre-mRNA. In some aspects, the first targeted sequences is 5′ of the exon-intron boundary and the second targeted sequence is 3′ of the exon-intron boundary. In some aspects, the first targeting moiety comprises a sequence identical to or complementary to a sequence set forth in the exon sequence column of Table 1; and the second targeting moiety comprises a sequence identical to or complementary to a sequence set forth in the intron sequence column of Table 1. In some embodiments, the first targeting moiety comprises a sequence at least 80%, 90%, or identical to a ribosome binding site of a spliceosome snRNA, e.g., U1 snRNA. In some embodiments, the second targeting moiety comprises a sequence at least 80%, 90%, or identical to a ribosome binding site of a spliceosome snRNA, e.g., U1 snRNA. In some embodiments, the sequence at least 80%, 90%, or identical to the ribosome binding site may be about 2 nucleotides to about 10 nucleotides. In some embodiments, the recruiting moiety is complementary to Stem loop-II region of snRNA, e.g., U1 snRNA. In some embodiments, the recruiting moiety hybridizes with Stem loop-II region of snRNA, e.g., U1 snRNA. In some embodiments, the recruiting moiety comprises AGGCC. In some embodiments, the recruiting moiety comprises a nucleotide sequence that is at least 80%, 90%, or identical to at least five contiguous nucleotides of a sequence set forth in Tables 2-3. In some embodiments, the recruiting moiety comprises a nucleotide sequence that is at least 80%, 90%, or identical to about 5 to about 10 contiguous nucleotides of a sequence set forth in Tables 2-3. In some aspects, the spliceosomal moiety comprises a U1 snRNA and a U1-C protein. In some aspects, the upper stem or the lower stem comprises two complementary sequences and each of the two complementary sequences comprises no more than 5 nucleotides; the internal loop comprises two nucleic acid sequences and each of the two nucleic acid sequence comprises no more than 5 nucleotides; and the apical loop comprises a nucleic acid sequence of no more than 8 nucleotides. In other aspects the pre-mRNA exhibits substantially no base pairing with an RNA binding domain (RBD) of U1 snRNA when associated with the engineered polynucleotide and the spliceosomal moiety exhibits substantially no base-specific interaction with a U1-C protein when associated with the engineered polynucleotide and the spliceosomal moiety. In another aspect, a 5′-targeting moiety of the engineered polynucleotide is configured to specifically interact with a zinc-finger of U1-C protein. In another aspect, the recruiting moiety comprises a nucleotide sequence complementary to at least 4 nucleotides of a sequence of Stem-Loop II (SL2) of U1 snRNA. In another aspect, sequence of SL2 of U1 snRNA comprises 5′-GGCCU-3′. The engineered polynucleotide nay have a 2′-modified nucleotide. At least 50% of the nucleotides of the engineered polynucleotide may be 2′-modified nucleotides. The 2′-modified nucleotides may be 2′-methoxy nucleotides. In another aspect, the engineered polynucleotide comprises nucleotides connected by internucleotide linkages and at least one of the internucleotide linkages does not comprise a phosphate. At least one or 50%, 60%, 70% 80% or 90% of the internucleotide linkages may be a phosphorothioate.

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIG. 1 illustrates a schematic diagram for identification of splice-donor and splice-acceptor sites. An example consensus sequence for messenger ribonucleic acid (mRNA) splicing in animals and plants is “GU_AG,” where “GU” is an example splice donor sequence and “AG” is an example splice acceptor sequence. A longer splice donor consensus sequence in mammals may be “GUrAGU,” where “r” represents either “G” or “A.” Usually, an expression of “GU_AG” means that only the 5′ and 3′ terminal two nucleotides of the sequence are invariable as “GU” and “AG,” respectively, and that a sequence represented by the underscore can be any sequences. However, described herein this expression indicates that the sequence represented by the underscore can be any sequences except for sequences that do not match any of the other consensus sequences. The splice acceptor consensus sequence is preceded by a branch point sequence, which contains an adenine, which is ligated to the 5′ splice site ribonucleotide to form the intron lariat, and a polypyrimidine tract (C or U), which is between the branch point and the splice acceptor sequence. While the short GU_AG consensus sequence of introns is clearly not sufficient to differentiate amongst the multitude of alternative splicing events, surprisingly little is known about what other sequence information is required to regulate alternative RNA splicing. The flanking one or two nucleotides on either side of the intron are also often conserved, and they are included in our supplementary tables, but they will not be discussed further in this paper so that we can focus our analyses on consensus sequences at the ends of the introns. In this sense, the rational design of an engineered polynucleotide logically identifies the splice's intronic consensus sequences (GU_AG). Then, making it possible to determine conserved regions of the donor site (5′ exon and intron downstream) and acceptor (3′ exon and intron downstream). It is important to note that the conserved and consensus regions are located within the same site of constitutive splice donor or acceptor. The recognition of the consensus regions determines a 5′ splicing site, that is, the limit of junction between the exon and intron. Meanwhile, the recognition of the conserved regions identifies the identity of the transcript chosen for modulation.

FIGS. 2A-2B illustrate an example engineered polynucleotide described herein comprising: (1) a 3′-targeting moiety: 3′-GC-5′; (2) a lower stem: 3′-GA-5′/5′-CT3′; (3) an internal loop: 3′-CC-5′/5′-AA-3′; (4) an upper stem: 3′-GGA-5′/5′-CCT-3′; (5) an apical loop: 3′-CTT-5′; and (6) a 5′-targeting moiety: 5′-GTCCA-3′. FIG. 2C illustrates interactions of the example engineered polynucleotide with a target pre-mRNA sequence. FIG. 2D illustrates interactions of the example engineered polynucleotide with various components of a U1 RNP complex.

FIGS. 3A-3B illustrate the anchoring taking place through the engineered polynucleotide “Stem 5′/3′” (a.k.a., 5′-targeting moiety or/and 3′-targeting moiety), which is designed to interact with conserved moieties present in the constitutive donor site. FIG. 3A. Stem 5′/3′ (GTCCA and CG) such as phosphorothioate internucleotide bonds and substitutions of 2′ O-methyl (2′ O-ME) for molecular sugar increases resistance to endonucleases and increases molecular strength of interaction between the bases of the Stem 5′/3′ with conserved regions from constitutive donor. FIG. 3B. Interaction of an engineered polynucleotide described herein with constitutive donor splicing and silencing of the RNA binding moiety (RBD) of U1 snRNA with constitutive donor splicing—exon.

FIG. 4 illustrates a diagram of a human U1 snRNP. U1 snRNP is composed of one U1 snRNA, seven common Sm proteins and three U1 snRNP-specific proteins (U1-70K, U1A and U1C). Secondary structure of U1 snRNA consists of four stem-loops (SL) and an H helix highlighted. Nucleotides forming H helix are shown. In addition, U1 snRNA sequences relevant to RNA:protein or RNA:5′ss interactions are given as well. The loop portion of SL1 is drawn according to crystal structure. It is closed by a trans WC/Hoogsteen base pair formed between A29 and A36. Protein components of U1 snRNP, their sizes and their approximate locations are shown as well. The Sm ring formed by the Sm proteins shown as green circles bind to the Sm site, which is boxed. U1-70K shown in red recognizes SL1. U1A shown in yellow binds SL2. U1C shown in blue is recruited to U1 snRNP through protein:protein interactions with U1-70 K and Sm proteins. Note signifies interactions between U1C and Sm ring.

FIG. 5A-5C illustrates U1 snRNP binding to the 5′ exon-intron junction of pre-mRNA and thus playing a crucial role at an early stage of pre-mRNA splicing. Two crystal structures of engineered U1 sub-structures are shown, which together reveal at atomic resolution an almost complete network of protein-protein and RNA-protein interactions within U1 snRNP and show how the 5′ splice site of pre-mRNA is recognized by U1 snRNP. The zinc-finger of U1-C interacts with the duplex between pre-mRNA and the 5′-end of U1 snRNA. The binding of the RNA duplex is stabilized by hydrogen bonds and electrostatic interactions between U1-C and the RNA backbone around the splice junction, but U1-C makes no base-specific contacts with pre-mRNA. The structure, together with RNA binding assays, shows that the selection of 5′-splice site nucleotides by U1 snRNP is achieved predominantly through base pairing with U1 snRNA whilst U1-C fine-tunes relative affinities of mismatched 5′-splice sites. FIG. 5A. U1-70K in complex with U1 snRNA stem-loops and U1-A RRM in complex with stem-loop 2. FIG. 5B. U1 snRNA stem-loops 1 and 2 (55-MER). FIG. 5C. U1 small nuclear ribonucleoprotein A and 70 kDa.

FIG. 6 illustrates U1-70K in complex with U1 snRNA stem-loops and U1-A RRM in complex with stem-loop 2, stabilized through the U1-C zinc finger.

FIG. 7 illustrates a diagrammatic representation of the modulation of spliceosome machinery by an engineered polynucleotide (ASMO1, also known as APT20TTMG) described herein. The anchoring of the targeting moieties (“Stem 5′/3′”) (5′-GTCCA-3′ and 5′-CG-3′) allows interaction with the conserved site of constitutive donor, through the silencing of the RNA binding moiety (RBD) of U1 snRNA. Stabilization of the U1 snRNP complex can be observed through the strong ionic attraction of the Zinc Finger of U1-C, induced by disulfide bridges with thiol of the ASMO1 Stem 5′/3′. The pre-mRNA/ASMO1 duplex bond is stabilized by hydrogen bonds and electrostatic interactions between U1-C and the backbone of the pre-mRNA around the seam joint, but U1-C does not make base-specific contacts with pre-mRNA. The structure demonstrates that the selection of nucleotides of 5′-splices by U1 snRNP is achieved predominantly through the interaction between Stem 5′/3′ with pre-mRNA. Meanwhile, U1-C adjusts relative affinities of incompatible sites of 5′-splices and stabilizes the central core of spliceosome machinery by the interaction bridge between U1-70 kDa and the Sm ring. An electrostatic interaction and hydrogen bridges of Stem-Loop II with specific base (5′-AGGCC-3′) of an upper stem (a.k.a. “Hairpin-2”; see FIG. 2B) (3′-GGA-5′/5′-CCT-3′) and Internal loop: (3′-mCC-5′/5′-AA-3′); can be observed, associated with modulation of the polyadenylation signal and acetylation by U1-A. It is noted that the anchoring moiety of U1-A in Stem-Loop II (5′-CAACGUUA-3′) is not silenced by the upper stem, inducing the modulation of the levels of genic expression and acetylation. In addition, the presence of 2′-OME groups induces a change in the molecular dynamics of the medium facilitating the conformational alteration of U1-snRNA and approximation of Stem-loop II to ASMO1, where the ASMO1 targeting or recruiting moiety are distinct from the U1-A protein decreasing the likelihood of premature interruption of the reading frame by deregulation of the polyadenylation signal.

FIG. 8 illustrates U1-70K in complex with U1 snRNA stem-loops and U1-A RRM in complex with stem-loop 2, stabilized through the U1-C zinc finger. An electrostatic interaction and hydrogen bridges of Stem-Loop II with specific base (3′-CCGGA-5′) of an upper stem (3′-GGA-5′/5′-CCT-3′) and Internal loop: (3′-mCC-5′/5′-AA-3′); can be observed, associated with modulation of the polyadenylation signal and acetylation by U1-A. It is noted that the anchoring moiety of U1-A in Stem-Loop II (5′-CAACGUUA-3′) is not silenced by the upper stem, inducing the modulation of the levels of genic expression and acetylation. In addition, the presence of 2′-OME groups induces a change in the molecular dynamics of the medium facilitating the conformational alteration of U1-snRNA and approximation of Stem-loop II to the engineered polynucleotide described herein (ASMO1).

FIG. 9A illustrates U1-C sitting on SmD3 and its binding can be stabilized by the N-terminus of U1-70K. FIG. 9B illustrates U1-C forming hydrogen bonds with the sugar-phosphate backbone atoms but making no contact with RNA bases. On the 5′SS strand, nucleotides are colored teal for exonic and fawn for intronic sequence. FIG. 9C illustrates schematic representation of the 5′-splice site recognition. Red dotted lines: hydrogen bonds made by amino acid side chains of U1-C Zinc Finger. Blue dotted lines: hydrogen bonds made by main chain atoms of U1-C Zinc Finger. Green dotted lines: disulfide bonds made by amino acid side chains of the U1-C zinc finger. Orange dotted lines: disulfide bonds made by atoms in the main chain of the U1-C zinc finger. The 5′SS nucleotides are encoded by nuclei as in FIG. 9B.

FIG. 10A illustrates Fingerprint Z1 U1-C snRNP, represented by 36 amino acid residues in blue color. FIG. 10B illustrates Z1 finger moiety of U1-C snRNP, presenting the main residues that interact with the pre-mRNA/ASMO1 duplex in the 5′ constitutive donor region. FIG. 10C illustrates representative sequence of U1-C snRNP containing 145-aa, with the 36-aa highlighted in green refer to the Zinc Finger moiety.

FIG. 11A-D illustrates cell viability of U-87 cell line at 4 time points after incubation in ASMO1 (also known as “APT20TTMG”). FIG. 11A illustrates results from 24 hours of incubation. FIG. 11B illustrates results from 48 hours of incubation. FIG. 11C illustrates results from 72 hours of incubation. FIG. 11D illustrates results from 96 hours of incubation.

FIG. 12A-12D illustrates cell viability of MCF-7 cell line at 4 time points after incubation in ASMO1. FIG. 12A illustrates results from 24 hours of incubation. FIG. 12B illustrates results from 48 hours of incubation. FIG. 12C illustrates results from 72 hours of incubation. FIG. 12D illustrates results from 96 hours of incubation.

FIG. 13A-13D illustrates cell viability of SHSY5Y cell line at 4 time points after incubation in ASMO1. FIG. 13A illustrates results from 24 hours of incubation. FIG. 13B illustrates results from 48 hours of incubation. FIG. 13C illustrates results from 72 hours of incubation. FIG. 13D illustrates results from 96 hours of incubation.

FIG. 14A-C illustrates cell viability of PC-3 cell line at 3 time points after incubation in ASMO1. FIG. 14A illustrates results from 24 hours of incubation. FIG. 14B illustrates results from 48 hours of incubation. FIG. 14C illustrates results from 72 hours of incubation.

FIG. 15A-15C illustrates cell viability of 786-O cell line at 3 time points after incubation in ASMO1. FIG. 15A illustrates results from 24 hours of incubation. FIG. 15B illustrates results from 48 hours of incubation. FIG. 15C illustrates results from 72 hours of incubation.

FIG. 16 illustrates the kinetics of internalization of the compound ASMO1 by the breast carcinoma cell line MCF-7.

FIG. 17 illustrates the evaluation of mitochondrial activity in MCF-7 cell line after incubation with ASMO1.

FIG. 18 illustrates the distribution of cell cycle phases of the MCF-7 cell line after incubation with ASMO1.

FIG. 19 illustrates the distribution of MCF-7 phases after incubation with ASMO1.

FIG. 20 illustrates mitochondrial membrane potential in MCF-7 cell line after incubation with ASMO1.

FIG. 21 illustrates the kinetics of internalization of the compound ASMO1 by neuroblastoma cell line SH-SY5Y.

FIG. 22A-22B illustrates the evaluation of mitochondrial activity in SH-SY5Y cell line after incubation in ASMO1. MTT assay was performed after 24 hours (FIG. 22A) and 48 hours (FIG. 22B) of incubation.

FIG. 23 illustrates the distribution of cell cycle phases of the SH-SY5Y cell line after incubation with ASMO1.

FIG. 24 illustrates the distribution of SH-SY5Y phases after incubation with ASMO1.

FIG. 25 illustrates mitochondrial membrane potential in SH-SY5Y cell line after incubation with ASMO1.

FIG. 26 illustrates cell viability results obtained after 24, 48, 96 and 144 hours of incubation with ASMO1 (0.5 μM) in neuroblastoma cell line (SK-N-SH).

FIG. 27A-27B illustrates MAPT expression and tau quantification obtained after 24, 48, 96 and 144 hours of incubation with ASMO1 (0.5 μM) in neuroblastoma cell line. FIG. 27A shows MAPT expression after ASMO1 (0.5 μM) in different time points. FIG. 27B shows tau quantification after ASMO1 (0.5 μM) in different time points.

FIG. 28 illustrates the kinetics of internalization of the compound ASMO1 by glioblastoma cell line U87-MG.

FIG. 29 illustrates the distribution of cell cycle phases of the U87-MG cell line after incubation with ASMO1.

FIG. 30 illustrates the distribution of U87-MG phases after incubation with ASMO1.

FIG. 31 illustrates quantification of mitochondrial membrane potential in U87-MG cell line after incubation with ASMO1.

FIG. 32 shows the evaluation of mitochondrial activity in the U87-MG cell line after incubation with APT20TTMG.

FIG. 33 illustrates the evaluation of proliferation in U87-MG cell line with APT20TTMG.

FIG. 34A-34C illustrates the effect of intravenous administration of APT20TTMG in a glioblastoma xenograft model in athymic nude mice.

FIG. 35A-35B shows the effect of intravenous administration of APT20TTMG on the proliferation marker (Ki-67) in a glioblastoma xenograft model in athymic nude mice.

FIG. 36 shows the effect of intravenous administration of APT20TTMG on histopathological findings and tumor volume total score.

FIG. 37A-37D shows the effect of administration of APT20TTMG on the signaling pathway proteins in a glioblastoma xenograft model in athymic nude mice.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

Described herein are (e.g., engineered) polynucleotides, and (e.g., pharmaceutical) compositions and methods for utilizing the same, e.g., for modulating gene expression or activity.

Described herein are methods of treating cancer types using in the engineered polynucleotides described in this disclosure. The methods are applicable to various cancer types, such as cancers of different organs or cell types, or cancers associated or causes by different genetic aberrations. The engineered polynucleotides described herein may be effective at modulating splicing of pre-mRNA by the recruitment of components of the spliceosome. Without being bound by a particular mechanism, the improved recruitment of components of the spliceosome to a pre-mRNA may be effective at modulating the cellular mechanisms that are causative of cancer. The engineered polynucleotides may be administered to a subject that is suffering from cancer, and may treat the cancer, for example by decreasing the number of cancerous cells or preventing the proliferation or metastasis of the cancer cells.

Under physiological conditions, splicing is responsible for processing pre-messenger RNA (pre-mRNA), removing intronic regions, and joining exonic regions, thus producing a mature mRNA. Splicing is performed by a series of complexes of snRNPs and small nuclear RNAs (snRNAs). The U1 snRNPs complex is one of the five complexes that make up the human spliceosome (named U1, U2, U4, U5, and U6). The U1 snRNPs complex is composed of a U1 snRNA, seven Sm proteins forming a heptameric ring, and three other proteins: U1-70K, U1A, and U1C. In the spliceosome, the U1 snRNPs complex plays a specific role in the recognition of pre-mRNA splicing sites (splicing donor sites) in the early stages of spliceosome assembly. Then, other snRNPs, such as the U2 snRNPs, are attracted to the splicing site and interactions between their protein components complete the full spliceosome assembly and splicing. After that, the mature mRNA formed after undergoing chemical modifications of capping and polyadenylation may leave the cell nucleus and be translated into protein. In addition to the role in splicing, U1 snRNPs play a role in actively suppressing the polyadenylation machinery from using early, mostly intronic, polyadenylation signals that would lead to aberrant and truncated mRNAs. Accurate splicing is an essential step in gene expression, also enabling the alternative splicing that permits the balance between accuracy and flexibility of splice site recognition and creates from a single transcript multiple isoforms with diverse, sometimes even antagonistic, biological functions.

Beyond its splicing function, U1 snRNPs complex also has a telescripting function and actively inhibits the utilization of proximal polyadenylation signals (PAS), which are predominantly found within introns, by the polyadenylation machinery, suppressing a process known as premature polyadenylation. It is important to note that polyadenylation is one of the main processes of mRNA maturation and involves the termination of a pre-mRNA by the removal of a 3′ end portion, commonly in the 3′ untranslated (UTR) region, and the addition of a poly(A) tail. This process, along with the splicing and the 5′ capping, generates a mature mRNA, impacting transcript stability, export, and translation. The premature polyadenylation suppression by the U1 snRNPs complex prevents the production of aberrant and shortened mRNAs.

Additionally, U1 snRNPs have been described to be related to stress granules—cytoplasmic RNA granules, which contain mRNAs, associated translation initiation factors, and various RNA-binding proteins (such as snRNPs) that are formed in response to various stresses—and with autophagy, in lysosomal and autophagosome-lysosome biogenesis.

A cell cycle reentry triggered by U1 dysfunction in cells has been hypothesized as one of the factors that could lead to excessive proliferation and cancer. In the case of several types of cancer, the fundamental role of U1 snRNPs and snRNA in the suppression of premature cleavage and polyadenylation in RNA transcripts and the possible role of these snRNPs in cell migration and proliferation is observed.

In fact, many studies have shown the importance of alternative polyadenylation (APA) change and dysregulation in cancer and its progression. Alongside the splicing process, polyadenylation is a source of transcript diversity, as pre-mRNAs could have multiple alternative polyadenylation signals (PAS). The potential impact of APA on gene expression regulation and function has been seen in several studies. For example, well-differentiated cells like neurons commonly use distal PAS, located further downstream from stop codons, resulting in the expression of transcripts with longer 3′UTR and probably lower protein expression levels. Conversely, faster-growing cells, mainly cancer cells, tend to use proximal PAS, creating shorter 3′ UTR and potentially higher protein expression levels. The increased usage of proximal 3′ UTR PAS in multiple cancer cell lines and tissues has been associated with the promotion of cancer cell proliferation.

In addition to premature polyadenylation, there is evidence that spliceosomes are also altered in cancers like glioblastoma (GBM), resulting in the activation of oncogenic splicing events associated with tumor progression and severity. Among a well-defined group of adult-type diffuse gliomas, predominantly GBMs, it was observed a dysfunction in the expression of spliceosome constituents, such as the U1 small nuclear RNA (RNU1) and splicing factors that collaboratively recognize target introns. Notably, reductions in cell proliferation and migration, tumorsphere formation, and induction of apoptosis were observed upon silencing of specific splicing factors, such as RBM22, RBM3, PTBP1, and, especially, SRSF3. These effects are probably mediated through the modulation of key signaling pathways, such as PDGFRB and PI3K-AKT/ERK that are pro-oncogenic pathways in gliomas.

A variety of studies have shown the importance of these mechanisms in cancer, which is implicated in snRNP assembling, namely in U1snRNP biogenesis. For example, the potential relationship between these snRNPs and cell migration and proliferation has been identified in the human cervix epithelioid carcinoma (HeLa) cell line. This study shows that inhibition of U1 snRNA using the anti-sense morpholino oligonucleotide (U1 AMO), designed to target U1 snRNA, leads to widespread premature transcription termination and shortening of mRNA 3′-UTRs due to the usage of more proximal PAS in introns and last exons, resulting in the production of short mRNA isoforms. Interestingly, even lower concentrations of U1 AMO, which affect around 15-30% of U1 snRNA expression, increased migratory and invasive properties of cancer cells. Additionally, this phenomenon correlates with the upregulation of oncogenes and the downregulation of tumor suppressor genes. The opposite effect is observed when U1 snRNA is overexpressed, reducing migration and invasion of cancer cells. In a contrasting study, also utilizing HeLa cells, overexpression of U1 snRNA reversed DNA damage, particularly induced by ultraviolet treatment. This DNA damage was characterized primarily by decreased U1 snRNA levels alongside substantial regulation of intronic alternative cleavage and polyadenylation in the 3′ UTR, leading to both 3′ UTR shortening and lengthening of genes and expression of truncated transcripts. Overall, these studies emphasize the critical role of polyadenylation in modulating the fate of cancer cells.

Another study in hepatocellular carcinoma (HCC), U1A expression (confirmed by mRNA expression and immunohistochemistry validation) was positively correlated with tumor stage and grade, being an independent poor prognostic factor for HCC. A knockdown of U1A, to further access U1A role in HCC, inhibited migration and cell cycle progression while promoting the apoptosis of HCC cell lines. Moreover, an association between U1A mRNA expression and tumor-infiltrating immune cells was also observed. Another study involving HCC cell lines and HeLa shows that a knockdown in U1A also causes a strong drop in CCN2 expression and connective tissue growth factor (CTGF) secretion (CTGF protein is encoded by CCN2 oncogene), decreasing cell migration and proliferation. It is noteworthy that CTGF is involved in cell proliferation, angiogenesis, and migration, a phenomenon important to epithelial-mesenchymal transition and, ultimately, metastasis. Likewise, U1A is upregulated in lung adenocarcinoma (LUAD) and lung squamous cell carcinoma tissue (LUSC). The high U1A expression was associated with a poor first- and post-progression survival prognosis of LUAD cases. However, a rate of 4% of genetic alteration (like missense mutation, and deep deletion) of U1A was associated with the overall survival prognosis of LUSC cases, but not LUAD cases. In both LUAD and LUSC, U1A expression was negatively correlated with the infiltration level of M2 macrophage but positively correlated with that of Follicular B helper T cells. Moreover, in the LUAD pathogenesis, U1A expression was correlated with genes involved in the cell cycle and ubiquitin mechanism-associated issues, while a correlation with RNA splicing-related cellular issues (e.g., spliceosome, RNA splicing, pre-mRNA binding, alternative mRNA splicing, etc.) were mainly observed for LUSC.

Taking together, these U1 snRNPs studies with different cancer types could suggest that U1 homeostasis is important to maintain the expression balance of normal gene isoforms and that U1 snRNPs disbalance could impact the proliferation, migration, and invasion of cancer cells. Consequently, the modulation of U1 could be a target for oncology therapy. In fact, modulating the activity of U1 snRNP in cancers has the potential to correct the pathological effects resulting from splicing and premature polyadenylation dysfunctions, as, under physiological conditions, U1 snRNP is involved in both mechanisms. U1 snRNP is an essential component of the splicing machinery responsible for selecting splicing sites and facilitating spliceosome assembly by binding to the 5′ splicing site, known as the donor splicing site, that typically contains GU sequences. It also plays a crucial role in suppressing premature cleavage and polyadenylation at the 3′ end of pre-mRNAs, specifically within the GU-rich regions located within introns. This suppression mechanism prevents the recognition of these cryptic regions by the polyadenylation machinery, which would otherwise lead to the generation of truncated and abnormal mRNA transcripts. Additionally, it is important to highlight the role of U1snRNP subunits, such as U1-A, U1-C, and U1-70K proteins, which have been identified as key regulators of both splicing and polyadenylation. As a result, a correct function of U1 snRNP and its telescripting function, encompassing the suppression of premature polyadenylation and cryptic splicing, safeguards pre-mRNAs and contributes to the regulation of alternative polyadenylation, thereby avoiding pro-oncogenic process.

In addition to the effect on U1 snRNPs complex modulation, other evidence that reveals the potential of ASMO for applications in the treatment of cancer is its capacity to regulate tau expression: a neuroblastoma SK-N-SH cell lineage treated with different concentrations of APT20TTMG (ASMO1) showed a reduction of tau expression at the mRNA and protein level along with cell viability results that indicate a cytostatic potential effect upon APT20TTMG treatment. In addition, the in vivo study with human glioblastoma cancer (U-87MG) xenograft mice model also showed a reduction in Tumor tau expression in relation to the control group along with tumor volume reduction upon APT20TTMG treatment. These results are interesting since there is emerging evidence of the tau protein's role in cancer. Furthermore, as a microtubule-binding protein, tau may interfere with the binding of taxanes (microtubule-stabilizing drugs) to tubulin, hence, anti-tau molecules could be a strategy to improve the effect of taxane-based chemotherapies.

In breast cancer cell lines, for example, a relationship between tau expression and response to taxanes was demonstrated: tau knockdown, by RNA interference, increased the sensitivity to taxanes (paclitaxel and docetaxel) in both ZR75-1 and MCF-7 cell lines, what is suggested to be associated with tau protein isoforms of less than 70 kDa. Also, estrogen receptor (ER) signaling was demonstrated to affect the expression of tau protein isoforms of less than 70 kDa, which can impact sensitivity to taxanes. Furthermore, Fulvestrant, a selective estrogen receptor degrader used as a medication to treat metastatic breast cancer, decreased ER and tau expression and its combination with taxanes increased the sensitivity of tau- and ER-positive breast cancer cells to taxanes. Despite this, a study showed that among the ER-positive patients, patients with tau-positive cancers had better disease-free survival and overall survival compared to tau-negative tumors, but there was no significant interaction between tau expression and benefit from paclitaxel. Among the ER-negative patients of the study, tau expression had no prognostic value. A question that was raised by the authors is why a large, randomized study did not corroborate the preclinical findings, as previously described. A suggested answer is that the role of tau in resistance to paclitaxel (among other resistance causes) is possibly clinically relevant in only a small set of patients. But this discrepancy could be caused also by chemotherapy regimen differences, for example. Contrary to what was observed in patients with breast cancer, high tau expression is associated with poor prognosis in prostate cancer (PC). Moreover, the knockdown of tau suppressed the expression of the androgen receptor (AR plays a decisive role in the progression to castration-resistant PC) and increased the sensitivity to bicalutamide, an anti-androgen drug that stops testosterone from reaching the cancer cells. A study with prostate tumor cell line, ALVA-31 derivatives (ALVA-NEO and ALVA-hCD40), showed that, besides other isoforms, all six alternatively spliced adult brain tau isoforms are expressed and are highly phosphorylated, with a substantial proportion not binding to microtubules. Besides, ALVA-NEO tau interacts with PI3K/AKT, which is associated with cell survival and proliferation in many cancers. Consistent with this, the downregulation of tau, in taxane-resistant prostate cell lines, inhibits cell proliferation by the PI3K/Akt/mTOR signaling pathway, sensitizing to taxane cytotoxicity.

In GBM, for example, tau downregulation by a short hairpin interference RNA (shRNA) approach in the U87-MG cell line significantly reduces 2D-cell motility by inducing an inefficient cell-tail retraction through relocation of ROCK. Rho-ROCK signaling is known to act on cell migratory and invasive phenotypes by regulating the actin cytoskeleton. In this study, it was observed that tau helps with the remodeling of the microtubules and actin cytoskeletons, both of which are essential for migration. This trait could be produced by the restricted activity of the Rho-ROCK pathway, which is involved in the regulation of polymerization in the back of cells. All these effects are initiated upstream by tau-induced microtubule bundling and stabilization. The depletion of tau disturbs this microtubule and actin assembly balance at the back of U87MG cells, also disturbing the cell retraction of the back important in cell migration. It is worth mentioning that among the GBM cell lines (U118, U138, and U251), U87-MG cells expressed the highest amount of tau. Another more recent study, using shRNA for the knockdown of tau in a 3D model of multicellular spheroids (MCS) of U87-MG cells, showed inhibition of MCS growth and cell evasion by impacting cell migration and spheroid cohesion. It was also observed a decrease in MCS compactness, due to an N-cadherin mislocalization. Moreover, in a glioblastoma xenograft model, mice injected with U87MG sh-tau cells (cells with reduced levels of tau) showed a significantly higher median survival than mice injected with U87MG control cells. Because of these results, the authors suggested a role for tau in glioblastoma by controlling 3D cell organization and functions via the PI3K/AKT signaling axis.

Also, in this MCS study, a model was proposed for the role of tau in controlling PI3K/AKT signaling and N-cadherin-β-catenin. N-cadherin junctions are responsible for cell-cell adhesion necessary for spheroid compactness and the cadherin-catenin complex at the membrane was already demonstrated to recruit PI3K, initiating the signaling cascade. tau may reinforce these interactions through the stabilization of the microtubule networks. Specifically, in PTEN-null mutated GBM cells (PTEN loss is very common in GBM, which could stimulate an invasive behavior), tau is proposed to promote stabilization and compactness of N-cadherin/β-catenin complexes through microtubule-dependent interactions, reinforcing cell-cell adhesion. tau is also suggested to be involved in cell contraction through the promotion of actin assembly. An indirect activation cascade of the PI3K-AKT signaling pathway is also stimulated by tau through MT-independent interactions or by increased N-cadherin-β-catenin signaling, resulting in cell survival and proliferation. In tau-depleted U87MG cells, a defective recruitment of N-cadherin-β-catenin complexes to the actin cytoskeleton is proposed. In this case, N-cadherin is not stabilized at the membrane, resulting in a loss of MCS compactness. Besides, a loss of cell-cell cohesion is proposed to be related to the sequestration of β-catenin by mislocalized N-cadherin and due to a decrease in PI3K-AKT signaling activity. A reduction of phosphorylated Akt kinase is also observed. As a consequence of this suggested mechanism, inhibition of cell proliferation and migration are expected in tau-depleted GBM cells. Besides, it is interesting to note that tau protein is described to have a wide interactome, including with cancer-related kinase proteins, which could also have an impact on cellular pathways such as cell signaling, cell motility, and cellular metabolism, involving other cascade mechanisms.

Differently from glioblastoma studies, a study with cell renal cell carcinoma line shows that downregulation of tau enhanced cell growth and invasion in 786-O cell lines and tau may play a tumor-suppressive role in that type of cancer. Similarly, in neuroblastoma cell SH-SY5Y lines, tau knockout by CRISPR-Cas9 technology and knock-down by RNA interference (shRNA) caused altered dysregulation of P53, a pro-apoptotic tumor suppressor, and activity resulting in reduced DNA damage-induced apoptosis and increased cell senescence. In accordance with this tau beneficial effect in neuroblastoma, another study shows that patients with higher tau mRNA expression (measured by microarray and with RNA-seq data) were associated with a significantly increased overall survival rate in pediatric neuroblastoma. The result that higher tau expression correlates with better outcomes was consistent with higher expression of certain apoptosis-effector genes (mainly CASP3 and CASP9) among the samples represented by the high tau expression and lower expression of the pro-proliferative histone genes among the samples that had a high tau expression. Patients with lower tau expression, which also had decreased overall survival, also had a significantly greater incidence of MYCN amplification (classically associated with neuroblastoma worse outcomes). These cases where tau expression is associated with a good outcome could be related to a stabilization of microtubules resulting in a cytostatic effect, which is a mechanism similar to microtubule-stabilizing agents like paclitaxel that are widely used in cancer treatment. Accordingly, the present disclosure provides engineered polynucleotides that function as an oncology therapy, and corresponding methods of using the engineered polynucleotides. The engineered polynucleotides may function through a mechanism of homeostatic modulation of U1 snRNPs function, regulation of tau expression, a combination (e.g., synergistically) of both, and cause a cytostatic or cytotoxic effect on the chosen types of cancers, allowing for a broad application on oncology therapy. To allow this U1 modulation, the engineered polynucleotides can be designed to have size, conformation, and strategic chemical modifications that allow the attraction and interaction with U1C directly, and U1-70K indirectly. The engineered polynucleotides (e.g., ASMO1) can have a sequence complementary to highly conserved regions present pre-mRNAs in general at exon-intron junctions at the 5′ end of introns—called donor splicing sites. The U1 modulation can ensure the correct assembly (with the right distance, position, and behavior) of the snRNPs that make up the U1 complex, which is highly relevant in the splicing process. As splicing could control patterns of expression at a post-transcriptional level, in cells with abnormal gene expression caused by U1 dysfunction, a U1 modulation triggered by the engineered polynucleotides (e.g., ASMO1) could help protein expression normalization. The regulation of tau expression upon treatment with the engineered polynucleotides (e.g., ASMO1) could be a response to this splicing modulation in dysregulated cells, what could be useful for cancer therapy. Altogether, the key roles of U1 snRNPs and tau protein in cancer migration and progression, and our in vitro and in vivo findings indicate that the engineered polynucleotides contemplated in this disclosure can act as a therapeutic agent for the treatment of cancers like GBM.

### Engineered Polynucleotide(s)

The present disclosure provides engineered polynucleotides and methods of using the engineered polynucleotides. Engineered polynucleotides generally refer to polynucleotides that are not naturally occurring. These engineered polynucleotides are not limited to any form of synthesis and can be generated via any method of synthesis (e.g, recombinant technologies, or solid phase synthesis). An engineered polynucleotide or an engineered polynucleotide as described herein can include various moieties. A “moiety” can refer to a region of an engineered polynucleotide. In some cases, a moiety can be described in terms of a function of the moiety. For instance, a “targeting moiety” can refer to a region of the engineered polynucleotide that can be at least partially complementary to a target RNA; a “recruiting moiety” can refer to a moiety that can recruit any one of the regulating moiety described herein; and a “spacing sequence” can refer to a moiety that provides space between other moieties. In some instances, recitation of a moiety name does not limit the moiety to a particular function. For example, a “targeting moiety” that can be at least partially complementary to a target RNA can in some instances recruit a regulating moiety. The various moieties as described throughout this disclosure can be combined to generate engineered polynucleotides, which can perform a particular function. For example, an engineered polynucleotides can comprise a targeting moiety disclosed herein with a recruiting moiety disclosed herein.

In some embodiments described herein, the engineered polynucleotide comprises: (i) one or more targeting moiety configured to (e.g., specifically) bind a ribonucleic acid (RNA) (e.g., a messenger ribonucleic acid (mRNA), such as a pre-messenger ribonucleic acid (pre-mRNA)) at a target sequence therein. The engineered polynucleotide may further comprise (ii) a recruiting moiety configured to recruit a post-transcriptional regulating moiety (e.g., a spliceosomal moiety) such that, when associated with the RNA (e.g., the mRNA, such as the pre-mRNA) and the engineered polynucleotide, the post-transcriptional regulating moiety alters the RNA (e.g., the mRNA, such as the pre-mRNA) in or in proximity to the target sequence. In some embodiment, “configured to specifically bind to” refers to hybridizes to. In some embodiments, a moiety configured to specifically bind to a pre-mRNA refers to a moiety that is at least 80%, 90%, or 100% complementary to the pre-mRNA sequence to which it specifically binds. In some embodiments, the RNA (e.g., the mRNA, such as the pre-mRNA) encodes a target gene. In some embodiments, a pre-mRNA associated with an engineered polynucleotide refers to a pre-mRNA hybridized with at least one nucleotide (and up to about 100%) of the engineered polynucleotide. In some embodiments, “alters the RNA” refers to modifying a nucleic acid molecule by cleaving a nucleic acid, reacting a nucleotide of the nucleic acid, or splicing the nucleic acid. In some embodiments, “proximity” refers to a distance of no more than 5 nucleotides away.

### Targeting Moieties

In various aspects, the engineered polynucleotides comprise one or more targeting moiety. The targeting moiety may allow the engineered polynucleotide to interact with ribonucleic acid (e.g., a pre-mRNA). The targeting moiety may comprise sequences that are complementary or identical to sequence of the ribonucleic acid (e.g., a pre-mRNA) such that the engineered polynucleotide and ribonucleic acid can interact or hybridize. The targeting moiety may be complementary or identical to a target sequence. In some embodiments of the engineered polynucleotide described herein, a targeting moiety of the one or more targeting moiety is sufficiently identical or complementary to a consensus sequence in the target sequence. The target sequence may be in a target gene. The target sequence may be a conserved or consensus sequence that is present in multiple different pre-mRNA. For example, the target sequence may allow the engineered polynucleotides to interact with a first pre-mRNA that encodes for one gene and a second pre-mRNA for another gene. Targeting moieties can be found at the 5′ end and 3′ end of an engineered polynucleotide and may also be referred to as lower stems or legs. Without wishing to be bound by theory, stable binding of one or two targeting moieties to a constitutive donor 5′ of a target pre-mRNA may allow the interaction with the conserved site of the constitutive donor and may silence the U1 snRNA RNA-binding domain (RBD). The targeting moieties may comprise sequences that are substantially identical to a sequence of the U1 snRNA RBD. For example, the RBD may comprises the following sequence: 3′-GUCCAUUCAUA-5′ (SEQ ID NO: 6), and the targeting moiety may comprise GTCCA (or GUCCA). The targeting moieties may effectively displace the U1 snRNA RBD from bind the pre-mRNA or prevent binding of the U1 snRNA RBD to the pre-mRNA, based on the on the sequence similarity of the U1 snRNA RBD and the targeting moiety.

A consensus sequence can be determined based on identification of genetic variants of unknown significance (VUS). Any exonic or intronic VUS can be spliceogenic by disrupting the cis DNA sequences that define exons, introns, and regulatory sequences necessary for a correct RNA splicing process. The cis DNA elements can include: exon-intron boundary core consensus nucleotides (e.g., GT at +1 and +2 of the 5′donor site and AG at −1 and −2 of the 3′acceptor site); or intronic and exonic nucleotides adjacent to these invariable nucleotides that are also highly conserved and have been found to be involved for splice site selection (e.g., CAG/GUAAGU in donor sites and NYAG/G in acceptor sites). A nucleotide change in any of these elements can lead to incorrect splice site recognition, creating a new splice site or activating a cryptic splice site, resulting in aberrant transcripts or non-functional proteins associated with a disease or condition. In some embodiments, at least one targeting moiety of the one or more targeting moiety is sufficiently identical or complementary to a consensus sequence in the target sequence of a target ribonucleic acid or target gene. In some embodiments, the consensus sequence comprises about 2 to about 15 nucleotides, about 2 nucleotides to about 10 nucleotides, or about 2 nucleotides to about 8 nucleotides. In some embodiments, at least two targeting moieties of the one or more targeting moiety are sufficiently identical or complementary to at least two consensus sequences in the target sequence of a target gene or target ribonucleic acid. In some embodiments, the one or more targeting moiety are each independently sufficiently identical or complementary to a consensus sequence in the target sequence of a target gene or target ribonucleic acid.

In some embodiments of the engineered polynucleotide described herein, the one or more targeting moiety each independently comprises about 2 to about 15 nucleotides, about 2 nucleotides to about 10 nucleotides, or about 2 nucleotides to about 8 nucleotides. In some embodiments of the engineered polynucleotide described herein, the one or more targeting moiety each independently comprises 2, 3, 4, 5, 6, 7, 8, 9, or 10 nucleotides

In some embodiments of the engineered polynucleotide described herein, the one or more targeting moiety comprises (1) a first targeting moiety configured to specifically bind a first targeted sequence in the target sequence of the RNA (e.g., the mRNA, such as the pre-mRNA), and (2) a second targeting moiety configured to specifically bind a second targeted sequence in the target sequence of the RNA (e.g., the mRNA, such as the pre-mRNA). In some embodiments, the first targeted sequence comprises a consensus sequence in the target sequence. In some embodiments, the consensus sequence of the first targeted sequence comprises about 2 to about 15 nucleotides, about 2 nucleotides to about 10 nucleotides, or about 2 nucleotides to about 8 nucleotides. In some embodiments, the consensus sequence of the first targeted sequence comprises 2-3, 3-4, 4-5, 5-6, 6-7, 7-8, or 8-10 nucleotides. In some embodiments, the second targeted sequence comprises a consensus sequence in the target sequence. In some embodiments, the consensus sequence of the second targeted sequence comprises about 2 to about 15 nucleotides, about 2 nucleotides to about 10 nucleotides, or about 2 nucleotides to about 8 nucleotides. In some embodiments, the consensus sequence of the second targeted sequence comprises 2-3, 3-4, 4-5, 5-6, 6-7, 7-8, or 8-10 nucleotides. In some embodiments, the consensus sequence of the first targeted sequence and the consensus sequence of the second targeted sequence are of different nucleotide lengths. In some embodiments, one of the consensus sequences of the first and second targeted sequences comprises about 1 to about 5 nucleotides, and the other of the consensus sequences of the first and second targeted sequences comprises about 4 to about 8 nucleotides. In some embodiments, one of the consensus sequences of the first and second targeted sequences comprises at least about 2 nucleotides, and the other of the consensus sequences of the first and second targeted sequences comprises at least about 5 or 6 nucleotides. In some embodiments, one of the consensus sequences of the first and second targeted sequences comprises about 2 nucleotides, and the other of the consensus sequences of the first and second targeted sequences comprises about 5 or 6 nucleotides.

In some embodiments of the engineered polynucleotide described herein, a first targeting moiety and a second targeting moiety are of different nucleotide lengths. In some embodiments, one of a first targeting moiety and a second targeting moiety comprises about 1 to about 5 nucleotides, and the other of a first targeting moiety and a second targeting moiety comprises about 4 to about 8 nucleotides. In some embodiments, one of a first targeting moiety and a second targeting moiety comprises at least about 2 nucleotides, and the other of a first targeting moiety and a second targeting moiety comprises at least about 5 or 6 nucleotides. In some embodiments, one of a first targeting moiety and a second targeting moiety comprises about 2 nucleotides, and the other of a first targeting moiety and a second targeting moiety comprises about 5 or 6 nucleotides.

In some embodiments, a (e.g., first or second) targeting moiety comprises a nucleic acid sequence having at least one, two, three, four, five, six, seven, eight, nine, ten, or more nucleotides. In some embodiments, a (e.g., first or second) targeting moiety comprises a nucleic acid sequence having at most ten, nine, eight, seven, six, five, four, three, or two nucleotides. In some embodiments, a (e.g., first or second) targeting moiety comprises a nucleic acid sequence having one, two, three, four, five, six, seven, eight, nine, or ten nucleotides, or a range between any two foregoing values.

In some embodiments, the (e.g., first or second) targeting moiety comprises a nucleic acid sequence targeting the nucleotides in an exon. In some embodiments, the (e.g., first or second) targeting moiety comprises a nucleic acid sequence targeting the nucleotides in an exon that are immediately adjacent to an intron. In some embodiments, the (e.g., first or second) targeting moiety comprises a nucleic acid sequence targeting the nucleotides in an exon that are adjacent to or immediately adjacent to an intron that is at the 3′ end of the exon. In some embodiments, the (e.g., first or second) targeting moiety comprises a nucleic acid sequence targeting the nucleotides in an exon that are adjacent to or immediately adjacent to an intron that is at the 5′ end of the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon. In some embodiments, the (e.g., first or second) targeting moiety comprises a nucleic acid sequence targeting the nucleotides in an exon that is immediately adjacent to an intron that is at the 5′ end of the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is adjacent to the an intron. In some embodiments, the (e.g., first or second) targeting moiety comprises a nucleic acid sequence targeting the nucleotides in an exon that is immediate adjacent to an intron that is at the 5′ end of the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is at the 5′ of an intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is at the 3′ of an intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′—CC-3′, 5′-CG-3′, 5′-GA-3′, 5′-GT-3′, 5′-GC-3′, or 5′-GG-3′ targeting the nucleotides that are in the exon that is not immediately adjacent to an intron.

In some embodiments, the (e.g., first or second) targeting moiety comprises a nucleic acid sequence targeting the nucleotides in intron. In some embodiments, the (e.g., first or second) targeting moiety comprises a nucleic acid sequence targeting the nucleotides in an intron that are adjacent to or immediately adjacent to the exon. In some embodiments, the (e.g., first or second) targeting moiety comprises a nucleic acid sequence targeting the nucleotides in an intron that are adjacent to or immediately adjacent to the exon that is at the 5′ end of the intron. In some embodiments, the (e.g., first or second) targeting moiety comprises a nucleic acid sequence targeting the nucleotides in an intron that are adjacent to or immediately adjacent to the exon that is at the 5′ end of the intron. In some embodiments, the (e.g., first or second) targeting moiety comprises a nucleic acid sequence targeting the nucleotides that are located entirely within an intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in an intron that is adjacent to the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the intron and immediately adjacent to the exon that is at the 5′ end of the intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the intron and immediately adjacent to the exon that is at the 3′ end of the intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the intron that is not adjacent to an exon.

As described, a targeting moiety (e.g., first or second) may be gene agnostic such that it can target conserved regions that are shared (or are substantially similar) in pre-mRNA of multiple different genes. Additionally, targeting moiety (e.g., first or second) may target a specific gene, or specific exon and intron of a gene. As an example, Table 1 provides the MAPT gene Exon-Intron junction sequences for multiple exons-introns junctions. For example using Table 1 (or other sequences from a gene or exon-intron junctions of a gene of interest), engineered polynucleotides can be designed such to target a specific exon-intron junction, or may be designed to target more than one exon-intron junction. In some embodiments of the engineered polynucleotide described herein, a (e.g., first or second) targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in Table 1. In some embodiments of the engineered polynucleotide described herein, a (e.g., first or second) targeting moiety comprises a sequence identical or complementary to a sequence set forth in Table 1. In some embodiments, a (e.g., first or second) targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence selected from the “Exon sequence” column of Table 1 and the “Intron sequence” column of Table 1. In some embodiments, a (e.g., first or second) targeting moiety comprises a sequence identical or complementary to a sequence selected from the “Exon sequence” column of Table 1 and the “Intron sequence” column of Table 1.

In some embodiments, a first targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence selected from the “Exon sequence” column of Table 1; and a second targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in the “Intron sequence” column of Table 1. In some embodiments, a first targeting moiety comprises a sequence identical or complementary to a sequence selected from the “Exon sequence” of Table 1; and a second targeting moiety comprises a sequence identical or complementary to a sequence set forth in the “Intron sequence” column of Table 1. In some embodiments, a first targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in the “Intron sequence” column of Table 1; and a second targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in the “Exon sequence” of Table 1. In some embodiments, a first targeting moiety comprises a sequence identical or complementary to a sequence set forth in the “Intron sequence” column of Table 1; and a second targeting moiety comprises a sequence identical or complementary to a sequence set forth in the “Exon sequence” column of Table 1. In some embodiments, a (e.g., first or second) targeting moiety comprises a sequence identical or complementary to a consensus sequence of an intron donor site (e.g., selected from GU, GT, GC, and CA). In some embodiments, a (e.g., first or second) targeting moiety comprises a sequence identical or complementary to a consensus sequence of an exon donor site (e.g., G). In some embodiments, a (e.g., first or second) targeting moiety comprises a sequence identical or complementary to a consensus sequence selected from GU, GC, G, and CA.

TABLE 1
Examples of MAPT gene Exon-Intron junction
sequences targeted by the 5′ and 3′-targeting
moieties of engineered polynucleotides and
the distance between the two target
sequences

| | | | Intron |
|---|---|---|---|
| Exon | Exon sequence | Spacer | sequence |
| Exon 1 | 5′-CAGGT-3′ | 2 nt | 3′-CG-5′ |
| Exon 2 | 5′-AAGGT-3′ | 2 nt | 3′-TG-5′ |
| Exon 3 | 5′-AAGGT-3′ | 2 nt | 3′-CG-5′ |
| Exon 4 | 5′-CAGGT-3′ | 2 nt | 3′-GG-5′ |
| Exon 5 | 5′-AAGGT-3′ | 2 nt | 3′-TG-5′ |
| Exon 6a | 5′-AAGGT-3′ | 2 nt | 3′-TG-5′ |
| Exon 6b | 5′-AAGGT-3′ | 2 nt | 3′-TG-5′ |
| Exon 7 | 5′-AAGGT-3′ | 2 nt | 3′-CG-5′ |
| Exon 8 | 5′-AAGGT-3′ | 2 nt | 3′-GG-5′ |
| Exon 9 | 5′-CTGGT-3′ | 1 nt | 3′-AG-5′ |
| Exon 10 | 5′-CTGGT-3′ | 2 nt | 3′-AG-5′ |
| Exon 11 | 5′-AGTGT-3′ | 2 nt | 3′-TG-5′ |
| Exon 12 | 5′-CAGGT-3′ | 1 nt | 3′-CG-5′ |
| Exon 13 | 5′-AAGGT-3′ | 3 nt | 3′-GG-5′ |
| Exon 14 | 5′-AAGGT-3′ | 2 nt | 3′-CC-5′ |
| Exon 1 | 5′-AAAAG-3′ | 3 nt | 3′-CGA-5′ |
| Exon 2 | 5′-AAAAG-3′ | 3 nt | 3′-TGA-′5 |
| Exon 3 | 5′-GGAAG-3′ | 3 nt | 3′-GGG-′5 |
| Exon 4 | 5′-CACAG-3′ | 3 nt | 3′-GGA-5′ |
| Exon 5 | 5′-CCAAG-3′ | 3 nt | 3′-TGA-5′ |
| Exon 6a | 5′-CAAAG-3′ | 3 nt | 3′-TGT-5′ |
| Exon 6b | 5′-CAAAG-3′ | 3 nt | 3′-TGT-5′ |
| Exon 7 | 5′-CCAAG-3′ | 3 nt | 3′-CGT-5′ |
| Exon 8 | 5′-TCAAG-3′ | 3 nt | 3′-GGA-5′ |
| Exon 9 | 5′-CTCTG-3′ | 3 nt | 3′-AGA-5′ |
| Exon 10 | 5′-GGAAG-3′ | 3 nt | 3′-AGA-5′ |
| Exon 11 | 5′-GGCAG-3′ | 3 nt | 3′-AGA-5′ |
| Exon 12 | 5′-AACAG-3′ | 2 nt | 3′-CGA-5′ |
| Exon 13 | 5′-AAAAG-3′ | 3 nt | 3′-GAA-5′ |
| Exon 14 | 5′-TTAAA-3′ | 2 nt | 3′-GGA-5′ |

Example consensus sequences include: (e.g., 5′-) intron donor site #1: GU; (e.g., 5′-) intron donor site #2: GC; (e.g., 5′-) exon donor site #1: G; and (e.g., 5′-) intron donor site #3: CA.

A targeting moiety (e.g., first or second) may be specific to a gene, or specific exon and intron of a gene. For example, the targeting moiety may be specific to a exon-intron junction and a portion of the exon or intron. In some embodiments, the targeting moiety is specific to a portion of the gene that is outside of the splice site. In some embodiments, the targeting moiety targets a specific gene. For example, the targeting moiety may comprise a section that is specific to the splice site and a specific to the gene. As described elsewhere herein, the targeting moiety may comprise sequences of consensus splice site. By having a targeting moiety of specific to consensus splice sites, the targeting moiety may be gene agnostic and target multiple different mRNA of different genes. Alternatively, the targeting moieties may comprise a portion that is specific to a gene. The engineered polynucleotide may then be able to modulate the splicing of a specific target gene (as opposed to any gene/mRNA that comprises a consensus splice site). For example, the targeting moieties may comprise sequences complementary to sequences unique to MAPT.

### Target Sequence

As described in this disclosure, the engineered polynucleotides may comprise one or more targeting moieties. The targeting moieties may target (e.g., bind to, hybridize to, or otherwise be configured to interact with) a target sequence, for example, where the targeting moieties target adjacent or nearby regions in a target sequence. The targeting moieties may be complementary to a targeted sequence. In some embodiments of the engineered polynucleotide described herein, a first targeted sequence and a second targeted sequence are apart in the target sequence by a spacing sequence of no more than five, four, or three nucleotides (e.g., one or two nucleotides).

In some embodiments of the engineered polynucleotide described herein, a first targeted sequence and a second targeted sequence are contiguous or adjacent to each other.

In some embodiments, when a spacing sequence in the target sequence is adjacent to a 5′- or 3′-end of a targeted sequence of the target sequence, the spacing sequence may not be complementary to a targeting moiety of the engineered polynucleotide. In some embodiments, when a spacing sequence in the target sequence is adjacent to a 5′- or 3′-end of a targeted sequence of the target sequence, the spacing sequence may not be complementary to any targeting moiety of the engineered polynucleotide.

In some embodiments, the spacing sequence separate a first targeted sequence and a second targeted sequence described herein. In some embodiments, the spacing sequence is not complementary and does not bind to a targeting moiety of the engineered polynucleotide. In some embodiments, the spacing sequence is not complementary and does not bind to all targeting moieties of the engineered polynucleotide.

In some embodiments of the engineered polynucleotide described herein, the target sequence may comprise an exon-intron boundary in the RNA (e.g., the mRNA, such as the pre-mRNA). In some embodiments, a first targeted sequence and a second targeted sequence are both 5′ or 3′ with respect to the exon-intron boundary. In some embodiments, one of a first targeted sequence and a second targeted sequence is 5′ with respect to the exon-intron boundary, and the other of a first targeted sequence and a second targeted sequence is 3′ with respect to the exon-intron boundary.

In some embodiments of the engineered polynucleotide described herein, the target sequence comprises a splice site in the RNA (e.g., the mRNA, such as the pre-mRNA). In some embodiments, a (e.g., first or second) targeted sequence comprises a splice site (e.g., 5′ ss) in the RNA (e.g., the mRNA, such as the pre-mRNA).

In some embodiments, two targeted sequences (e.g., a first targeted sequences and a second targeted sequence) are part of a single nucleic acid molecule (i.e., the RNA, e.g., the mRNA, such as the pre-mRNA). In some embodiments, the first and second targeted sequences are spanned apart on the single nucleic acid molecule. In some embodiments, the first and second targeted sequences span an exon-intron boundary of the single nucleic acid molecule. In some embodiments, the first and second targeted sequences do not span an exon-intron boundary of the single nucleic acid molecule. In some embodiments, the first and second targeted sequences are adjacent to an exon-intron boundary of the single nucleic acid molecule. In some embodiments, the first and second targeted sequences both target an intron of the single nucleic acid molecule. In some embodiments, the first and second targeted sequences span a splice site in the single nucleic acid molecule. In some embodiments, the first and second targeted nucleic acid sequences do not span a splice site in the single nucleic acid molecule.

In some embodiments of the engineered polynucleotide described herein, a consensus sequence in the target sequence comprises about 2 to about 15 nucleotides, about 2 nucleotides to about 10 nucleotides, or about 2 nucleotides to about 8 nucleotides.

In some embodiments, the engineered polynucleated in complementary and binds to a target sequence. In some embodiments, at least a portion of the engineered polynucleotide binds to a target sequence. In some embodiments, the target sequence encodes a target gene. Non-limiting example of the target gene can include microtubule associated protein tau (MAPT).

In some embodiments, the target sequence comprises an RNA sequence. In some embodiments, the RNA is a nuclear RNA, a cytoplasmic RNA, or a mitochondrial RNA. In some embodiments, the target RNA sequence comprises a messenger RNA (mRNA), a pre-messenger RNA (pre-mRNA), a transfer RNA (tRNA), a ribosomal RNA (rRNA), a ribozyme, a recombinant polynucleotide, a branched polynucleotide, an isolated RNA, an guide RNA, an oligonucleotide, a nucleic acid probe, a primer, an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA). In some embodiments, the target RNA sequence is a pre-mRNA. In some embodiments, the engineered polynucleotide is not an antisense oligonucleotide.

In some embodiments, the target RNA sequence comprises at least one exon. In some embodiments, the target RNA sequence comprises at least one intron. In some embodiments, the target RNA sequence comprises at least one exon or at least one intron. In some embodiments, the target RNA sequence comprises at least one exon-intron boundary. The target RNA sequence may comprise a sequence specific to a gene. For example, the target RNA sequence may comprise sequences encoding for a polypeptide. For example, the target RNA sequence may comprise sequences encoding for a tau. The target sequence may comprise a sequence corresponding to an exon-intron boundary and a sequence corresponding to a specific gene.

In some embodiments, the target sequence is an endogenous nucleic acid molecule. In some embodiments, the binding of the engineered polynucleotide to the target sequence by base pairing such as Watson-Crick base pairing.

### Recruiting Moiety

As described in this disclosure, the engineered polynucleotides can comprise a recruiting moiety. The recruiting moiety can recruit one or more components of the spliceosome. In some embodiments, a recruiting moiety configured to recruit a spliceosomal moiety refers to a recruiting moiety that hybridizes to the spliceosomal moiety. In some embodiments, a recruiting moiety configured to recruit a spliceosomal moiety refers to a recruiting moiety that is at least 80%, 90%, or 100% complementary to the spliceosomal moiety or a sequence of the spliceosomal moiety. In some embodiments, “recruit” refers to formation of at least one hydrogen bond between the recruiting moiety and the spliceosomal moiety. In some embodiments, “recruit” refers to hybridization between at least one nucleotide of the recruiting moiety and at least one nucleotide of the spliceosomal moiety. A recruiting moiety comprises a hairpin structure. The hairpin may be a full hairpin or may be intercalated by internal loops. The hairpin structure may consist of 13 to 17 nucleotides. The recruiting moiety may interact with stem-loop II of the U1 snRNA. The recruiting moiety can comprise a sequence complementary to a sequence or portion of stem-loop II. For example, the stem-loop II of the U1 snRNA comprise the sequence 5′-GUAGGCCUCACGUUACCUAU-3′ (SEQ ID NO: 22), and the recruiting moiety may comprises 5′-CCGGA-3′. U1-A also can bind to stem-loop II of the U1 snRNA. Hydrogen bridges of stem-loop II with the hairpin/internal loop region can indirectly modulate polyadenylation and acetylation signaling by U1-A. The recruiting moiety may not silence the anchoring domain of U1-A in stem-loop II. This interaction can modulate gene expression and acetylation.

In some embodiments of the engineered polynucleotide described herein, the recruiting moiety comprises a nucleotide sequence that is at least 70%, 80%, 85%, or 90% identical or complementary to a sequence set forth in Table 2. In some embodiments, the recruiting moiety comprises a nucleotide sequence that is identical or complementary to a sequence set forth in Table 2. In some embodiments, the engineered polynucleotide comprises a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to any sequence selected from SEQ ID NOs: 1-2 In some embodiments, the engineered polynucleotide comprises a nucleotide sequence that is at least 70%, 80%, 85%, or 90% identical or complementary to any sequence selected from SEQ ID NOs: 1-2. In some embodiments, the engineered polynucleotide comprises a nucleotide sequence that is identical or complementary to any sequence selected from SEQ ID NOs: 1-2.

TABLE 2
Example recruiting moieties of engineered
polynucleotides described herein

| SEQ ID | | Nucleotide | |
|---|---|---|---|
| No. | Sequence | length | |
| 1 | ctaacctttcaggccag | 17 nt | ASMO1 (DNA) |
| 2 | cuaaccuuucaggccag | 17 nt | ASMO1 (RNA) |

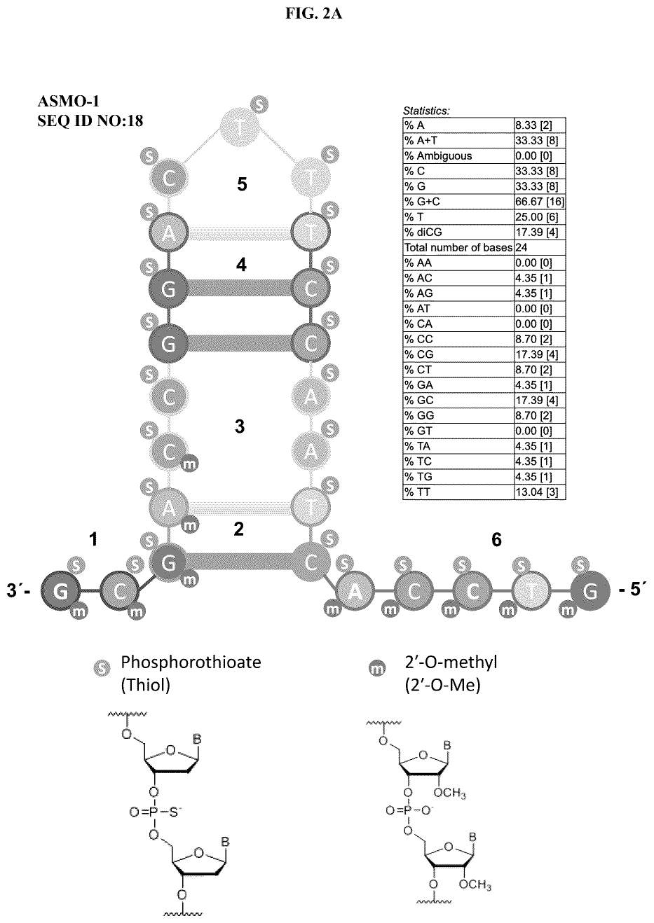

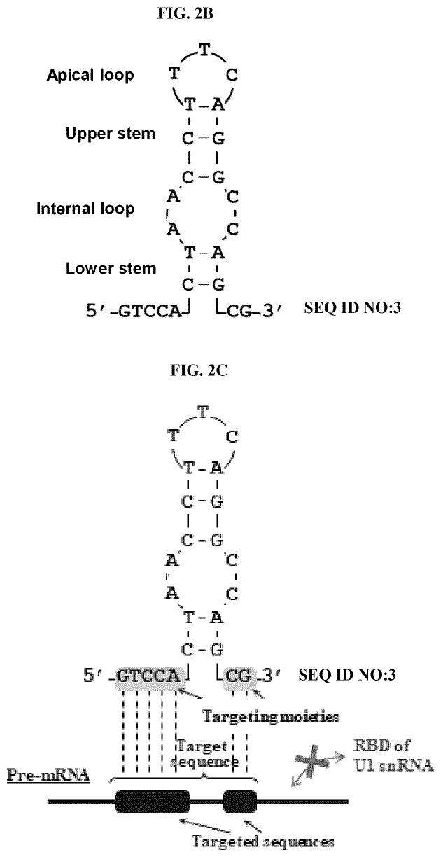

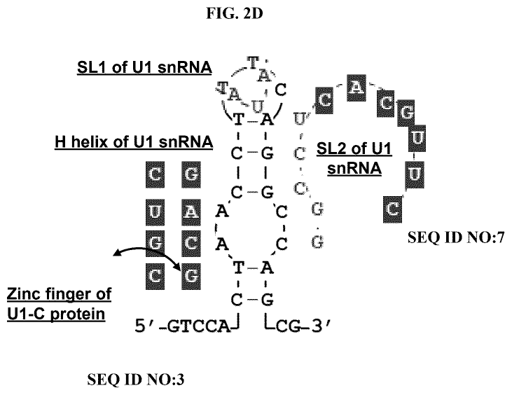

In some embodiments described herein, the engineered polynucleotide (e.g., the recruiting moiety) comprises a (e.g., secondary) structural feature (see FIGS. 2A-2D). In some embodiments, the engineered polynucleotide (e.g., the recruiting moiety) comprises an apical loop, an upper stem, an internal loop, a lower stem, or a combination thereof. In some embodiments, the engineered polynucleotide (e.g., the recruiting moiety) comprises a loop (e.g., an internal loop) adjacent to a stem (e.g., a lower stem or an upper stem) comprising two complementary stem sequences. In some embodiments, a stem sequence of the stem (e.g., the lower stem or the upper stem) comprises no more than about five, four, or three nucleotides. In some embodiments, the loop is an internal loop adjacent to the stem (e.g., the lower stem) and a further stem (e.g., an upper stem) comprising two complementary stem sequences. In some embodiments, the internal loop comprises a nucleic acid sequence of no more than 10, 9, or 8 nucleotides. In some embodiments, a stem sequence of the further stem (e.g., the upper stem) comprises no more than about five, four, or three nucleotides. In some embodiments, the engineered polynucleotide (e.g., the recruiting moiety) further comprises an apical loop. In some embodiments, the apical loop comprises a nucleic acid sequence of no more than 10, 9, 8, 7, 6, or 5 nucleotides.

In some embodiments of the engineered polynucleotide described herein, the recruiting moiety comprises about 10 to about 30 nucleotides, about 10 to about 25 nucleotides, or about 10 to about 20 nucleotides.

In some embodiments, the recruiting moiety is partially complementary to a post-transcriptional regulating moiety (or a regulating moiety) (e.g., a spliceosomal moiety) comprising a ribonucleoprotein complex. For example, the recruiting moiety can be partially complementary to a regulating moiety comprising a spliceosomal ribonucleoprotein complex, where the spliceosomal ribonucleoprotein complex comprises a small nuclear ribonucleic acid (snRNA). In some embodiments, the recruiting moiety is not complementary and does not bind to a target sequence described herein. For example, the recruiting moiety is not complementary and does not bind to a pre-mRNA described herein.

### Structural Configurations

In various aspects, the engineered polynucleotides comprise one or more moieties that can perform a function. These one or more moieties may be present in the engineered polypeptide in various structural configurations to allow for the engineered polynucleotide to perform a given function (e.g., recruitment of the regulating moiety or component of a spliceosome, or binding to a pre-mRNA). In some embodiments of the engineered polynucleotide described herein, one of the first and second targeting moieties is 5′ with respect to the recruiting moiety, and the other of the first and second targeting moieties is 3′ with respect to the recruiting moiety.

In some embodiments, the engineered polynucleotide has a structural arrangement from 5′-terminus to 3′-terminus as follows: a first targeting moiety, a recruiting moiety, and a second targeting moiety. In some embodiments, the engineered polynucleotide has a structural arrangement from 5′-terminus to 3′-terminus as follows: a second targeting moiety, a recruiting moiety, and a first targeting moiety.

### Example Polynucleotides

In some embodiments of the engineered polynucleotide described herein, the engineered polynucleotide comprises about 10 to about 40 nucleotides, about 10 to about 35 nucleotides, about 10 to about 30 nucleotides, or about 10 to about 25 nucleotides. In some embodiments, the engineered polynucleotide comprises a length of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleotides. In some embodiments, the engineered polynucleotide comprises a length of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or less nucleotides. In some embodiments, the engineered polynucleotide comprises a length of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50 nucleotides, or a range between any two foregoing values.

In some embodiments of the engineered polynucleotide described herein, the recruiting moiety comprises a nucleotide sequence that is at least 70%, 80%, 85%, or 90% identical or complementary to a sequence set forth in Table 3. In some embodiments, the recruiting moiety comprises a nucleotide sequence that is identical or complementary to a sequence set forth in Table 3.

TABLE 3
Example sequences of engineered polynucleotides
comprising recruiting moieties
and targeting moieties

| SEQ ID | | Nucleotide | |
|---|---|---|---|
| No. | Sequence | length | |
| 3 | gtccactaacct | 24 nt | ASMO1 |
| | ttcaggccagcg | | |
| 4 | guccacuaaccu | 24 nt | ASMO1 |
| | uucaggccagcg | | |

In some embodiments, the engineered polynucleotide can be produced from a precursor of the engineered polynucleotide. In some cases, a precursor of the engineered polynucleotide can be linear. For example, a precursor of the engineered polynucleotide can be a linear polynucleotide transcribed from a plasmid. In another example, a precursor of the engineered polynucleotide can be constructed to be a linear polynucleotide with moieties such as a ribozyme moiety and a ligation moiety that allow for circularization of the engineered polynucleotide in a cell. The linear engineered polynucleotide with the ligation and ribozyme moieties can be transfected into a cell, where it can be circularized. In some cases, the engineered polynucleotide can be circular. In some cases, the engineered polynucleotide comprises DNA, RNA or both. In some cases, a precursor of the engineered polynucleotide comprises a precursor of the engineered polynucleotide. In some cases, a precursor of the engineered polynucleotide can be used to produce an engineered polynucleotide.

In some embodiments, the engineered polynucleotide comprises at least one secondary structure (such as those described anywhere herein). For example, the engineered polynucleotide comprises at least one, two, three, four, or more secondary structures, where the secondary structures can be any one or any combination of an apical loop, a stem, a stem loop, or an internal loop. The one or more secondary structures may perform a variety of different functions. For example, the secondary structure may provide stability, or otherwise help stabilize the engineered polynucleotide. The secondary structure may bind to, or interact with polypeptide or other polynucleotide. For example, the secondary structure may interact with a component of the spliceosome.

In some embodiments, the recruiting moiety of the polynucleotide comprises at least one, two, three, four, or more secondary structures. In some embodiments, the targeting moiety does not have a secondary structure. In some embodiments, the secondary structure is an apical loop comprising at least two, three, four, five, six, seven, eight, nine, 10, or more nucleotides. In some embodiments, the apical loop is complementary and binds to the regulating moiety. In some embodiments, the apical loop is not complementary and does not bind to the regulating moiety. In some embodiments, the secondary structure is at least one stem. In some embodiments, the engineered polynucleotide comprises two stems, where one is an upper stem that is close to the apical loop and the other is a lower stem closer to the targeting moiety. In some embodiments, the upper stem comprises at least two, four, six, eight, 10, or more nucleotides, where the nucleotides are paired to form the upper stem. In some embodiments, the upper stem is complementary and binds to the regulating moiety. In some embodiments, the upper stem is not complementary and does not bind to the regulating moiety. In some embodiments, the secondary structure is a lower stem, where the lower stem comprises at least two, four, six, eight, 10, or more nucleotides, where the nucleotides are paired to form the lower stem. In some embodiments, the lower stem is complementary and binds to the regulating moiety. In some embodiments, the lower stem is not complementary and does not bind to the regulating moiety. In some embodiments, the engineered polynucleotide comprises an internal loop between the upper stem and the lower stem. In some embodiments, the internal loop comprises at least two, three, four, five, six, seven, eight, nine, 10, or more nucleotides. In some embodiments, the internal loop is complementary and binds to the regulating moiety. In some embodiments, the internal loop is not complementary and does not bind to the regulating moiety. In some embodiments, the engineered polynucleotide comprises secondary structures comprising an apical loop, an upper stem, an internal loop, and a lower stem, where the upper stem and the internal loop are at least partially complementary and bind to the regulating moiety. In some embodiments, the upper stem and the internal loop are complementary and bind to the regulating moiety comprising snRNA. In some embodiments, the snRNA is U1 snRNA such as US-A snRNA. In some embodiments, the snRNA is U2 snRNA.

In some embodiments, the nucleic acid sequence of the at least one secondary structure is partially complementary to the regulating moiety comprising the ribonucleoprotein complex. In some embodiments, the nucleic acid sequence of the at least one secondary structure is not complementary to the targeted nucleic acid sequence. In some embodiments, the engineered polynucleotide comprises at least one secondary structure of the nucleic acid. In some embodiments, the engineered polynucleotide comprises at least one, two, three, four, or more secondary structure of the nucleic acid. In some embodiments, the at least one secondary structure increases the binding between the recruiting moiety and the regulating moiety. In some embodiments, the at least one secondary structure stabilizes the assembly of the regulating moiety. In some embodiments, the at least one secondary structure stabilizes the assembly of the regulating moiety with other additional moiety. In some embodiments, the at least one secondary structure increases the efficiency of modulating the expression or activity of the gene encoding by the target sequence. In some embodiments, the at least one secondary structure increases the specificity of modulating the expression or activity of the gene encoding by the target sequence. In some embodiments, the at least one secondary structure increases the resistance of the engineered polynucleotide to degradation by hydrolysis. In some embodiments, the at least one secondary structure increases the resistance of the engineered polynucleotide to degradation by nuclease digestion. In some embodiments, the at least one secondary structure increases half-life of the engineered polynucleotide. In some embodiments, the at least one secondary structure decreases immunogenicity induced by the engineered polynucleotide.

In some embodiments, the engineered polynucleotide is characterized by a secondary structure. In some embodiments, the secondary structure comprises one or more stem-loop structures. In some embodiments, the secondary structure comprises an apical loop, an upper stem, an internal loop, and a lower stem. In some embodiments, the engineered polynucleotide comprises at least one secondary structure. In some embodiments, the first or second targeting moiety is not part of the secondary structure. In some embodiments, the engineered polynucleotide comprises at least one, two, three, four, or more secondary structure. In some embodiments, the engineered polynucleotide comprises a secondary structure comprising a stem-loop, a cruciform, a toe hold, a mismatch bulge, or any combination thereof. In some embodiments, the engineered polynucleotide comprises a secondary structure comprising an apical loop, an upper stem, an internal loop, or a lower stem. In some embodiments, the engineered polynucleotide comprises a secondary structure comprising an apical loop, an upper stem, an internal loop, and a lower stem. In some cases, a secondary structure can comprise a stem, a hairpin loop, a pseudoknot, a bulge, an internal loop, a multiloop, a G-quadruplex, or any combination thereof. In some embodiments, an engineered polynucleotide can adopt an A-form, a B-form, a Z-form, or any combination thereof. In some embodiments, the secondary structure is formed based on, at least partially, the nucleotide sequence of the engineered polynucleotide. In some embodiments, the secondary structure is formed within the nucleotide sequence of the engineered polynucleotide.

In some embodiments, the at least one secondary structure increases the binding between the recruiting moiety and the regulating moiety. In some embodiments, the at least one secondary structure increases the binding between the recruiting moiety and the regulating moiety by at least 10%, 20%, 30%, 40%, 50%, 60%, 70%, 80%, 90%, 2 fold, 3 fold, 4 fold, 5 fold, 10 fold, or more compared to the binding between the recruiting moiety without the secondary structure to the regulating moiety.

In some embodiments, the at least one chemical modification increases the binding between the recruiting moiety and the regulating moiety. In some embodiments, the at least one chemical modification increases the binding between the recruiting moiety and the regulating moiety by at least 10%, 20%, 30%, 40%, 50%, 60%, 70%, 80%, 90%, 2 fold, 3 fold, 4 fold, 5 fold, 10 fold, or more compared to the binding between the recruiting moiety without the chemical modification to the regulating moiety.

In some embodiments, the at least one chemical modification of the engineered polynucleotide stabilizes the assembly of the spliceosome comprising the regulating moiety, when the regulating moiety is associated with the target sequence. In some embodiments, the assembly of the spliceosome comprising the regulating moiety is at least 10%, 20%, 30%, 40%, 50%, 60%, 70%, 80%, 90%, 2 fold, 3 fold, 4 fold, 5 fold, 10 fold, or more stabilized by the engineered polynucleotide comprising the chemical modification compared to a comparable polynucleotide without the chemical modification. In some embodiments, the regulating moiety is U1 (U1SNP), U2 (U2SNP), U4, U5, U6, U11, U12, U14, or U16 of the spliceosome. In some embodiments, the regulating moiety is U1SNP of the spliceosome. In some embodiments, the regulating moiety is U1-A of the spliceosome. In some embodiments, the regulating moiety is U2SNP of the spliceosome. In some embodiments, the at least one chemical modification of the engineered polynucleotide stabilizes the assembly of the spliceosome comprising the regulating moiety and at least one additional moiety. For example, the at least one chemical modification of the engineered polynucleotide stabilizes the assembly of the spliceosome comprising the regulating moiety comprising U1-A and at least one additional moiety comprising U1-70K, UC-1, SmD1, SmD2, SmD3, SmE, SmF, or SmG. In some embodiments, the at least one additional moiety is U4, U5, U6, U11, U12, U14, or U16 of the spliceosome.

In some embodiments, the at least one chemical modification increases the efficiency of the engineered polynucleotide modulating the expression or activity of the gene encoding by the target sequence compared to a comparable polynucleotide without the chemical modification. In some embodiments, the efficiency of the engineered polynucleotide modulating the expression or activity of the gene encoding by the target sequence is increased by at least 10%, 20%, 30%, 40%, 50%, 60%, 70%, 80%, 90%, 2 fold, 3 fold, 4 fold, 5 fold, 10 fold, or more compared to the efficient of the comparable polynucleotide without the chemical modification modulating the expression or activity of the gene encoding by the target sequence.

In some embodiments, the at least one chemical modification increases the specificity of the engineered polynucleotide modulating the expression or activity of the gene encoding by the target sequence compared to a comparable polynucleotide without the chemical modification. In some embodiments, the specificity of the engineered polynucleotide modulating the expression or activity of the gene encoding by the target sequence is increased by at least 10%, 20%, 30%, 40%, 50%, 60%, 70%, 80%, 90%, 2 fold, 3 fold, 4 fold, 5 fold, 10 fold, or more compared to the efficient of the comparable polynucleotide without the chemical modification modulating the expression or activity of the gene encoding by the target sequence.

In some embodiments, the at least one chemical modification increases the resistance of the engineered polynucleotide to degradation by hydrolysis (e.g., degradation via an endonuclease). In some embodiments, the at least one chemical modification increases the resistance of the engineered polynucleotide to degradation by hydrolysis compared to a resistance of a comparable engineered polynucleotide without the chemical modification. In some embodiments, the resistance to degradation by hydrolysis of the engineered polynucleotide comprising the at least one chemical modification is increased by at least 10%, 20%, 30%, 40%, 50%, 60%, 70%, 80%, 90%, 2 fold, 3 fold, 4 fold, 5 fold, 10 fold, or more compared to the resistance of the comparable engineered polynucleotide without the chemical modification.

In some embodiments, the at least one chemical modification increases the resistance of the engineered polynucleotide to degradation by nuclease digestion. In some embodiments, the at least one chemical modification increases the resistance of the engineered polynucleotide to degradation by nuclease digestion compared to a resistance of a comparable engineered polynucleotide without the chemical modification. In some embodiments, the resistance to degradation by nuclease digestion of the engineered polynucleotide comprising the at least one chemical modification is increased by at least 10%, 20%, 30%, 40%, 50%, 60%, 70%, 80%, 90%, 2 fold, 3 fold, 4 fold, 5 fold, 10 fold, or more compared to the resistance of the comparable engineered polynucleotide without the chemical modification.

In some embodiments, the at least one chemical modification increases half-life of the engineered polynucleotide compared to a half-life of a comparable engineered polynucleotide without the chemical modification. In some embodiments, the half-life of the engineered polynucleotide comprising the at least one chemical modification is increased by at least 10%, 20%, 30%, 40%, 50%, 60%, 70%, 80%, 90%, 2 fold, 3 fold, 4 fold, 5 fold, 10 fold, or more compared to the half-life of the comparable engineered polynucleotide without the chemical modification.ne chemical modification increases half-life of the engineered polynucleotide.

In some embodiments, the at least one chemical modification decreases immunogenicity induced by the engineered polynucleotide. In some embodiments, the at least one chemical modification decreases immunogenicity induced by the engineered polynucleotide compared to an immunogenicity of a comparable engineered polynucleotide without the chemical modification. In some embodiments, the immunogenicity of the engineered polynucleotide comprising the at least one chemical modification is decreased by at least 10%, 20%, 30%, 40%, 50%, 60%, 70%, 80%, 90%, 2 fold, 3 fold, 4 fold, 5 fold, 10 fold, or more compared to the immunogenicity of the comparable engineered polynucleotide without the chemical modification.

### Chemical Modification(s)

In some embodiments described herein, the engineered polynucleotide comprises at least one chemical modification. As described in this disclosure, a chemical modification may confer structural or functional advantages to the engineered polynucleotides (e.g., increased half-life, decreased immunogenicity, increased resistance to hydrolysis or enzymatic degradation, improved reactivity or binding to a polypeptide (e.g., components of a spliceosome) or polynucleotide (e.g, pre-mRNA, or components of a spliceosome).

In some embodiments, all nucleotides of a targeting moiety are linked by phosphorothioate bonds. In some embodiments, all nucleotides of a targeting moiety comprise a 2′ O-methyl modification. 2′ modifications can prevent nuclease degradation and/or increase the affinity of the targeting moiety to a pre-mRNA target.

In some embodiments, all nucleotides of a recruiting moiety are linked by phosphorothioate bonds. In some embodiments, three nucleotides of a recruiting moiety comprise a 2′ O-methyl modification. The 2′ modifications can induce a change in the molecular dynamics of the recruiting moiety, thereby facilitating a conformational alteration of stem-loop II of U1-snRNA and binding of the recruiting moiety to U1-snRNA.

In some embodiments, the engineered polynucleotide comprises at least one 2′-modified (e.g., 2′-methoxy, 2′-methoxymethyl, or 2′-methoxyethyl) nucleotides. In some embodiments, at least about 50%, 60%, 70%, 80%, or 90% nucleotides of the engineered polynucleotide are chemically modified nucleotides. In some embodiments, at least about 50%, 60%, 70%, 80%, or 90% nucleotides of the engineered polynucleotide are 2′-modified (e.g., 2′-methoxy, 2′-methoxymethyl, or 2′-methoxyethyl) nucleotides. In some embodiments, the engineered polynucleotide comprises at least one phosphorothioate internucleotide bond. In some embodiments, at least about 50%, 60%, 70%, 80%, or 90% internucleotide linkages of the engineered polynucleotide are chemically modified. In some embodiments, at least about 50%, 60%, 70%, 80%, or 90% internucleotide linkages are phosphorathioate.

In some embodiments, the engineered polynucleotide comprises at least one chemical modification of the nucleic acid. In some embodiments, the engineered polynucleotide comprises at least one, two, three, four, or more chemical modification of the nucleic acid. In some embodiments, the at least one chemical modification increases the binding between the recruiting moiety and the regulating moiety. In some embodiments, the at least one chemical modification stabilizes the assembly of the regulating moiety. In some embodiments, the at least one chemical modification stabilizes the assembly of the regulating moiety with other additional moiety. In some embodiments, the at least one chemical modification increases the efficiency of modulating the expression or activity of the gene encoding by the target sequence. In some embodiments, the at least one chemical modification increases the specificity of modulating the expression or activity of the gene encoding by the target sequence. In some embodiments, the at least one chemical modification increases the resistance of the engineered polynucleotide to degradation by hydrolysis. In some embodiments, the at least one chemical modification increases the resistance of the engineered polynucleotide to degradation by nuclease digestion. In some embodiments, the at least one chemical modification increases half-life of the engineered polynucleotide. In some embodiments, the at least one chemical modification decreases immunogenicity induced by the engineered polynucleotide.

In some embodiments, the chemical modifications of the engineered polynucleotide comprise at least one substitution of one or both of non-linking phosphate oxygen atoms in a phosphodiester backbone linkage of the engineered polynucleotide. In some embodiments, the at least one chemical modification of the engineered polynucleotide comprises a substitution of one or more of linking phosphate oxygen atoms in a phosphodiester backbone linkage of the engineered polynucleotide. A non-limiting example of a chemical modification of a phosphate oxygen atom is a sulfur atom. Additional non-limiting examples are included in Table 3. In some embodiments, the chemical modifications of the engineered polynucleotide comprise at least one chemical modification to a sugar of a nucleotide of the engineered polynucleotide. In some embodiments, the chemical modifications of the engineered polynucleotide comprise at least one chemical modification to the sugar of the nucleotide, where the chemical modification comprises at least one locked nucleic acid (LNA). In some embodiments, the chemical modifications of the engineered polynucleotide comprise at least one chemical modification to the sugar of the nucleotide of the engineered polynucleotide comprising at least one unlocked nucleic acid (UNA). In some embodiments, the chemical modifications of the engineered polynucleotide comprise at least one chemical modification to the sugar comprising a modification of a constituent of the sugar, where the sugar is a ribose sugar. In some embodiments, the chemical modifications of the engineered polynucleotide comprise at least one chemical modification to the constituent of the ribose sugar of the nucleotide of the engineered polynucleotide comprising a 2′-O-Methyl group. In some embodiments, the chemical modification, instead of 2′-O-Methyl group modification, comprises a 2′-F-RNA. In such case, the 2′-F-RNA and pre-mRNA duplexes does not activate RNase H (degradation by nuclease digestion) and are more stable as determined by higher melting temperature (Tm) than 2′-O-methyl-RNA and pre-mRNA duplex.

In some embodiments, the chemical modifications of the engineered polynucleotide comprise at least one chemical modification comprising replacement of a phosphate moiety of the engineered polynucleotide with a dephospho linker. In some embodiments, the chemical modifications of the engineered polynucleotide comprise at least one chemical modification of a phosphate backbone of the engineered polynucleotide. In some embodiments, the engineered polynucleotide comprises a phosphothioate group. In some embodiments, the chemical modifications of the engineered polynucleotide comprise at least one chemical modification comprising a modification to a base of a nucleotide of the engineered polynucleotide. In some embodiments, the chemical modifications of the engineered polynucleotide comprise at least one chemical modification comprising an unnatural base of a nucleotide. In some embodiments, the chemical modifications of the engineered polynucleotide comprise at least one chemical modification comprising a morpholino group, a cyclobutyl group, pyrrolidine group, or peptide nucleic acid (PNA) nucleoside surrogate. In some embodiments, the chemical modifications of the engineered polynucleotide comprise at least one chemical modification comprising at least one stereopure nucleic acid. In some embodiments, the at least one chemical modification can be positioned proximal to a 5′ end of the engineered polynucleotide. In some embodiments, the at least one chemical modification can be positioned proximal to a 3′ end of the engineered polynucleotide. In some embodiments, the at least one chemical modification can be positioned proximal to both 5′ and 3′ ends of the engineered polynucleotide.

In some embodiments, the at least one chemical modification of the engineered polynucleotide comprises a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; Replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Example chemical modification to the engineered polynucleotide can be seen in Table 4.

TABLE 4
Example Chemical Modification

| Modification of | |
|---|---|
| engineered polynucleotide | Example(s) |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or replacement | Nucleic acid analog |
| of a naturally occurring | |
| nucleobase | |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

### Modification of Phosphate Backbone

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the engineered polynucleotide comprises stereopure nucleotides comprising S conformation of phosphorothioate or R conformation of phosphorothioate. In some embodiments, the chiral phosphate product is present in a diastereomeric excess of 50%, 60%, 70%, 80%, 90%, or more. In some embodiments, the chiral phosphate product is present in a diastereomeric excess of 95%. In some embodiments, the chiral phosphate product is present in a diastereomeric excess of 96%. In some embodiments, the chiral phosphate product is present in a diastereomeric excess of 97%. In some embodiments, the chiral phosphate product is present in a diastereomeric excess of 98%. In some embodiments, the chiral phosphate product is present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleotide linkage; N3′ to P5′ phosphoramidate modification; boranophosphate DNA; prooligonucleotides; neutral internucleotide linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleotide linkages, mixed heteroatom and alkyl or cycloalkyl internucleotide linkages, or one or more short chain heteroatomic or heterocyclic internucleotide linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the engineered polynucleotide described herein comprises at least one chemically modified phosphate backbone. Example chemically modification of the phosphate group or backbone can include replacing one or more of the oxygens with a different substituent. Furthermore, the modified nucleotide present in the engineered polynucleotide can include the replacement of an unmodified phosphate moiety with a modified phosphate as described herein. In some embodiments, the modification of the phosphate backbone can include alterations resulting in either an uncharged linker or a charged linker with unsymmetrical charge distribution. Example modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified engineered polynucleotide can be stereopure (e.g. S or R confirmation). In some cases, the chemically modified engineered polynucleotide comprises stereopure phosphate modification. For example, the chemically modified engineered polynucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered polynucleotide can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, the chemical modification described herein comprises modification by replacement of a phosphate group. In some embodiments, the engineered polynucleotide described herein comprises at least one chemically modification comprising a phosphate group substitution or replacement. Example phosphate group replacement can include non-phosphorus containing connectors. In some embodiments, the phosphate group substitution or replacement can include replacing charged phosphate group can by a neutral moiety. Example moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein comprises modifying ribophosphate backbone of the engineered polynucleotide. In some embodiments, the engineered polynucleotide described herein comprises at least one chemically modified ribophosphate backbone. Example chemically modified ribophosphate backbone can include scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein comprises modifying of sugar. In some embodiments, the engineered polynucleotide described herein comprises at least one chemically modified sugar. Example chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH2CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where example bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the Γ position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the engineered polynucleotide described herein includes the sugar group ribose, which is a 5-membered ring having an oxygen. Example modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some embodiments, the modifications to the sugar of the engineered polynucleotide comprises modifying the engineered polynucleotide to include locked nucleic acid (LNA), unlocked nucleic acid (UNA), or bridged nucleic acid (BNA).

### Modification of a Constituent of the Ribose Sugar

In some embodiments, the engineered polynucleotide described herein comprises at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH2-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the engineered polynucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)n CH₃)]₂, where n and m are from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some embodiments, the chemical modification described herein comprises modification of the base of nucleotide (e.g. the nucleobase). Example nucleobases can include adenine (A), thymine (T), guanine (G), cytosine (C), and uracil (U). These nucleobases can be modified or replaced to in the engineered polynucleotide described herein. The nucleobase of the nucleotide can be independently selected from a purine, a pyrimidine, a purine or pyrimidine analog. In some embodiments, the nucleobase can be naturally occurring or synthetic derivatives of a base.

In some embodiments, the chemical modification described herein comprises modifying an uracil. In some embodiments, the engineered polynucleotide described herein comprises at least one chemically modified uracil. Example chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxy carbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, l-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, l-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein comprises modifying a cytosine. In some embodiments, the engineered polynucleotide described herein comprises at least one chemically modified cytosine. Example chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying a adenine. In some embodiments, the engineered polynucleotide described herein comprises at least one chemically modified adenine. Example chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the engineered polynucleotide described herein comprises at least one chemically modified guanine. Example chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, O⁶-Methyl-2′-deoxyguanosine, 2′-F-ara-guanosine, and 2′-F-guanosine.

In some cases, the chemical modification of the engineered polynucleotide can include introducing or substituting a nucleic acid analog or an unnatural nucleic acid into the engineered polynucleotide. In some embodiments, nucleic acid analog can be any one of the chemically modified nucleic acid described herein. Example nucleic acid analog can be found in PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Example chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2,—O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-TAUrinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered polynucleotide. In some embodiments, the engineered polynucleotide comprises a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

In some embodiments, all nucleotides of a targeting moiety have 2′ O-methyl modifications. The 2′ O-methyl modification is thought to increase the affinity of an engineered polynucleotide for its pre-mRNA targets and/or prevent degradation of an engineered polynucleotide by nucleases. In some embodiments, all nucleotides of a targeting moiety have phosphorothioate modifications.

### Regulating Moiety

In some embodiments of the engineered polynucleotide described herein, the post-transcriptional regulating moiety (or the regulating moiety) (e.g., the spliceosomal moiety) is selected from a spliceosomal ribonucleoprotein complex, a spliceosomal small nuclear ribonucleic acid (snRNA), a spliceosomal protein, a functional variant thereof, or a functional fragment thereof. In some embodiments, the spliceosomal moiety comprises U1 snRNA and a spliceosomal protein. In some embodiments, the spliceosomal moiety comprises U2 snRNA and a spliceosomal protein. In some embodiments, the spliceosomal snRNA is selected from U1, U2, U4, U5, U6, U11, U12, U14atac, U6atac, and combinations thereof. In some embodiments, the spliceosomal snRNA is U1 or U2. In some embodiments, the spliceosomal protein is selected from Sm, U1-70k, U1A, U1C, and combinations thereof. Non-limiting example of the spliceosomal moiety includes SmD1, SmD2, SmD3, SmE, SmF, SmG, U1, U2, U4, U5, U6, U11, U12, U14, or U16.

In some embodiments described herein, when associated with the engineered polynucleotide and the RNA (e.g., the mRNA, such as the pre-mRNA), the spliceosomal moiety cleaves or splices the RNA (e.g., the mRNA, such as the pre-mRNA) in the target sequence. In some embodiments, the spliceosomal moiety further facilitates modification of a cleaved RNA (e.g., a cleaved mRNA, such as a cleaved pre-mRNA).

In some embodiments, the binding of the engineered polynucleotide to the target sequence by base pairing such as Watson-Crick base pairing. The binding of the engineered polynucleotide to the recruiting moiety provided herein can be utilized to modulate expression or activity of a target gene. In some embodiments, the binding of the engineered polynucleotide to the recruiting moiety enables the recruiting moiety to splice the pre-mRNA encoding the target gene with increased specificity, thus modulating the target gene. In some embodiments, the binding of the engineered polynucleotide to the recruiting moiety enables the recruiting moiety to splice the pre-mRNA encoding the target gene with increased efficiency, thus modulating the target gene. Modulation can refer to increasing or decreasing the expression or activity of the target gene. Non-limiting example of the target gene can include microtubule associated protein TAU (MAPT). In some embodiments, the expression or activity of the target gene, when the engineered polynucleotide binds to the recruiting moiety, is increased by at least 10%, 20%, 30%, 40%, 50%, 60%, 70%, 80%, 90%, 2 fold, 3 fold, 4 fold, 5 fold, 10 fold, or more compared to the expression or activity of the target gene in the absence of the engineered polynucleotide binding to the recruiting moiety. In some embodiments, the expression or activity of the target gene, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the expression or activity of the target gene in the absence of the engineered polynucleotide binding to the recruiting moiety.

In some embodiments, the modulating of the expression or activity of the target comprises correcting aberrant expression of the target gene due to splice variant. In some embodiments, the expression or activity of misfolded target gene or protein due to aberrant splice variant, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the expression or activity of misfolded target gene or protein due to aberrant splice variant in the absence of the engineered polynucleotide binding to the recruiting moiety. In some embodiments, the amount of misfolded protein aggregate due to aberrant splice variant, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the amount of misfolded protein aggregate due to aberrant splice variant in the absence of the engineered polynucleotide binding to the recruiting moiety. In some embodiments, the amount of plaques comprising the misfolded protein due to aberrant splice variant, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the amount of plaques comprising the misfolded protein due to aberrant splice variant in the absence of the engineered polynucleotide binding to the recruiting moiety.

### Molecular Interaction(s)

In various aspects of the disclosure, engineered polynucleotides may be able to participate in molecular interactions with polypeptides (e.g., U1-C) or other polynucleotides (e.g., pre-mRNAs, U1 snRNA). The engineered polynucleotides may be configured to, or otherwise able to interact with other polypeptides or polynucleotides via one or more targeting moieties or recruiting moieties. In some embodiments described herein, a targeting moiety comprises free 5′ and 3′ ends that interact with a conserved site of a constitutive splice donor. This interaction can silence the U1 snRNA RNA-binding domain. In some embodiments, the targeting moiety comprises 2′ modified nucleotides that increase affinity to pre-mRNA targets.

In some embodiments, binding of a targeting moiety to a pre-mRNA is stabilized by hydrogen bonds and electrostatic interactions between U1-C and the pre-mRNA backbone around a splice junction region for the pre-mRNA. In such embodiments, U1-C may not make specific base contacts with the pre-mRNA. 2′ nucleotide modifications can favor hydrogen bonding between a targeting moiety and U1-C. Accordingly binding of a targeting moiety (free 5′ and 3′ ends) to a pre-mRNA duplex enables recognition and stabilization of the targeting interaction by U1-C

U1-C can stabilize the spliceosome central nucleus. U1-C enhances the affinities of incompatible 5′-splices and stabilizes the central core of the spliceosome machinery through an interaction bridge between U1-70KD and the Sm ring.

In some embodiments, a targeting moiety can interact with the zinc finger of U1-C. Phosphorothioate internucleotide linkages in the targeting moiety can promote interaction of a targeting moiety and the zinc finger. The engineered polynucleotides may comprise phosphorothioate internucleotide linkages at a specific or particular position that interacts with a zinc finger.

In some embodiments, a recruiting moiety forms hydrogen bonds to stem-loop II of U1-A. Such interactions can modulate polyadenylation and acetylation signaling by U1-A because stem-loop II of UT-A may not be silenced by the recruiting moiety.

In some embodiments described herein, the engineered polynucleotide does not comprise any intramolecular disulfide bond.

In some embodiments of the engineered polynucleotide described herein, when associated with the engineered polynucleotide and the spliceosomal moiety, the RNA (e.g., the mRNA, such as the pre-mRNA) exhibits substantially no base pairing with an RNA binding domain (RBD) of U1 snRNA.

In some embodiments of the engineered polynucleotide described herein, when associated with the engineered polynucleotide and the spliceosomal moiety, the RNA (e.g., the mRNA, such as the pre-mRNA) exhibits substantially no base-specific interaction with U1-C protein.

In some embodiments of the engineered polynucleotide described herein, the engineered polynucleotide is configured to specifically interact with zinc-finger of U1-C protein, e.g., comprising an amino acid sequence of YYCDYCDTYLTHDSPSVRKTHCTGRKHRDNVKF (SEQ ID NO: 5). In some embodiments, a 5′-targeting moiety of the engineered polynucleotide is configured to specifically interact with zinc-finger of U1-C protein. In some embodiments, (e.g., the 5′-targeting moiety of) the engineered polynucleotide is configured to covalently interact (e.g., via disulfide bonding) with zinc-finger of U1-C protein. In some embodiments, (e.g., the 5′-targeting moiety of) the engineered polynucleotide is configured to non-covalently interact (e.g., via hydrogen bonding) with zinc-finger of U1-C protein.

In some embodiments, the engineered polynucleotide (e.g., ASMO1 described herein) comprises a nucleotide sequence complementary to U1 snRNA. In some embodiments of the engineered polynucleotide described herein, the engineered polynucleotide comprises a nucleotide sequence complementary to a partial sequence of Stem-Loop II (SL2) of U1 snRNA. In some embodiments, a side of a stem-loop structure of the engineered polynucleotide comprises a nucleotide sequence complementary to a partial sequence of Stem-Loop II (SL2) of U1 snRNA. In some embodiments, the partial sequence comprises the sequence corresponding to 5′-GGCCU-3′ of SL2 of U1 snRNA. In some embodiments, the partial sequence does not comprise the sequence corresponding to 5′-CACGUUA-3′ of SL2 of U1 snRNA.

In some embodiments of the engineered polynucleotide described herein, the engineered polynucleotide exhibits substantially no base pairing with an anchoring sequence of SL2 of U1 snRNA. In some embodiments, the anchoring sequence comprises the sequence corresponding to 5′-CACGUUA-3′. In some embodiments, an internal loop of the engineered polynucleotide exhibits substantially no base pairing with the anchoring sequence of the SL2 of U1 snRNA. In some embodiments, a lower stem of the engineered polynucleotide exhibits substantially no base pairing with the anchoring sequence of the SL2 of U1 snRNA.

In some embodiments of the engineered polynucleotide described herein, the engineered polynucleotide exhibits substantially no base pairing with H helix of U1 snRNA.

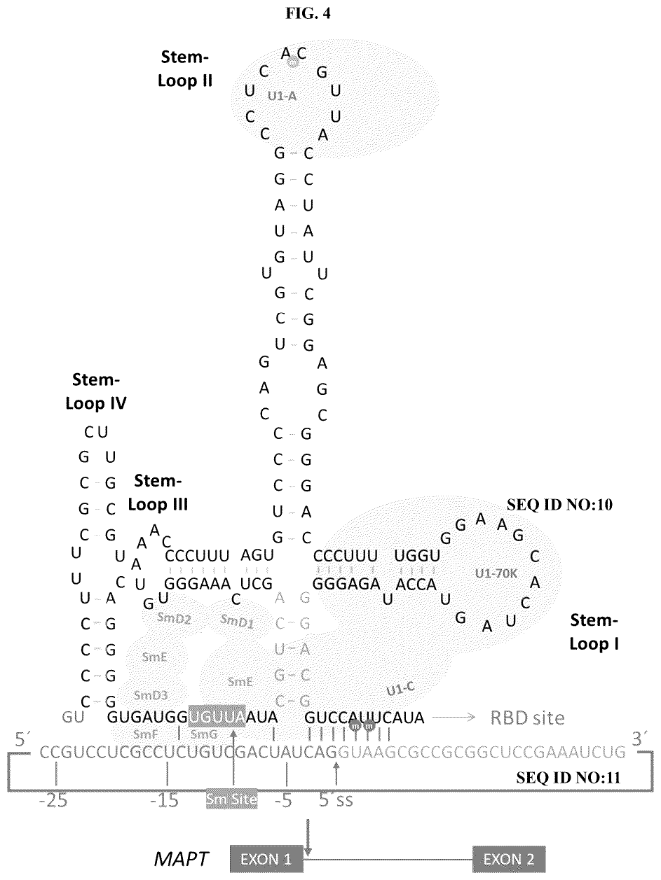

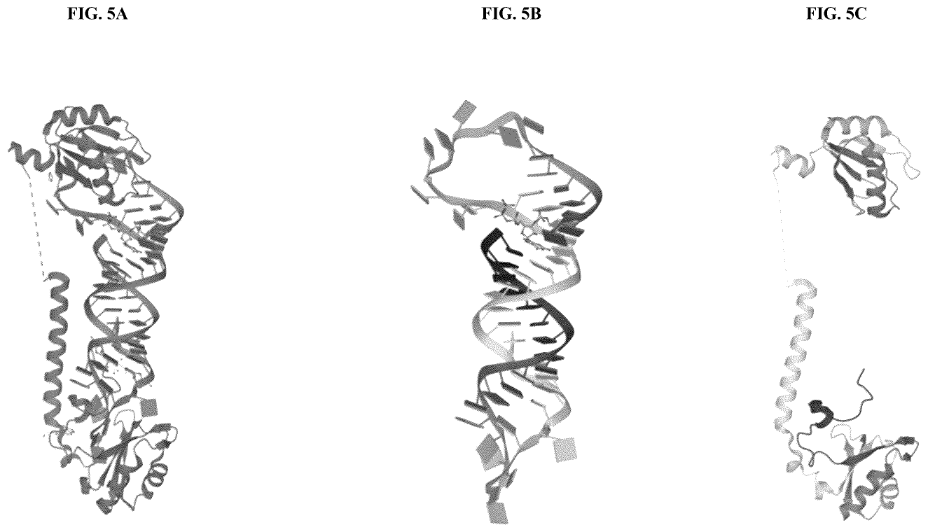

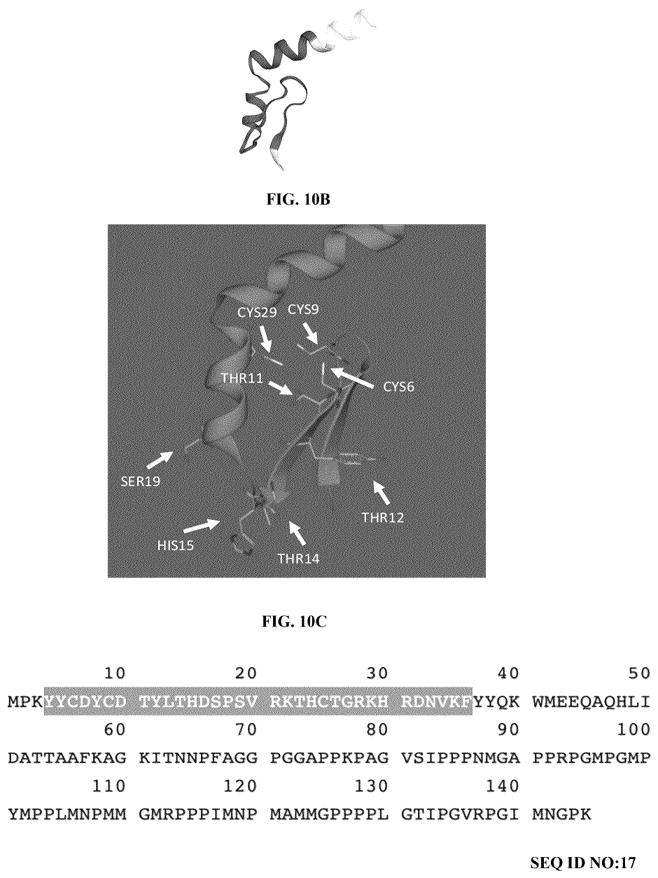

In some embodiments, the engineered polynucleotide does not comprise any intramolecular disulfide bond. In some embodiments, the engineered polynucleotide, when recruiting the spliceosomal moiety described to the target sequence such as target pre-mRNA, the engineered polynucleotide does not exhibit base pairing with an RNA binding domain (RBD) of the spliceosomal moiety such as U1 snRNA. FIG. 4 illustrates such lack of base pairing between the engineered polynucleotide and the RBD of the spliceosomal moiety, where the RBD site of U1 snRNA has the following sequence: 3′-GUCCAUUCAUA-5′ (SEQ ID NO: 6) and forms base pairing with the target sequence. In some instances, when the engineered polynucleotide and the spliceosomal moiety are bound, the engineered polynucleotide exhibits substantially no base-specific interaction with U1-C spliceosomal moiety. In some embodiments, the engineered polynucleotide is configured to specifically interact with zinc-finger of U1-C protein or U1-1 spliceosomal moiety. FIG. 10C illustrates a representative sequence of U1-C snRNP containing 145 amino acids, with the highlighted 36 amino acids (YYCDYCDTYLTHDSPSVRKTHCTGRKHRDNVKF (SEQ ID NO: 5)) comprising the zinc-finger domain.

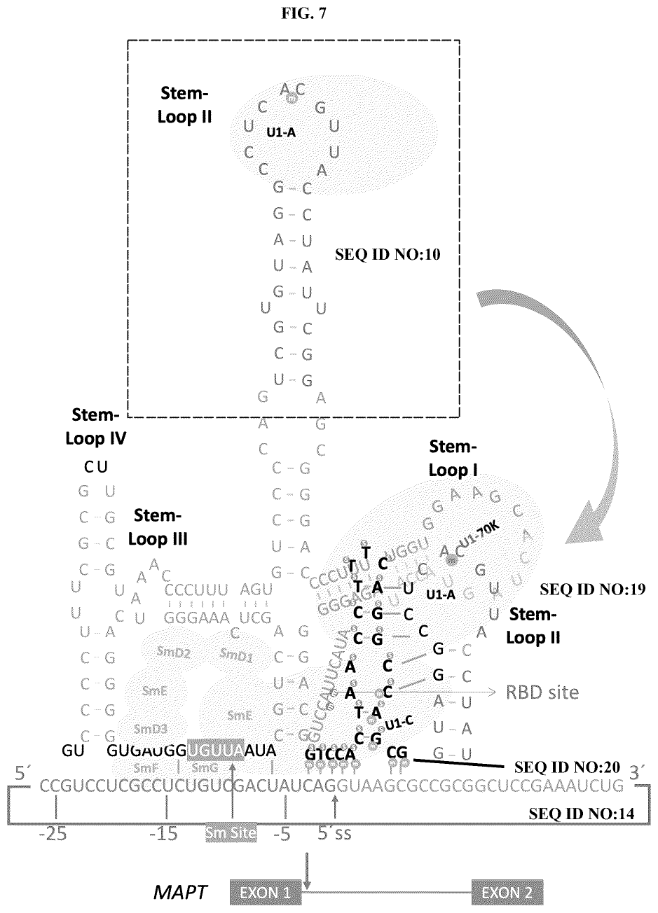

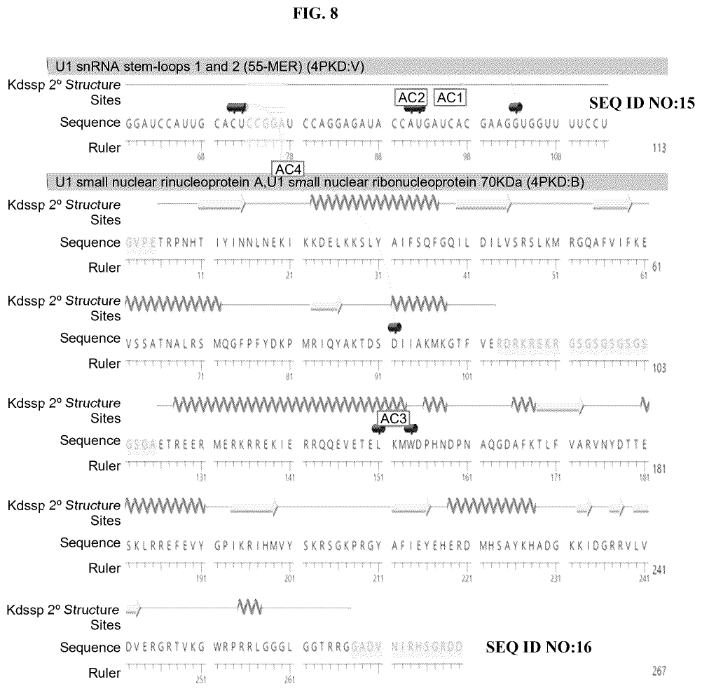

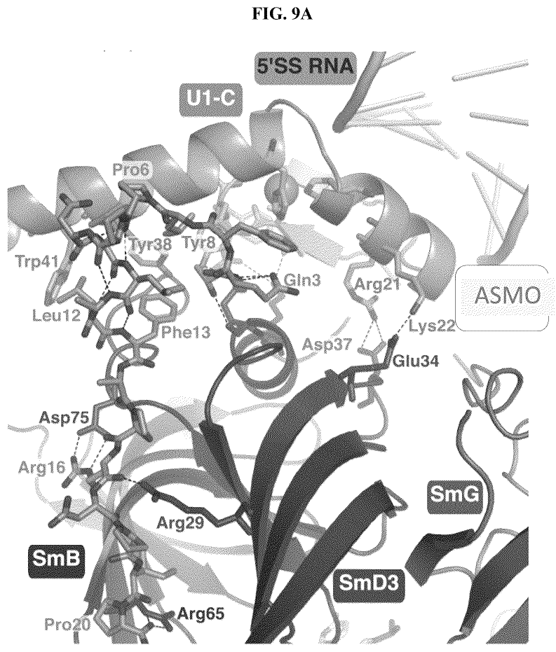

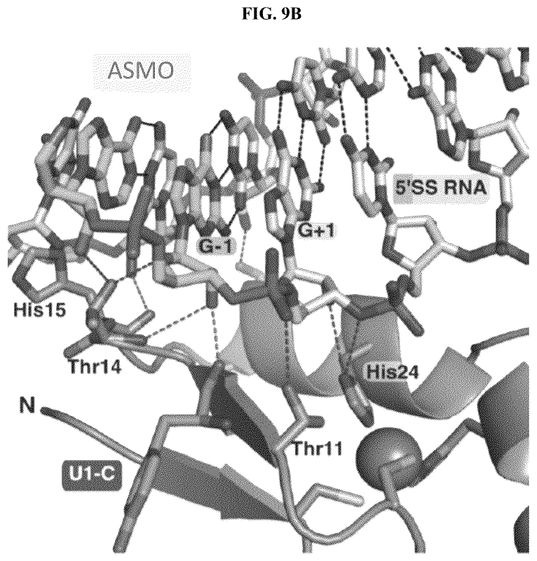

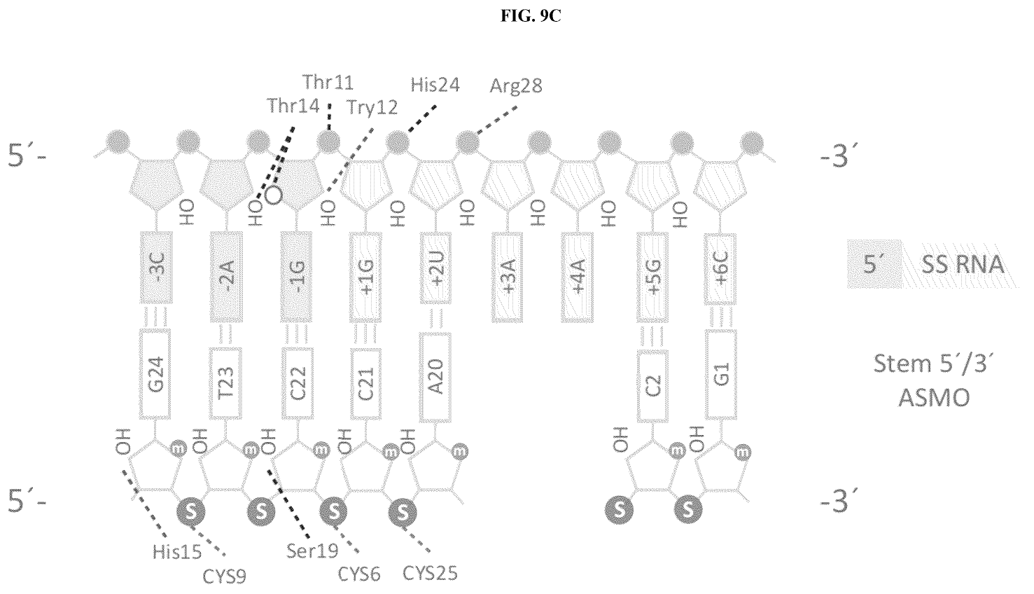

In some embodiments, the engineered polynucleotide is configured to covalently interact (e.g., via disulfide bonding) with zinc-finger of U1-C protein or U1-1 spliceosomal moiety. In some embodiments, the engineered polynucleotide is configured to non-covalently interact (e.g., via hydrogen bonding) with zinc-finger of U1-C protein or U1-1 spliceosomal moiety. In some embodiments, the engineered polynucleotide (e.g., ASMO1 described herein) comprises a nucleotide sequence complementary to U1 snRNA. In some embodiments, the engineered polynucleotide comprises a nucleotide sequence complementary to a partial sequence of Stem-Loop II (SL2) of U1 snRNA. In some embodiments, a side of a stem-loop secondary structure of the engineered polynucleotide comprises a nucleotide sequence complementary to a partial sequence of Stem-Loop II (SL2) of U1 snRNA. In some embodiments, the partial sequence comprises the sequence corresponding to 5′-GGCCU-3′ of SL2 of U1 snRNA. In some embodiments, the partial sequence does not comprise the sequence corresponding to 5′-CACGUUA-3′ of SL2 of U1 snRNA. In some embodiments, the engineered polynucleotide exhibits substantially no base pairing with an anchoring sequence of SL2 of U1 snRNA. In some cases, the internal loop of the engineered polynucleotide exhibits substantially no base pairing with the anchoring sequence of the SL2 of U1 snRNA. In some aspects, the lower stem of the engineered polynucleotide exhibits substantially no base pairing with the anchoring sequence of the SL2 of U1 snRNA. In some aspects, the anchoring sequence comprises the sequence corresponding to 5′-CACGUUA-3′. In some cases, the engineered polynucleotide exhibits substantially no base pairing with H helix of U1 snRNA, where the engineered polynucleotide does not comprise any intramolecular disulfide bond. For example, FIG. 2A illustrates the lack of intramolecular disulfide bond due to the presence of chemical modification with phosphorothioate-type internucleotide bonds. Stabilization of the U1 snRNP complex can be observed through the strong ionic attraction of the Zinc Finger of U1-C, induced by disulfide bridges with thiol of the engineered polynucleotide (ASMO) targeting moieties at 5′- or/and 3′-end. The pre-mRNA/engineered polynucleotide (ASMO) duplex bond can be stabilized by hydrogen bonds and electrostatic interactions between U1-C and the backbone of the pre-mRNA around the seam joint, but U1-C does not make base-specific contacts with pre-mRNA. The structure demonstrates that the selection of nucleotides of 5′-splices by U1 snRNP is achieved predominantly through the interaction between Stem 5′/3′ with pre-mRNA. Meanwhile, U1-C adjusts relative affinities of incompatible sites of 5′-splices and stabilizes the central core of spliceosome machinery by the interaction bridge between U1-70KDa and the Sm ring (see FIGS. 7-9). Of the U1 snRNP specific proteins, U1-70k and U1-C have important roles in aiding recognition of the pre-mRNA transcript. U1-70k has an N-terminus that while highly conserved is predicted to be unstructured (residues ˜2-60), an RNA binding domain (or RBD) that mediates its interaction with a stem-loop of U1 snRNA (residues 92-202), and a C-terminus rich in repeats of arginine and serine residues (an RS ‘domain’) as well as R-(D/E) residues. Although this C-terminal domain is not conserved the RS ‘domain’ is important for interaction with non-snRNP splicing factors such as ASF/SF2. Serines in this region are subjected to post-translational modification (phosphorylation) and are important to splicing activity. U1-C consists of an N-terminal zinc-finger domain and a C-terminal region rich in repeats of RG residues. Arginines in this region of U1-C are subjected to post-translational modification (methylation). In contrast to U1-70k, U1-C does not bind to free U1 snRNA but requires the prior binding of the Sm proteins and U1-70k. Mutations in the zinc-finger region of U1-C have a significant effect on recognition of the 5′ splice site by the U1 snRNP, indicating that this protein has a direct role to play in this activity. The assembly and function of U1 snRNP has been greatly enhanced initially by cryo electron microscopy studies and more recently by elucidation of its three-dimensional structure by X-ray crystallography. Previously, crystal structures of four of seven Sm proteins led to the modeling of the remaining three (Sm-F, Sm-E and Sm-G) and the proposal that together they would interact to form a seven-membered ring. The crystal structure of a completely recombinant human U1 snRNP reveals that Sm proteins do form a heptameric ring, composed of a single copy of each Sm protein, and passing through its center is the Sm site of U1 snRNA. In the crystal structure, U1-C is in a position to recognize the duplex formed when the 5′ end of U1 snRNA base-pairs to the 5′ splice site. The finding that the N-terminus of U1-70k extends 180 Å from the RBD and wraps around one face of the Sm ring, crossing Sm-D2 and Sm-D3/B, could therefore ensure the correct structure and positioning of U1-C for interaction with the U1 snRNA:5′ splice site duplex (FIG. 9).

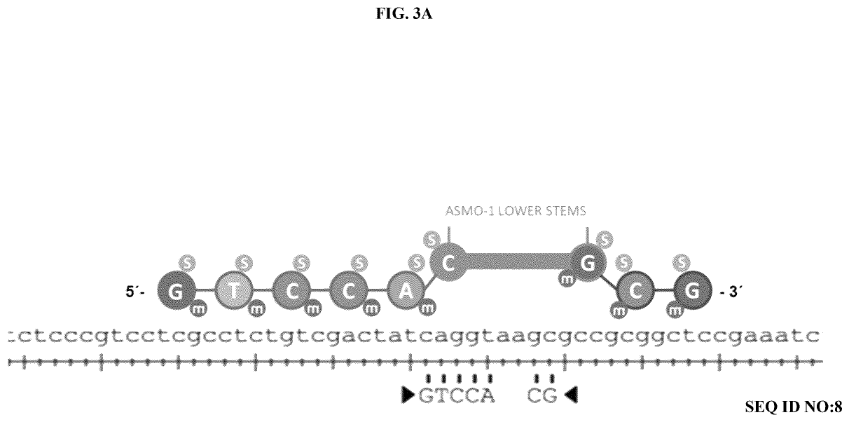

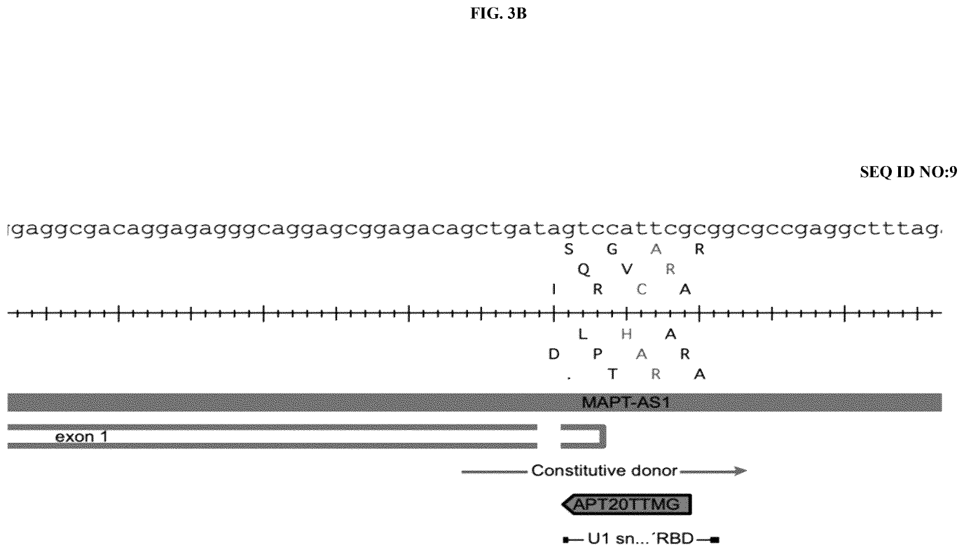

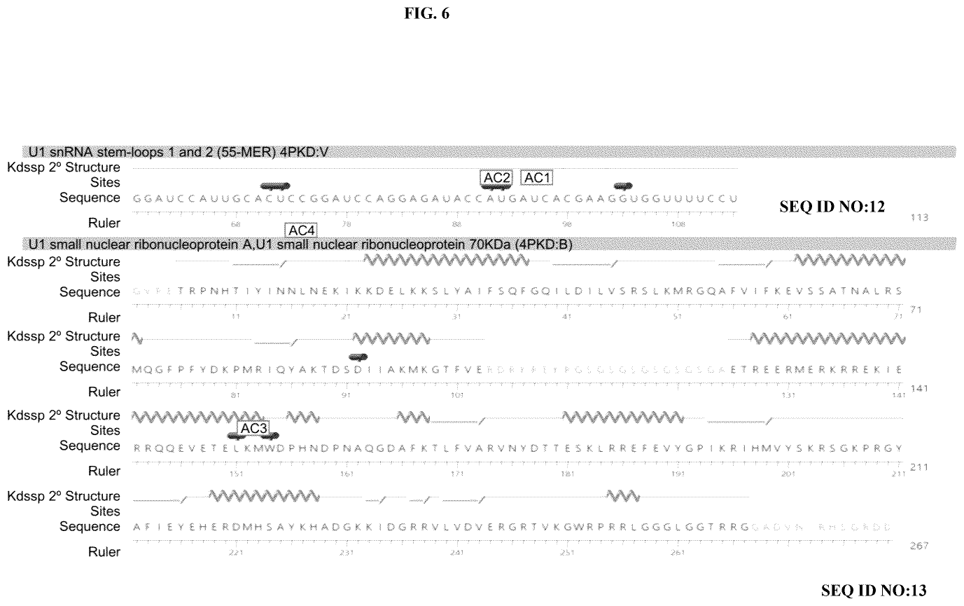

In some embodiments, the engineered polynucleotide, when associated with the spliceosomal moiety described herein, the pre-mRNA exhibits substantially no base pairing with an RNA binding domain (RBD) of U1 snRNA. In some cases, the engineered polynucleotide, when associated with the spliceosomal moiety, the pre-mRNA exhibits substantially no base-specific interaction with U1-C protein. FIG. 4 illustrates that in the absence of the engineered polynucleotide, the RBD of the U1 snRNA binds in the conserved regions of constitutive donor. Meanwhile, in the presence of the engineered polynucleotide the stem 5′/3′ blocked the RBD interaction of the U1 snRNA with the pre-mRNA (FIG. 3 and FIG. 6).

In some embodiments, the engineered polynucleotide is configured to specifically interact with zinc-finger of U1-C protein. In some embodiments, a 5′-targeting moiety of the engineered polynucleotide is configured to specifically interact with zinc-finger of U1-C protein. In some embodiments, the engineered polynucleotide is configured to covalently interact (e.g., via disulfide bonding) with zinc-finger of U1-C protein. In some embodiments, the engineered polynucleotide is configured to non-covalently interact (e.g., via hydrogen bonding) with zinc-finger of U1-C protein. The formation of the pre-mRNA/engineered polynucleotide (ASMO) duplex that interacts with amino acid residues from the U1-C zinc finger stabilizes the 5; region (FIG. 9). Then, favorable molecular dynamics for formation by disulfide bonds formed by atoms in the main and side chain of the U1-C zinc finger with Stem 5′ of the engineered polynucleotide can be observed. A strong ionic bond can also form as ASMO presents interaction with all the cysteines present in the U1-C zinc finger (FIG. 9 and FIG. 10). Additional example interaction between U1-C and pre-mRNA in the presence or in the absence of the engineered polynucleotide described herein is shown in Table 5.

TABLE 5
Example interaction between U1-C and pre-mRNA mediated
by the presence of the engineered polynucleotide

| | In presence of engineered | In absence of engineered |
|---|---|---|
| Domain | polynucleotide | polynucleotide |
| Constitutive | The binding of the 5′/3′ Stem allows | The presence of mutations in the |
| Donor 5′ | the interaction with the conserved site | RBD domain of U1-snRNA can |
| | of the constitutive donor, by silencing | induce a crypto splice or lack of |
| | the U1 snRNA RNA-binding domain | recognition of the 5′ region |
| | (RBD) | |
| Duplex | The pre-mRNA/engineered | Unfavorable molecular dynamics |
| recognition 5′ | polynucleotide duplex bond is | due to the low ionic attraction of the |
| by U1-C | stabilized by hydrogen bonds and | U1-C Zinc finger. It may induce a |
| | electrostatic interactions between U1- | crypto splice or instability in the |
| | C and the pre-mRNA backbone around | formation of the U1 complex in the |
| | the junction region, but U1-C does not | 5′ region |
| | make specific base contacts with pre- | |
| | mRNA. It is important to note that the | |
| | 2′-OME group favors the interaction | |
| | of hydrogen bonds with U1-C | |
| Stabilization | The pre-mRNA/engineered | Unfavorable molecular dynamics |
| of the | polynucleotide duplex bond is | due to the |
| 5′ duplex by | stabilized by hydrogen bonds and | low ionic attraction of the U1-C |
| U1-C | electrostatic interactions between U1- | Zinc finger. It may induce a crypto |
| | C and the pre-mRNA backbone around | |
| Domain | In presence of engineered | In absence of engineered |
| | polynucleotide | polynucleotide |
| | the junction region, but U1-C does not | splice or instability in the formation |
| | make specific base contacts with pre- | of the U1 complex in the 5′ region |
| | mRNA | |
| | The 2′-OME group favors the | |
| | interaction of hydrogen bonds with | |
| | U1-C | |
| Stabilization | U1-C adjusts high-precision relative | U1-C sits on SmD3 and its binding |
| of the | affinities of incompatible 5′-splices | is stabilized by the N-terminus of |
| spliceosome | and stabilizes the central core of | U1-70k |
| central | spliceosome machinery through the | |
| nucleus | interaction bridge between U1-70KDa | |
| | and the Sm ring | |
| Stem-loop | Hydrogen bridges of Stem-Loop II | SNRPA binds stem loop II of U1 |
| | with specific Hairpin-2 (a.k.a., upper | snRNA. In a snRNP-free form (SF- |
| | stem, see FIG. 2B) base and internal | A) may be involved in coupled pre- |
| | loop, associated with the modulation | mRNA splicing and |
| | of the polyadenylation and acetylation | polyadenylation process. May bind |
| | signal by U1-A. It is noted that the | preferentially to the 5′-UGCAC-3′ |
| | anchoring domain of U1-A in Stem- | motif on RNAs. The loss of U1-A |
| | Loop II, is not silenced by Hairpin-2, | self-regulation can induce |
| | inducing the modulation of levels of | premature poly (A) signal, |
| | gene expression and acetylation. The | dysregulation of gene expression |
| | presence of 2′-OME groups induces a | and inadequate acetylation. |
| | change in the molecular dynamics of | |
| | the medium facilitating the | |
| | conformational alteration of U1- | |
| | snRNA and approximation of Stem- | |
| | loop II to engineered polynucleotide. | |

In some embodiments, the engineered polynucleotide (e.g., ASMO1 described herein) comprises a nucleotide sequence complementary to UT snRNA. In some aspects, the engineered polynucleotide comprises a nucleotide sequence complementary to a partial sequence of Stem-Loop II (SL2) of UT snRNA. In other aspects, described herein is a side of a stem-loop structure of the engineered polynucleotide comprises a nucleotide sequence complementary to a partial sequence of Stem-Loop II (SL2) of UT snRNA. In some instances, the partial sequence comprises the sequence corresponding to 5′-GGCCU-3′ of 5SL2 of UT snRNA, where the partial sequence does not comprise the sequence corresponding to 5′-CACGUUA-3′ of 5SL2 of UT snRNA and where the engineered polynucleotide exhibits substantially no base pairing with an anchoring sequence of SL2 of UT snRNA. In some aspects, the engineered polynucleotide comprises an internal loop of the engineered polynucleotide exhibits substantially no base pairing with the anchoring sequence of the SL2 of UT snRNA. In some embodiments, the engineered polynucleotide comprises a lower stem of the engineered polynucleotide exhibiting substantially no base pairing with the anchoring sequence of the SL2 of U1 snRNA. In some embodiments, the anchoring sequence comprises the sequence corresponding to 5′-CACGUUA-3′, where then engineered polynucleotide exhibits substantially no base pairing with H helix of U1 snRNA.

### Set(s) of Engineered Polynucleotides

Described herein, in some embodiments, include a set of engineered polynucleotides each independently described herein. For example, the polynucleotides of the set may independently comprise: (i) one or more targeting moiety (such as described herein) configured to bind a ribonucleic acid (RNA) (such as described herein) (e.g., a messenger ribonucleic acid (mRNA), such as a pre-messenger ribonucleic acid (pre-mRNA)) at a target sequence (such as described herein), and (ii) a recruiting moiety (such as described herein) configured to recruit a post-transcriptional regulating moiety (e.g., a spliceosomal moiety) (such as described herein), wherein the set of engineered polynucleotides are configured to specifically bind the RNA (e.g., the mRNA, such as the pre-mRNA) at a plurality of target sequences comprising the target sequence (such as described herein).

### Vectors

In some embodiments described herein include a vector or a plasmid comprising a nucleic acid sequence encoding an engineered polynucleotide as described herein.

In some embodiments described herein include a plurality of vectors or a plurality of plasmids comprising a plurality of nucleic acid sequences each encoding an engineered polynucleotide as described herein. In some embodiments, the plurality of vectors or the plurality of plasmids comprise the plurality of nucleic acid sequences encoding more than one engineered polynucleotide as described herein. In some embodiments, the plurality of vectors or the plurality of plasmids comprise the plurality of nucleic acid sequences encoding a plurality of engineered polynucleotides (each independently described herein).

### Pharmaceutical Composition(s)

Described herein, in some embodiments, is a pharmaceutical composition comprising the engineered polynucleotide described herein, or a plasmid, a vector, or an isolated DNA encoding the sequence thereof. Pharmaceutical composition, as used herein, refers to a mixture of at least one engineered polynucleotide or a vector encoding the at least one engineered polynucleotide, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the engineered polynucleotide, the compositions, the cells contacted with the engineered polynucleotide or contacted with the composition comprising the engineered polynucleotide, or a combination thereof.

The pharmaceutical formulations described herein are administered to a subject by appropriate administration routes, including but not limited to, intravenous, intrathecal, intraarterial, intratumoral, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

### Kit(s)

Described herein, in some embodiments, are kits for using the engineered polynucleotide, the compositions, or the pharmaceutical compositions described herein. In some embodiments, the kits disclosed herein may be used to treat a disease or condition in a subject. In some embodiments, the kit comprises an assemblage of materials or components apart from the engineered polynucleotide, the composition, or the pharmaceutical composition. In some embodiments, the kit comprises the components for assaying and selecting for suitable oligonucleotide for treating a disease or a condition. In some embodiments, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the engineered polynucleotide. In some embodiments, the kit comprises instructions thawing or otherwise restoring biological activity of the engineered polynucleotide, which may have been cryopreserved or lyophilized during storage or transportation. In some embodiments, the kit comprises instructions for measuring efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the engineered polynucleotide, the composition, or the pharmaceutical composition may be in dissolved, dehydrated, or lyophilized form. The components are typically contained in suitable packaging material(s).

### Methods

Described herein are methods for utilizing an engineered polynucleotide (such as described herein), such as a method for altering a ribonucleic acid (RNA) (e.g., a messenger ribonucleic acid (mRNA), such as a pre-messenger ribonucleic acid (pre-mRNA)) in a cell. The method may comprise contacting the cell with the engineered polynucleotide (such as described herein) that comprises one or more targeting moiety and a recruiting moiety. The one or more targeting moiety may bind to the RNA (e.g., the mRNA, such as the pre-mRNA) (such as described herein) at a target sequence (such as described herein) therein, and the recruiting moiety recruits a post-transcriptional regulating moiety (e.g., a spliceosomal moiety) (such as described herein) within proximity of the target sequence of the RNA (e.g., the mRNA, such as the pre-mRNA) to alter the RNA (e.g., the mRNA, such as the pre-mRNA) in the cell, thereby yielding one or more altered RNA (e.g., one or more altered mRNA, such as one or more altered pre-mRNA). In some embodiments, the method alters an expression or activity of the target gene. In some embodiments, prior to the contacting, the cell exhibits an aberrant messenger ribonucleic acid (mRNA) or protein corresponding to the target gene. In some embodiments, a targeting moiety of the one or more targeting moiety is sufficiently identical or complementary to a consensus sequence in the target sequence of a target gene (e.g., microtubule associated protein TAU (MAPT)).

Described herein include a method for altering a ribonucleic acid (RNA) (e.g., a messenger ribonucleic acid (mRNA), such as a pre-messenger ribonucleic acid (pre-mRNA)) at a plurality of locations in a cell. The method may comprise contacting the cell with a set of engineered polynucleotides (such as each independently described herein). An engineered polynucleotide may comprise one or more targeting moiety and a recruiting moiety. The one or more targeting moiety may bind to the RNA (e.g., the mRNA, such as the pre-mRNA) (such as described herein) at a plurality of target sequences (such as described herein) therein. Each recruiting moiety may recruit a post-transcriptional regulating moiety (e.g., a spliceosomal moiety) (such as described herein) within proximity of a target sequence of the RNA (e.g., the mRNA, such as the pre-mRNA) to alter the RNA (e.g., the mRNA, such as the pre-mRNA) in the cell, thereby yielding one or more altered RNA (e.g., one or more altered mRNA, such as one or more altered pre-mRNA). In some embodiments, the method alters an expression or activity of the target gene by altering (e.g., cleaving or/and chemically modifying) the RNA (e.g., the mRNA, such as the pre-mRNA) at the plurality of locations. In some embodiments, prior to the contacting, the cell exhibits an aberrant messenger ribonucleic acid (mRNA) or protein corresponding to the target gene. In some embodiments, one or each targeting moiety of the one or more targeting moiety is sufficiently identical or complementary to a consensus sequence in the target sequence of a target gene (e.g., microtubule associated protein TAU (MAPT)).

In some embodiments, the method comprises delivering the engineered polynucleotide into a cell. In some embodiments, the method comprises delivering a polynucleotide encoding the engineered polynucleotide into a cell and subsequently expressing the engineered polynucleotide for modulating the expression or the activity of a gene encoded by the target sequence described herein. In some embodiments, the method comprises using the engineered polynucleotide for treating a disease or condition in a subject in need thereof. The disease or condition may be associated with an aberrant expression or activity of a target gene encoded by the RNA (e.g., the mRNA, such as the pre-mRNA). In some embodiments, the RNA (e.g., the mRNA, such as the pre-mRNA) corresponds to a target gene (e.g., microtubule associated protein TAU (MAPT)).

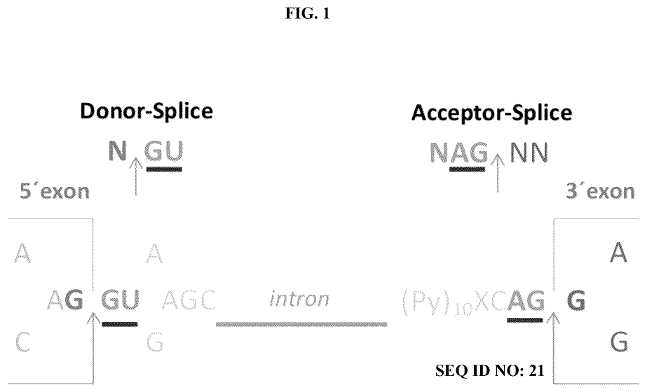

FIG. 1 illustrates a schematic diagram for identifying splice donor and acceptor for designing the nucleotide sequence of the engineered polynucleotide, where the engineered polynucleotides or the methods described herein present an improvement over the currently available approaches for modulating expression or activity of a gene for treating a disease or condition. In some embodiments, the methods described herein modulate the expression or activity of a gene by the engineered polynucleotide targeting the transcript of the gene that causes a disease or condition. In some embodiments, the methods described herein comprise administering the engineered polynucleotide described herein to a subject in need thereof. In some cases, the methods described herein comprise utilizing the engineered polynucleotide to recruit a regulating moiety to modulate the expression or activity of the gene that causes the disease or condition, thereby treating the disease or condition. In some aspects, the methods described herein comprise utilizing the engineered polynucleotide to stabilize the assembly of the regulating moiety to modulate the expression or activity of the gene that causes the disease or condition, thereby treating the disease or condition.

Described herein, in some embodiments, are methods of delivering the engineered polynucleotides described herein to a cell. In some embodiments, the method comprises delivering directly or indirectly engineered polynucleotides to the cell. In some embodiments, the method comprises contacting the cell with a composition comprising the engineered polynucleotide described herein. In some embodiments, the method comprises expressing the engineered polynucleotide described herein in the cell. In some embodiments, the engineered polynucleotide or vector encoding the engineered polynucleotide can be delivered into the cell via any of the transfection methods described herein. In some embodiments, the engineered polynucleotide can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the engineered polynucleotide or vector encoding the engineered polynucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of engineered polynucleotide or vector encoding the engineered polynucleotide into a host cell is calcium phosphate transfection.

Chemical means for introducing the engineered polynucleotide or vector encoding the engineered polynucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of engineered polynucleotide or vector encoding the engineered polynucleotide with targeted nanoparticles.

In the case where a non-viral delivery system is utilized, an example delivery vehicle is a liposome. The use of lipid formulations is contemplated for the introduction of the engineered polynucleotide or vector encoding the engineered polynucleotide into a cell (in vitro, ex vivo, or in vivo). In another aspect, the engineered polynucleotide or vector encoding the engineered polynucleotide can be associated with a lipid. The engineered polynucleotide or vector encoding the engineered polynucleotide associated with a lipid can be encapsulated in the aqueous interior of a liposome, interspersed within the lipid bilayer of a liposome, attached to a liposome via a linking molecule that is associated with both the liposome and the engineered polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the engineered polynucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the engineered polynucleotide or vector encoding the engineered polynucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

Described herein, in some aspects, are methods for modulating or altering expression or activity of a gene encoded by a target sequence in a cell. In some embodiments, the target sequence is a pre-messenger ribonucleic acid (pre-mRNA) in a cell. In some embodiments, the method comprises contacting the cell with an engineered polynucleotide comprising one or more targeting moieties and a recruiting moiety. In some embodiments, the one or more targeting moieties bind to the pre-mRNA at a target sequence therein. In some embodiments, the recruiting moiety recruits a post-transcriptional regulating moiety (e.g., a spliceosomal moiety) within proximity of the target sequence of the pre-mRNA to alter the pre-mRNA in the cell, thereby yielding one or more altered pre-mRNA. In some embodiments, the pre-mRNA corresponds to a target gene such as microtubule associated protein TAU (MAPT). In some embodiments, the method, when the engineered polynucleotide binds and recruits the spliceosomal moiety to the target sequence, increases an expression or activity of the target gene. In some embodiments, the method, when the engineered polynucleotide binds and recruits the spliceosomal moiety to the target sequence, decreases an expression or activity of the target gene. In some embodiments, the method, when the engineered polynucleotide binds and recruits the spliceosomal moiety to the target sequence, corrects aberrant messenger ribonucleic acid (mRNA) or protein corresponding to the target gene.

In some embodiments, the method comprises contacting or delivering two or more engineered polynucleotides into a single cell, where the engineered polynucleotides each comprise one or more targeting moieties configured to bind to two or more target sequences. The two or more target sequences can be located on the same strand of the pre-mRNA encoding a target gene. The two or more target sequences can be located on different strands of pre-mRNA encoding the same target gene. The two or more target sequences can be located on different strands of pre-mRNA, where each strand of the pre-mRNA can encode different target gene. In some embodiments, the method comprises two or more engineered polynucleotides configured to specifically bind the pre-mRNA at a plurality of target sequences comprising the target sequence.

Disclosed herein, in some embodiments, are methods of treating a disease or condition by modulating expression or activity of a target gene in a cell, thereby treating the disease or condition. In some embodiments, the method comprises treating a disease or condition by correcting aberrant messenger ribonucleic acid (mRNA) or protein corresponding to the target gene. In some embodiments, the disease or condition is associated with increased expression or activity of any one of the target gene described herein. In some embodiments, the disease or condition is associated with decreased expression or activity of any one of the target gene described herein. In some embodiments, the disease or condition is associated with splicing of aberrant messenger ribonucleic acid (mRNA) or protein corresponding to of any one of the target gene described herein.

In some embodiments, the engineered polynucleotide or pharmaceutical composition comprising the engineered polynucleotide can be administered to the subject alone (e.g., standalone treatment). In some embodiments, the engineered polynucleotide or pharmaceutical composition comprising the engineered polynucleotide is administered in combination with an additional agent. In some cases, the additional agent as used herein is administered alone. The engineered polynucleotide or pharmaceutical composition comprising the engineered polynucleotide and the additional agent can be administered together or sequentially. The combination therapies can be administered within the same day, or can be administered one or more days, weeks, months, or years apart.

In some embodiments, the engineered polynucleotide or pharmaceutical composition comprising the engineered polynucleotide is administered at a concentration of at least 0.25 μM. In some embodiments, the engineered polynucleotide or pharmaceutical composition comprising the engineered polynucleotide is administered at a concentration of at least 0.5 μM. In some embodiments, the engineered polynucleotide or pharmaceutical composition comprising the engineered polynucleotide is administered at a concentration of at least 0.75 μM. In some embodiments, the engineered polynucleotide or pharmaceutical composition comprising the engineered polynucleotide is administered at a concentration of at least 1 μM. In some embodiments, the engineered polynucleotide or pharmaceutical composition comprising the engineered polynucleotide is administered at a concentration of at least 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, or more. In some embodiments, the engineered polynucleotide or pharmaceutical composition comprising the engineered polynucleotide is administered at a concentration of no more 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, or less. In some embodiments, the engineered polynucleotide or pharmaceutical composition comprising the engineered polynucleotide is administered at a concentration of about 0.25 μM to 1 μM.

In some embodiments, the engineered polynucleotide or pharmaceutical composition comprising the engineered polynucleotide is a first-line treatment for the disease or condition. In some embodiments, the engineered polynucleotide or pharmaceutical composition comprising the engineered polynucleotide is a second-line, third-line, or fourth-line treatment. In some embodiments, the engineered polynucleotide or pharmaceutical composition comprising the engineered polynucleotide comprises at least one, two, three, four, five, six, seven, eight, nine, 10, 20, 30 or more oligonucleotide. In general, method disclosed herein comprises administering the engineered polynucleotide or pharmaceutical composition comprising the engineered polynucleotide by oral administration. However, in some instances, method comprises administering the engineered polynucleotide or pharmaceutical composition comprising the engineered polynucleotide by intraperitoneal injection. In some instances, the method comprises administering the engineered polynucleotide or pharmaceutical composition comprising the engineered polynucleotide by intravenous (“i.v.”) administration. In some instances, the method comprises administering the engineered polynucleotide or pharmaceutical composition comprising the engineered polynucleotide by intratumoral administration. It is conceivable that one can also administer the engineered polynucleotide or pharmaceutical composition comprising the engineered polynucleotide disclosed herein by other routes such as subcutaneous injection, intramuscular injection, intradermal injection, transdermal injection percutaneous administration, intranasal administration, intralymphatic injection, intrathecal administration, pulmonary administration, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

Suitable dose and dosage administrated to a subject is determined by factors including, but no limited to, the particular the engineered polynucleotide, composition, or pharmaceutical composition, disease condition and its severity, comorbidities, the identity (e.g., weight, sex, age) of the subject in need of treatment, and can be determined according to the particular circumstances surrounding the case, including, e.g., the specific agent being administered, the route of administration, the condition being treated, and the subject being treated.

Described herein are methods of treating cancer in a subject in need thereof using the engineered polynucleotide or vector encoding the engineered polynucleotide. The engineered polynucleotide can be a engineered polynucleotide as described in PCT/US2022/037391, of which is incorporated by reference in its entirety.

Described herein are methods of treating cancer in a subject in need thereof comprising administering to the subject a pharmaceutical composition comprising an engineered polynucleotide. In some embodiments, the engineered polynucleotide comprises: (i) one or more targeting moieties configured to specifically bind a pre-messenger ribonucleic acid (pre-mRNA) at a target sequence therein (e.g., a conserved splice site); and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

In some embodiments, the cancer is selected from the group consisting of: brain cancer, prostate cancer, breast cancer, kidney cancer, lung cancer, and liver cancer. In some embodiments, the cancer is a glioblastoma. In some embodiments, the cancer is a breast adenocarcinoma. In some embodiments, the cancer is a neuroblastoma. In some embodiments, the cancer is a prostate adenocarcinoma. In some embodiments, the cancer is a renal carcinoma. In some embodiments, the cancer is a hepatocellular carcinoma. In some embodiments, the cancer is a kidney adenocarcinoma.

In some embodiments, provided herein is a method, comprising: contacting a cell with an engineered polynucleotide or vector encoding the engineered polynucleotide as described herein. In some embodiments, contacting a cell with the engineered polynucleotide or vector encoding the engineered polynucleotide results in a cytotoxic or cytostatic effect. In some embodiments, contacting a cell with the engineered polynucleotide or vector encoding the engineered polynucleotide results in altered cell viability. In some embodiments, contacting a cell with the engineered polynucleotide or vector encoding the engineered polynucleotide results in internalization of the engineered polynucleotide by the cell. In some embodiments, contacting a cell with the engineered polynucleotide or vector encoding the engineered polynucleotide results in reduced mitochondrial activity. In some embodiments, contacting a cell with the engineered polynucleotide or vector encoding the engineered polynucleotide results in reduced cell proliferation. In some embodiments, contacting a cell with the engineered polynucleotide or vector encoding the engineered polynucleotide results in a change in the distribution of cells cycle phases wherein cell cycle phases consist of: Sub/G1: non-proliferative state (quiescence); G1/G0: cell growth; S: DNA replication; G2/M: DNA segregation and mitosis. In some embodiments, contacting a cell with the engineered polynucleotide or vector encoding the engineered polynucleotide results in an increase in the propensity of a cell to be in the G2/M phase.

In some embodiments, contacting a cell with the engineered polynucleotide or vector encoding the engineered polynucleotide results in an increase in apoptosis or necrosis. In some embodiments, contacting a cell with the engineered polynucleotide or vector encoding the engineered polynucleotide results in a reduction in MAPT expression. In some embodiments, contacting a cell with the engineered polynucleotide or vector encoding the engineered polynucleotide results in a reduction in the quantity of TAU. In some embodiments, contacting a cell with the engineered polynucleotide or vector encoding the engineered polynucleotide results in a change in mitochondrial membrane potential.

In some embodiments, provided herein is a method, comprising: contacting a plurality of cells with an engineered polynucleotide or vector encoding the engineered polynucleotide as described herein. In some embodiments, contacting the plurality of cells with the engineered polynucleotide or vector encoding the engineered polynucleotide results in a change in the distribution of cells cycle phases wherein cell cycle phases consist of: Sub/G1: non-proliferative state (quiescence); G1/G0: cell growth; S: DNA replication; G2/M: DNA segregation and mitosis. In some embodiments, contacting the plurality of cells with the engineered polynucleotide or vector encoding the engineered polynucleotide results in an increase in number of cells in the G2/M phase. In some embodiments, contacting a cell with the engineered polynucleotide or vector encoding the engineered polynucleotide results in an increase in number of cells in a necrotic or apoptotic phase.

In some embodiments, provided herein is a method for treating a subject having or suspected of having cancer. In some embodiments, provided herein is a method for treating a tumor in a subject. In some embodiments, contacting a tumor in a subject with the engineered polynucleotide or vector encoding the engineered polynucleotide results in a reduction in tumor progression. In some embodiments, the tumor is a glioblastoma. In some embodiments, the engineered polynucleotide or vector encoding the engineered polynucleotide is administered intravenously. In some embodiments, the engineered polynucleotide or vector encoding the engineered polynucleotide is administered intratumorally. In some embodiments, the engineered polynucleotide or vector encoding the engineered polynucleotide is administered subcutaneously. In some embodiments, the engineered polynucleotide or vector encoding the engineered polynucleotide is administered once. In some embodiments, the engineered polynucleotide or vector encoding the engineered polynucleotide is administered daily. In some embodiments, the engineered polynucleotide or vector encoding the engineered polynucleotide is administered daily for between 1 and 21 days. In some embodiments, administration of the engineered polynucleotide or vector encoding the engineered polynucleotide to a subject in need thereof results in reduced tumor volume. In some embodiments, administration of the engineered polynucleotide or vector encoding the engineered polynucleotide to a subject in need thereof results in reduced tumor volume ratio.

In some embodiments, the cell is a glioblastoma. In some embodiments, the cell is a breast adenocarcinoma. In some embodiments, the cell is a neuroblastoma. In some embodiments, the cell is a prostate adenocarcinoma. In some embodiments, the cell is a renal carcinoma. In some embodiments, the cell is a hepatocellular carcinoma. In some embodiments, the cell is a kidney adenocarcinoma. In embodiments, the cell is a neuron. In some embodiments, the cell is from a subject diagnosed with cancer. In some embodiments, the cell is a neuron from a subject diagnosed with cancer.

In some embodiments, the engineered polynucleotide is ASMO1. In some embodiments, ASMO1 interacts directly or indirectly with U1-C and U1-70K. In some embodiments, ASMO1 interacts directly or indirectly with the U1 complex and with MAPT pre-mRNA.

In various embodiments, the subject is administered, co-administered, as has previously been administered a taxane. The taxane can comprise a taxane that has efficacy at treating cancer. Upon administration of the engineered polynucleotide, the prognosis of the subject may improve, for example, as compared to before the engineered polynucleotide was administered, or as compared to another subject that was administered a taxane and was not administered the engineered polypeptide. The engineered polypeptide may result in an altered amount of TAU present in the subject. Taxanes may be able to bind TAU, resulting in decrease of taxane able to bind to other targets in the cell. By decreasing the amount of TAU, the taxanes may have less “off-target” binding and may increase if efficacy. In various embodiments, the subject is not administered, co-administered, as has previously been administered a taxane.

In various embodiments, the subject administered the engineered polynucleotide without another anti-cancer drug or cancer therapeutic.

In various embodiments, the subject is administered, co-administered, as has previously been administered, another therapy for cancer (e.g., a drug, a neoadjuvant, a adjuvant). The administration of the engineered polynucleotide may improve the prognosis of the subject, or improve the treatment efficacy, as compared to administration with other therapy alone.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as example(s).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### LIST OF EMBODIMENTS

Embodiment 1. An engineered polynucleotide comprising: one or more targeting moieties configured to specifically bind a pre-messenger ribonucleic acid (pre-mRNA) at a target sequence therein; and a recruiting moiety configured to recruit a post-transcriptional regulating moiety (e.g., a spliceosomal moiety), wherein, when associated with said pre-mRNA and said engineered polynucleotide, said post-transcriptional regulating moiety alters said pre-mRNA in or in proximity to said target sequence.

Embodiment 2. The engineered polynucleotide of embodiment 1, wherein a targeting moiety of said one or more targeting moieties is sufficiently identical or complementary to a consensus sequence in said target sequence of a target gene.

Embodiment 3. The engineered polynucleotide of embodiment 1 or 2, wherein said one or more targeting moieties comprise (1) a first targeting moiety configured to specifically bind a first targeted sequence in said target sequence of said pre-mRNA, and (2) a second targeting moiety configured to specifically bind a second targeted sequence in said target sequence of said pre-mRNA.

Embodiment 4. The engineered polynucleotide of embodiment 3, wherein said first targeted sequence comprises a consensus sequence in said target sequence.

Embodiment 5. The engineered polynucleotide of embodiment 3 or 4, wherein said second targeted sequence comprises a consensus sequence in said target sequence.

Embodiment 6. The engineered polynucleotide of any one of embodiments 3 to 5, wherein said first and second targeted sequences are apart in said target sequence by a spacing sequence of no more than five nucleotides (e.g., one or two nucleotides).

Embodiment 7. The engineered polynucleotide of any one of embodiments 1 to 6, wherein said target sequence comprises an exon-intron boundary in said pre-mRNA.

Embodiment 8. The engineered polynucleotide of embodiment 7, wherein said first and second targeted sequences are both 5′ or 3′ with respect to said exon-intron boundary.

Embodiment 9. The engineered polynucleotide of embodiment 7, wherein one of said first and second targeted sequences is 5′ with respect to said exon-intron boundary; and wherein the other of said first and second targeted sequences is 3′ with respect to said exon-intron boundary.

Embodiment 10. The engineered polynucleotide of any one of embodiments 1 to 9, wherein said target sequence comprises a splice site in said pre-mRNA.

Embodiment 11. The engineered polynucleotide of embodiment 10, wherein said first or second targeted sequence comprises a splice site (e.g., 5′ ss) in said pre-mRNA.

Embodiment 12. The engineered polynucleotide of any one of embodiments 1 to 11, wherein one of said first and second targeting moieties is 5′ with respect to said recruiting moiety, and the other of said first and second targeting moieties is 3′ with respect to said recruiting moiety

Embodiment 13. The engineered polynucleotide of embodiment 12, wherein said first targeting moiety or said second targeting moiety comprises a sequence identical or complementary to a sequence set forth in Table 1.

Embodiment 14. The engineered polynucleotide of embodiment 12, wherein said first targeting moiety comprises a sequence identical or complementary to a sequence selected from the exon sequence column of Table 1; and wherein said second targeting moiety comprises a sequence identical or complementary to a sequence set forth in the intron sequence column of Table 1.

Embodiment 15. The engineered polynucleotide of embodiment 12, wherein said first targeting moiety comprises a sequence identical or complementary to a sequence set forth in the intron sequence column of Table 1; and wherein said second targeting moiety comprises a sequence identical or complementary to a sequence set forth in the exon sequence column of Table 1.

Embodiment 16. The engineered polynucleotide of embodiment 12, wherein said first targeting moiety or said second targeting moiety comprises a sequence identical or complementary to a consensus sequence of an intron donor site (e.g., selected from GU, GT, GC, and CA).

Embodiment 17. The engineered polynucleotide of embodiment 12, wherein said first targeting moiety or said second targeting moiety comprises a sequence identical or complementary to a consensus sequence of an exon donor site (e.g., G).

Embodiment 18. The engineered polynucleotide of embodiment 12, wherein said first targeting moiety or said second targeting moiety comprises a sequence identical or complementary to a consensus sequence selected from GU, GC, G, and CA.

Embodiment 19. The engineered polynucleotide of any one of embodiments 1 to 18, wherein said spliceosomal moiety is selected from a spliceosomal ribonucleoprotein complex, a spliceosomal small nuclear ribonucleic acid (snRNA), a spliceosomal protein, a functional variant thereof, or a functional fragment thereof.

Embodiment 20. The engineered polynucleotide of embodiment 19, wherein said spliceosomal moiety comprises U1 snRNA and a spliceosomal protein.

Embodiment 21. The engineered polynucleotide of any one of embodiments 19 to 20, wherein said spliceosomal snRNA is selected from U1, U2, U4, U5, U6, U11, U12, U14atac, U6atac, and combinations thereof.

Embodiment 22. The engineered polynucleotide of any one of embodiments 19 to 21, wherein said spliceosomal protein is selected from Sm, U1-70k, U1A, U1C, and combinations thereof.

Embodiment 23. The engineered polynucleotide of any one of embodiments 1 to 22, wherein said recruiting moiety comprises a nucleotide sequence that is at least 70%, 80%, 85%, or 90% identical or complementary to any one of SEQ ID NOs: 1-2.

Embodiment 24. The engineered polynucleotide of embodiment 23, wherein said recruiting moiety comprises a nucleotide sequence that is identical or complementary to any one of SEQ ID NOs: 1-2.

Embodiment 25. The engineered polynucleotide of any one of embodiments 1 to 24, wherein said engineered polynucleotide comprises a (e.g., secondary) structural feature.

Embodiment 26. The engineered polynucleotide of embodiment 25, wherein said engineered polynucleotide comprises an apical loop, an upper stem, an internal loop, a lower stem, or a combination thereof

Embodiment 27. The engineered polynucleotide of embodiment 25, wherein said engineered polynucleotide comprises a loop (e.g., an internal loop) adjacent to a stem (e.g., a lower stem or an upper stem) comprising two complementary stem sequences.

Embodiment 28. The engineered polynucleotide of embodiment 27, wherein a stem sequence of said stem (e.g., said lower stem or said upper stem) comprises no more than about five, four, or three nucleotides.

Embodiment 29. The engineered polynucleotide of embodiment 27 or 28, wherein said loop is an internal loop adjacent to said stem (e.g., said lower stem) and a further stem (e.g., an upper stem) comprising two complementary stem sequences.

Embodiment 30. The engineered polynucleotide of embodiment 29, wherein said internal loop comprises a nucleic acid sequence of no more than 10, 9, or 8 nucleotides.

Embodiment 31. The engineered polynucleotide of embodiment 29 or 30, wherein a stem sequence of said further stem (e.g., said upper stem) comprises no more than about five, four, or three nucleotides.

Embodiment 32. The engineered polynucleotide of any one of embodiments 29 to 31, wherein said engineered polynucleotide further comprises an apical loop.

Embodiment 33. The engineered polynucleotide of embodiment 32, wherein said apical loop comprises a nucleic acid sequence of no more than 10, 9, 8, 7, 6, or 5 nucleotides.

Embodiment 34. The engineered polynucleotide of any one of embodiments 1 to 33, wherein said engineered polynucleotide does not comprise any intramolecular disulfide bond.

Embodiment 35. The engineered polynucleotide of any one of embodiments 1 to 34, wherein, when associated with said engineered polynucleotide and said spliceosomal moiety, said pre-mRNA exhibits substantially no base pairing with an RNA binding domain (RBD) of U1 snRNA.

Embodiment 36. The engineered polynucleotide of any one of embodiments 1 to 35, wherein, when associated with said engineered polynucleotide and said spliceosomal moiety, said pre-mRNA exhibits substantially no base-specific interaction with U1-C protein.

Embodiment 37. The engineered polynucleotide of any one of embodiments 1 to 36, wherein said engineered polynucleotide is configured to specifically interact with zinc-finger of U1-C protein.

Embodiment 38. The engineered polynucleotide of embodiment 37, wherein a 5′-targeting moiety of said engineered polynucleotide is configured to specifically interact with zinc-finger of U1-C protein.

Embodiment 39. The engineered polynucleotide of embodiment 37 or 38, wherein (e.g., said 5′-targeting moiety of) said engineered polynucleotide is configured to covalently interact (e.g., via disulfide bonding) with zinc-finger of U1-C protein.

Embodiment 40. The engineered polynucleotide of any one of embodiments 37 to 39, wherein (e.g., said 5′-targeting moiety of) said engineered polynucleotide is configured to non-covalently interact (e.g., via hydrogen bonding) with zinc-finger of U1-C protein.

Embodiment 41. The engineered polynucleotide of any one of embodiments 1 to 40, wherein said engineered polynucleotide comprises a nucleotide sequence complementary to a partial sequence of Stem-Loop II (SL2) of U1 snRNA.

Embodiment 42. The engineered polynucleotide of embodiment 41, wherein a side of a stem-loop structure of said engineered polynucleotide comprises a nucleotide sequence complementary to a partial sequence of Stem-Loop II (SL2) of U1 snRNA.

Embodiment 43. The engineered polynucleotide of embodiment 41 or 42, wherein said partial sequence comprises the sequence corresponding to 5′-GGCCU-3′ of SL2 of U1 snRNA.

Embodiment 44. The engineered polynucleotide of any one of embodiments 41 to 43, wherein said partial sequence does not comprise the sequence corresponding to 5′-CACGUUA-3′ of SL2 of U1 snRNA.

Embodiment 45. The engineered polynucleotide of any one of embodiments 1 to 44, wherein said engineered polynucleotide exhibits substantially no base pairing with an anchoring sequence of SL2 of U1 snRNA.

Embodiment 46. The engineered polynucleotide of embodiment 45, wherein an internal loop of said engineered polynucleotide exhibits substantially no base pairing with said anchoring sequence of said SL2 of U1 snRNA.

Embodiment 47. The engineered polynucleotide of embodiment 45 or 46, wherein a lower stem of said engineered polynucleotide exhibits substantially no base pairing with said anchoring sequence of said SL2 of U1 snRNA.

Embodiment 48. The engineered polynucleotide of any one of embodiments 45 to 47, wherein said anchoring sequence comprises the sequence corresponding to 5′-CACGUUA-3′.

Embodiment 49. The engineered polynucleotide of any one of embodiments 1 to 48, wherein said engineered polynucleotide exhibits substantially no base pairing with H helix of U1 snRNA.

Embodiment 50. The engineered polynucleotide of any one of embodiments 1 to 49, wherein said engineered polynucleotide comprises at least one chemical modification.

Embodiment 51. The engineered polynucleotide of embodiment 50, wherein said engineered polynucleotide comprises at least one 2′-modified (e.g., 2′-methoxy, 2′-methoxymethyl, or 2′-methoxyethyl) nucleotides.

Embodiment 52. The engineered polynucleotide of embodiment 50 or 51, wherein at least about 50%, 60%, 70%, 80%, or 90% nucleotides of said engineered polynucleotide are chemically modified nucleotides.

Embodiment 53. The engineered polynucleotide of embodiment 50 or 51, wherein at least about 50%, 60%, 70%, 80%, or 90% nucleotides of said engineered polynucleotide are 2′-modified (e.g., 2′-methoxy, 2′-methoxymethyl, or 2′-methoxyethyl) nucleotides.

Embodiment 54. The engineered polynucleotide of any one of embodiments 50 to 53, wherein said engineered polynucleotide comprises at least one phosphorothioate internucleotide bond.

Embodiment 55. The engineered polynucleotide of any one of embodiments 50 to 54, wherein at least about 50%, 60%, 70%, 80%, or 90% internucleotide linkages of said engineered polynucleotide are chemically modified.

Embodiment 56. The engineered polynucleotide of any one of embodiments 50 to 54, wherein at least about 50%, 60%, 70%, 80%, or 90% internucleotide linkages are phosphorathioate.

Embodiment 57. The engineered polynucleotide of any one of embodiments 1 to 56, wherein said engineered polynucleotide comprises about 10 to about 40 nucleotides, about 10 to about 35 nucleotides, about 10 to about 30 nucleotides, or about 10 to about 25 nucleotides.

Embodiment 58. The engineered polynucleotide of any one of embodiments 1 to 57, wherein said recruiting moiety comprises about 10 to about 30 nucleotides, or about 10 to about 20 nucleotides.

Embodiment 59. The engineered polynucleotide of any one of embodiments 1 to 58, wherein said one or more targeting moieties each independently comprises about 2 to about 15 nucleotides, about 2 nucleotides to about 10 nucleotides, or about 2 nucleotides to about 8 nucleotides.

Embodiment 60. The engineered polynucleotide of any one of embodiments 1 to 59, wherein one of said first and second targeting moieties comprises about 2 nucleotides, and the other of said first and second targeting moieties comprises about 5 or 6 nucleotides.

Embodiment 61. The engineered polynucleotide of any one of embodiments 1 to 60, wherein, when associated with said engineered polynucleotide and said pre-mRNA, said spliceosomal moiety cleaves or splices said pre-mRNA in said target sequence.

Embodiment 62. The engineered polynucleotide of any one of embodiments 1 to 61, wherein said spliceosomal moiety further facilitates modification of a cleaved pre-mRNA.

Embodiment 63. An engineered polynucleotide comprising a nucleotide sequence that is at least 70%, 80%, 85%, or 90% identical or complementary to any one of SEQ ID NOs: 1-4, which engineered polynucleotide is characterized by a (e.g., secondary) structural feature.

Embodiment 64. The engineered polynucleotide of embodiment 63, wherein said nucleotide sequence is identical or complementary to any one of SEQ ID NOs: 1-4.

Embodiment 65. The engineered polynucleotide of embodiment 63 or 64, wherein said structural feature comprises one or more stem-loop structures.

Embodiment 66. The engineered polynucleotide of embodiment 63 or 64, wherein said structural feature comprises an apical loop, an upper stem, an internal loop, a lower stem, or a combination thereof.

Embodiment 67. The engineered polynucleotide of any one of embodiments 63 to 66, wherein said engineered polynucleotide comprises a loop (e.g., an internal loop) adjacent to a stem (e.g., a lower stem or an upper stem) comprising two complementary stem sequences.

Embodiment 68. The engineered polynucleotide of embodiment 67, wherein a stem sequence of said stem (e.g., said lower stem or said upper stem) comprises no more than about five, four, or three nucleotides.

Embodiment 69. The engineered polynucleotide of any one of embodiments 63 to 68, wherein said loop is an internal loop adjacent to said stem (e.g., said lower stem) and a further stem (e.g., an upper stem) comprising two complementary stem sequences.

Embodiment 70. The engineered polynucleotide of embodiment 69, wherein said internal loop comprises a nucleic acid sequence of no more than 10, 9, or 8 nucleotides.

Embodiment 71. The engineered polynucleotide of embodiment 69 or 70, wherein a stem sequence of said further stem (e.g., said upper stem) comprises no more than about five, four, or three nucleotides.

Embodiment 72. The engineered polynucleotide of any one of embodiments 69 to 71, wherein said engineered polynucleotide further comprises an apical loop.

Embodiment 73. The engineered polynucleotide of embodiment 72, wherein said apical loop comprises a nucleic acid sequence of no more than 10, 9, 8, 7, 6, or 5 nucleotides.

Embodiment 74. The engineered polynucleotide of any one of embodiments 63 to 73, wherein said engineered polynucleotide further comprises one or more targeting moieties sufficiently identical or complementary to a target sequence of a target gene.

Embodiment 75. The engineered polynucleotide of embodiment 74, wherein a targeting moiety of said one or more targeting moieties is sufficiently identical or complementary to a consensus sequence in said target sequence of said target gene.

Embodiment 76. The engineered polynucleotide of embodiment 74, wherein said target gene is microtubule associated protein TAU (MAPT).

Embodiment 77. The engineered polynucleotide of any one of embodiments 63 to 75, wherein said engineered polynucleotide comprises at least one chemical modification.

Embodiment 78. The engineered polynucleotide of embodiment 77, wherein said engineered polynucleotide comprises at least one phosphorothioate internucleotide bond.

Embodiment 79. The engineered polynucleotide of embodiment 77, wherein at least about 50%, 60%, 70%, 80%, or 90% internucleotide linkages of said engineered polynucleotide are chemically modified.

Embodiment 80. The engineered polynucleotide of embodiment 77, wherein at least about 50%, 60%, 70%, 80%, or 90% internucleotide linkages are phosphorathioate.

Embodiment 81. The engineered polynucleotide of any one of embodiments 77 to 80, wherein said engineered polynucleotide comprises at least one 2′-modified (e.g., 2′-methoxy, 2′-methoxymethyl, or 2′-methoxyethyl) nucleotides.

Embodiment 82. The engineered polynucleotide of any one of embodiments 77 to 80, wherein at least about 50%, 60%, 70%, 80%, or 90% nucleotides of said engineered polynucleotide are chemically modified nucleotides.

Embodiment 83. The engineered polynucleotide of any one of embodiments 77 to 80, wherein at least about 50%, 60%, 70%, 80%, or 90% nucleotides of said engineered polynucleotide are 2′-modified (e.g., 2′-methoxy, 2′-methoxymethyl, or 2′-methoxyethyl) nucleotides.

Embodiment 84. The engineered polynucleotide of any one of embodiments 64 to 84, wherein said engineered polynucleotide comprises about 10 to about 40 nucleotides, about 10 to about 35 nucleotides, about 10 to about 30 nucleotides, or about 10 to about 25 nucleotides.

Embodiment 85. A method for altering a pre-messenger ribonucleic acid (pre-mRNA) in a cell, the method comprising contacting said cell with an engineered polynucleotide that comprises one or more targeting moieties and a recruiting moiety, wherein said one or more targeting moieties bind to said pre-mRNA at a target sequence therein, and said recruiting moiety recruits a post-transcriptional regulating moiety (e.g., a spliceosomal moiety) within proximity of said target sequence of said pre-mRNA to alter said pre-mRNA in said cell, thereby yielding one or more altered pre-mRNA.

Embodiment 86. The method of embodiment 85, wherein a targeting moiety of said one or more targeting moieties is sufficiently identical or complementary to a consensus sequence in said target sequence of a target gene.

Embodiment 87. The method of embodiment 85 or 86, wherein said pre-mRNA corresponds to a target gene.

Embodiment 88. The method of embodiment 87, wherein said target gene is microtubule associated protein TAU (MAPT).

Embodiment 89. The method of any one of embodiments 85 to 88, wherein the method alters an expression or activity of said target gene.

Embodiment 90. The method of embodiment any one of embodiments 85 to 89, wherein, prior to said contacting, said cell exhibits an aberrant messenger ribonucleic acid (mRNA) or protein corresponding to said target gene.

Embodiment 91. A set of engineered polynucleotides each independently comprise: (i) one or more targeting moieties configured to bind a pre-messenger ribonucleic acid (pre-mRNA) at a target sequence, and (ii) a recruiting moiety configured to recruit a post-transcriptional regulating moiety (e.g., a spliceosomal moiety), wherein said set of engineered polynucleotides are configured to specifically bind said pre-mRNA at a plurality of target sequences comprising said target sequence.

### EXAMPLES

The following illustrative examples are representative of embodiments of the stimulation, systems, and methods described herein and are not meant to be limiting in any way.

### Example 1. Modulating Expression of Target Gene with Engineered Polynucleotide

Cells obtained from a cell like (e.g., HEK293 cell line) are cultured and maintained in cell culture medium. The cells can then be contacted with the engineered polynucleotide or a vector encoding the engineered polynucleotide for delivery of the engineered polynucleotide or a vector encoding the engineered polynucleotide into the cells by any one of the delivery method described herein. After the engineered polynucleotide is delivered into the cells, the cells can be cultured for a period of time to allow the engineered polynucleotide to modulate the expression or activity of the target gene. Cells can then be harvested and lysed for measurement of the expression or activity of the target gene. For example, the cells can be harvested and lysed and examined for the abundance of pre-mRNA, mRNA, or protein of the target gene modulated by the engineered polynucleotide. In other cases, the cells can be fixed and prepared for microscopic examination. For example, the cells can be examined under microscope for the presence or changes of the abundance of the inclusion body or amyloid plaque associated with any one of the target gene described herein (e.g., TAU plaques encoded by MAPT target gene).

### Example 2. In Vitro Evaluation of Cytotoxic Effects of ASMO1 (Also Known as “APT20TTMG” or “ASMO AP20TTMG”) on a Panel of Human Cancer Cell Lines

Cell lines were maintained in growth medium with 10% of Fetal Bovine Serum (FBS) until the time of the experiments. Cells were sub-cultured by trypsinization and splitting the cell suspension into fresh flasks and supplementing with fresh culture medium. To perform the assay, the cells were trypsinized, neutralized with complete medium (10% FBS), centrifuged, counted with assistance of a hemocytometer, and plated with the respective media with 10% FBS. Cell lines were plated at a density of 1×10⁴ cells per well, other than the SH-SY5Y cell line which was plated at a density of 2.5×10⁴. The preparation of the different experimental groups was done with 0.1% FBS (U87MG, MCF-7, SH-SY5Y and 786-O) and 1% FBS (PC-3). The untreated cells were used as negative control (medium only). After 24, 48, 72 and 96 hours of incubation, the effect of ASMO1 and controls on cytotoxicity of cells was determined by MTT assay.

Briefly, plates were taken out and 20 μl of 5 mg/ml of MTT 3-(4,5-dimethythiazol-2-yl)-2,5-diphenyl tetrazolium bromide solution was added to all the wells. Cells were incubated for 3 h at 37° C. After this period, supernatant was aspirated and 100 μl of DMSO was added to each well to dissolve formazan crystals. The absorbance of each well was then read at 540 nm using Synergy HT microplate reader.

GraphPad Prism 9 software (GraphPad Software Inc., San Diego, CA, USA) was used for graphics generation.

FIG. 11A-D demonstrate the effects of incubation with ASMO1 APT20TTMG in the U-87MG cell line. At 24 hours, it seems to exert a cytotoxic effect, since there is a trend of reduction in cell viability when compared to the negative control and vehicle groups. Within 48 hours, there is a reduction in absorbance values, which may indicate a cytotoxic and/or cytostatic effect. However, at times of 72 and 96 hours, the values are like those of the vehicle group, that is, the effect of reducing viability seems to have ended. Cells were plated and treated with 7 different concentrations (0.03, 0.1, 0.3, 1, 3, 10 and 30 μM) of APT20TTMG with medium (0.1% FBS). MTT assay was performed in 4 distinct times and graphs are represented as absorbance (540 nm). (A) 24 hours of incubation. (B) 48 hours of incubation. (C) 72 hours of incubation. (D) 96 hours of incubation.

As shown in FIG. 12A-D, in the MCF-7 cell line, it appears that ASMO1 APT20TTMG does not exert a cytotoxic or cytostatic effect. Cells were plated and treated with 7 different concentrations (0.03, 0.1, 0.3, 1, 3, 10 and 30 μM) of APT20TTMG with medium (0.1% FBS). MTT assay was performed in 4 distinct times and graphs are represented as absorbance (540 nm). (A) 24 hours of incubation. (B) 48 hours of incubation. (C) 72 hours of incubation. (D) 96 hours of incubation.

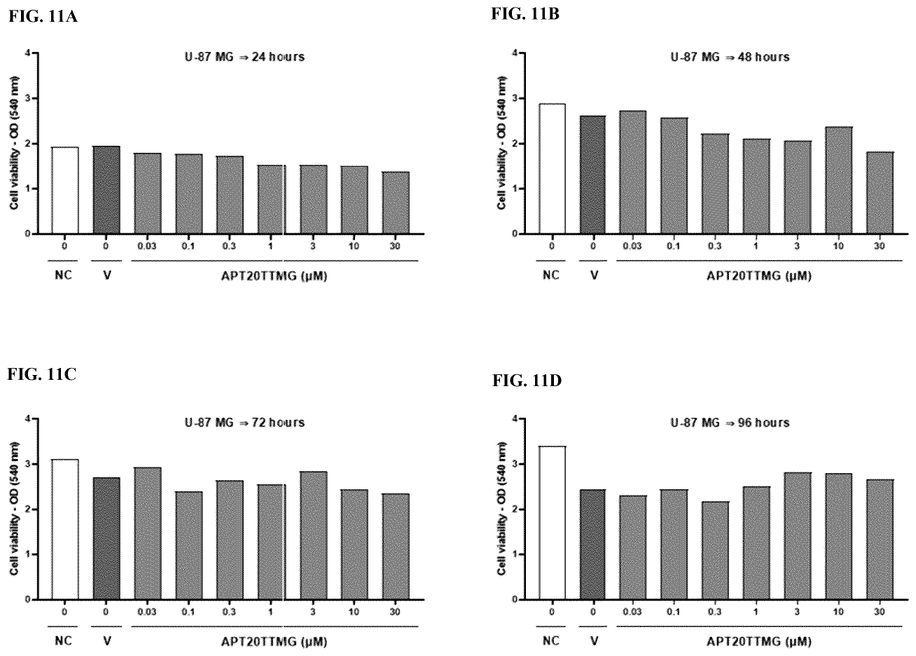

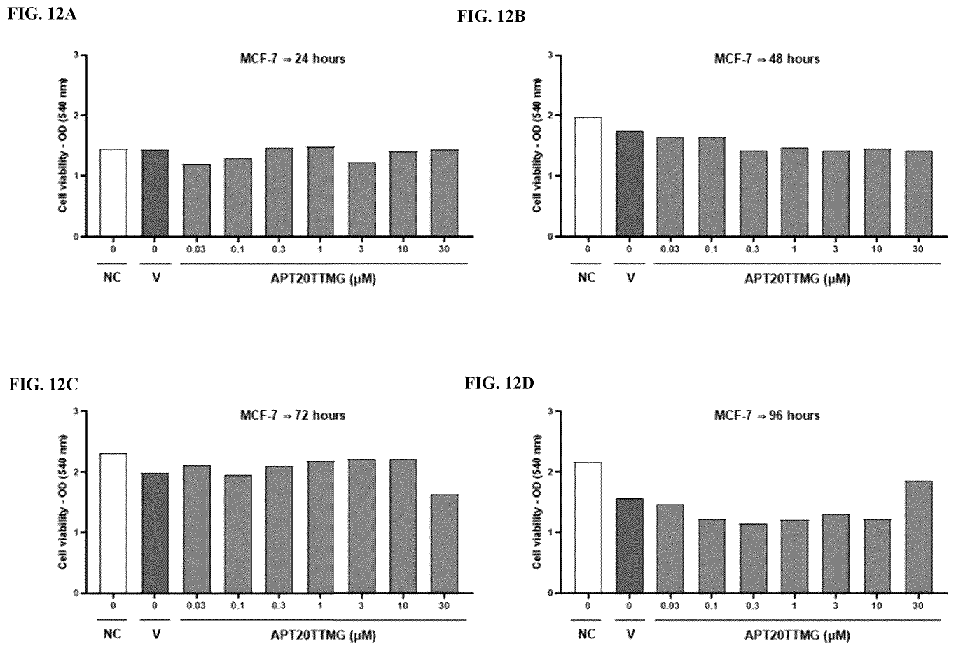

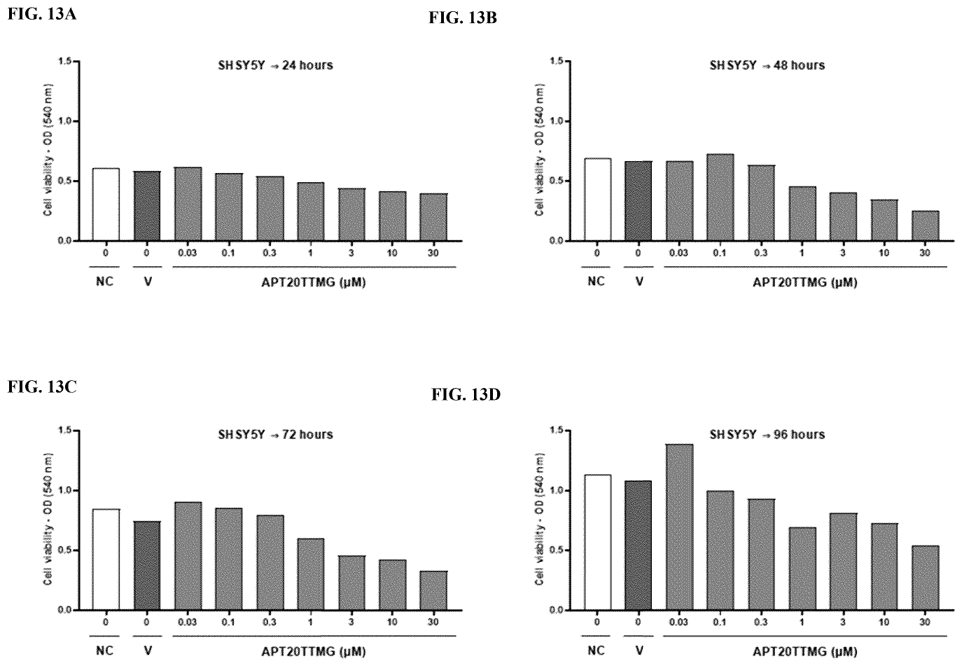

As shown in FIG. 13A-D, up to 72 hours ASMO1 APT20TTMG seems to have a cytotoxic effect from the concentration of 1p M. At 96 hours, the profile is a little different, and the concentration of 0.3 μM seems to start some effect. Cells were plated and treated with 7 different concentrations (0.03, 0.1, 0.3, 1, 3, 10 and 30 μM) of APT20TTMG with medium (0.1% FBS). MTT assay was performed in 4 distinct times and graphs are represented as absorbance (540 nm). (A) 24 hours of incubation. (B) 48 hours of incubation. (C) 72 hours of incubation. (D) 96 hours of incubation.

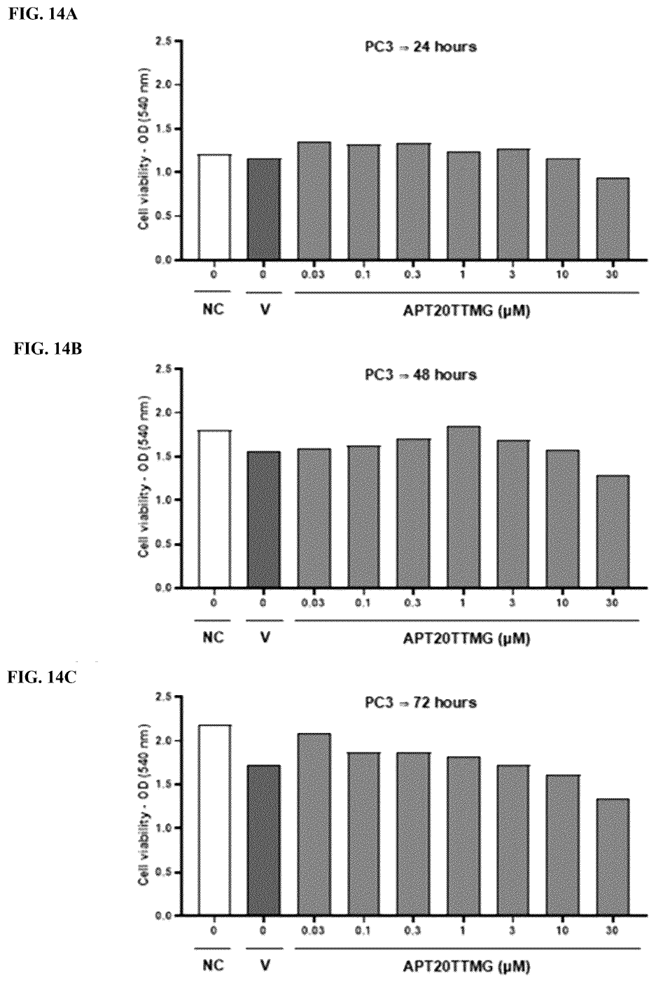

As shown in FIG. 14A-C, in the PC-3 cell line, it appears that ASMO1 APT20TTMG does not exert a cytotoxic or cytostatic effect. Cells were plated and treated with 7 different concentrations (0.03, 0.1, 0.3, 1, 3, 10 and 30 μM) of APT20TTMG with medium (1% FBS). MTT assay was performed in 3 distinct times and graphs are represented as absorbance (540 nm). (A) 24 hours of incubation. (B) 48 hours of incubation. (C) 72 hours of incubation.

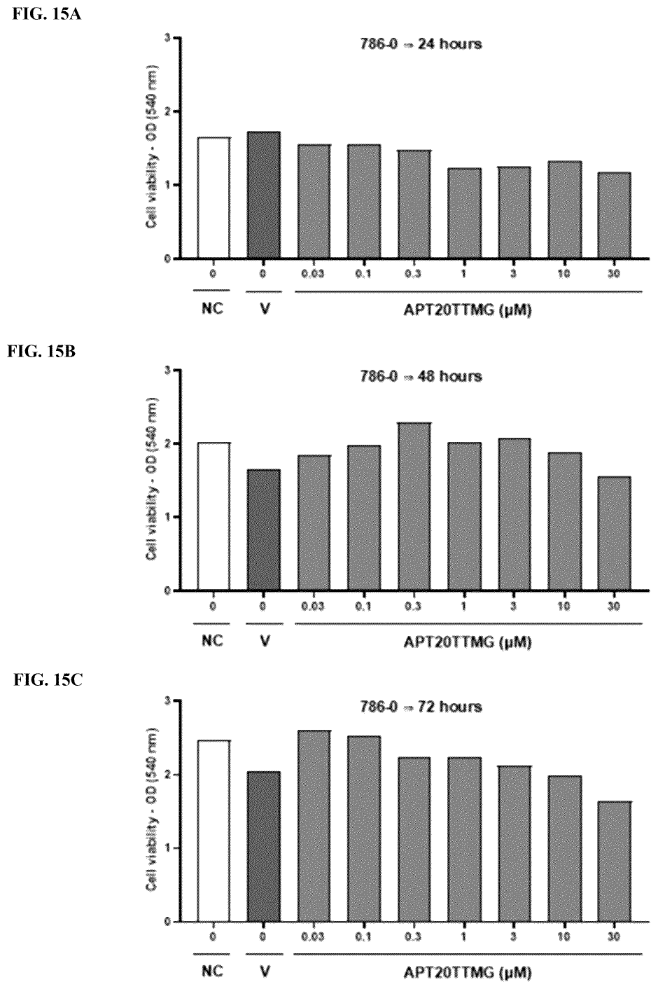

As shown in FIG. 15A-C, in 24 hours, it appears that ASMO1 APT20TTMG can alter cell viability in the 786-O line. However, in the other 2 times, there is no difference when compared to the control (vehicle group), only in the highest concentration (30 μM). Cells were plated and treated with 7 different concentrations (0.03, 0.1, 0.3, 1, 3, 10 and 30 μM) of APT20TTMG with medium (0.1% FBS). MTT assay was performed in 3 distinct times and graphs are represented as absorbance (540 nm). (A) 24 hours of incubation. (B) 48 hours of incubation. (C) 72 hours of incubation.

The results of this study demonstrated that the effects of ASMO1s are dependent on the type of cell line used, time and concentration, with improved efficacy in glioblastoma and neuroblastoma.

### Example 3. Evaluation of the Antitumor Potential of ASMO1 in Breast Carcinoma Cell Line (MCF-7)

MCF-7 cells were maintained in Roswell Park Memorial Institute 1640 medium (RPMI 1640) supplemented with 10% Fetal Bovine Serum (FBS) and 1% solution of Penicillin/Streptomycin. The cells were cultivated in cell culture flasks of 75 cm² and incubated at 37° C., 5% CO₂, and controlled humidity. After reaching 80-90% confluency, cells were washed with 10 mL of saline-buffered phosphate (PBS) and separated from the flasks with trypsin-EDTA solution (0.25%/0.03%), with subsequent neutralization with complete medium. Subsequently, the cells were transferred to conical tubes and centrifuged at 1000 rpm for 5 minutes. The pellet was resuspended in culture medium and cells were stained with 0.4% trypan blue dye for later counting and determination of cell viability in a TC20™ automatic counter (BioRad, Hercules, CA, USA). Cell viability values greater than 90% were expected.

As the molecular targets of ASMO APT20TTMG are located inside the nucleus of cells, APT20TTMG is taken up by the cells to initiate its proposed action. The cellular uptake of oligonucleotides is not well understood, but it is expected to occur in two steps: adsorption and internalization. Adsorption of antisense oligonucleotides modified with phosphorothioate to the cell surface is thought to be rapid and does not require energy. After adsorption, different endocytic pathways can internalize this type of oligonucleotide, including micropinocytosis. Cell-surface proteins can direct the oligonucleotides internationalization via non-conventional endocytic pathways or via clathrin- or caveolin-dependent endocytic pathways. Moreover, different cell types have differing oligonucleotides-binding abilities, and rapidly growing cells, including malignant cells, take up oligonucleotides more efficiently than slow-growing cells.

To investigate the internalization of ASMO APT20TTMG by MCF-7 cell line, the quantification of intracellular fluorescence was measured by flow cytometry. For this aim, the ASMO APT20TTMG was conjugated to the fluorochrome carboxyfluorescein. It is assumed that the percentage of ASMO molecules internalized is presumably proportional to the quantified fluorescence signal detected inside the cells. The cell lines were cultured in 24-well plates for 24 hours at 5×10⁴ cells per well. After that, the cells were incubated with ASMO1 prepared in culture medium without FBS at a concentration of 1 μM for 0.5, 1, 2, 4, and 6 hours. Untreated cells were used as negative controls. After treatment incubation, cells were washed twice with 1 mL of PBS/well and then incubated with 200 μL/well of trypsin/EDTA solution for approximately 3 minutes for disaggregation of adherent cells. Trypsin was neutralized by the addition of complete medium and cells were centrifuged at 1500 rpm for 5 minutes. Subsequently, the cells were washed with 2 mL/sample of PBS and centrifuged again at 1500 rpm for 5 minutes. Finally, the supernatant was discarded, and the cells were resuspended in 200 μL of PBS. The fluorescence of the cells, corresponding to the cellular uptake of the APT20TTMG labeled with carboxyfluorescein, was analyzed using a BD FACSCANTO II Flow cytometer (BD Biosciences, NJ, USA) through the acquisition of 10.000 gated events considering each sample.

MTT assay was carried out to assess the cell viability of MCF-7 cell line after treatment with ASMO APT20TTMG, and, therefore, infer the ASMO1 cytotoxicity. The tetrazolium salt reduction (MTT) method evaluates cell viability by assessing mitochondrial integrity. Viable cells are able to reduce MTT reagent, which is yellow and water-soluble, into an insoluble purple formazan product. Formazan quantification could be measured by a spectrophotometer absorbance at 560 nm. The quantity of formazan is directly proportional to the number of viable cells in the culture.

For this assay, MCF-7 cells were plated in 96-well plates at a density of 1×10⁴ cells/well and cultured for 24 hours for adhesion and, subsequently incubated with eight concentrations (0.0078-1 μM) of APT20TTMG (prepared in medium with 2% FBS) for the period of 48 hours. Then, cells were washed with 150 μL/well of PBS and incubated with 100 μL/well of MTT solution prepared in culture medium, at a concentration of 0.5 mg/mL for 3 hours in the incubator. After that, the MTT solution was removed from the plate and the produced formazan crystals were solubilized with 100 μL/well of Dimethylsulfoxide (DMSO) and mixed in an orbital shaker at 300 rpm for 20 minutes. Finally, the absorbance of the samples was measured in a plate reader spectrophotometer (Multiskan Spectrum, Thermo Scientific, Waltham, MA, USA), at a wavelength of 560 nm. The absorbance of each sample was used to determine the cell viability after each treatment condition.

The cell cycle consists of DNA replication (S phase), mitosis (M), and cytokinesis, separated by two gaps (G1 and G2). A non-proliferative state, known as G0 (or quiescence), could also happen during G1 phase. These gap phases are important for cell cycle regulation and decisions related to, in G1 phase, enter in the cell cycle or, in G2 phase, initiate the process for segregation of the DNA. In this way, cell cycle progression regulates cell proliferation, and its dysfunction has a key role in the development of cancer, mainly in malignant tumors. The control of cell cycle progression by triggering cell cycle arrest may be an important feature of cancer therapy.

Cell cycle assays were performed to understand how the ASMO APT20TTMG affects the cell cycle of MCF-7 cell line and its potential mechanisms of action. Initially, cells were plated in 12-well plates at a density of 5×10⁴ cells/well and cultured for 24 hours for adhesion and then treated with three different concentrations of APT20TTMG (0.25, 0.5 and 1.0 μM) for 48 hours. After, cells were collected using a solution of trypsin/EDTA and washed with 2.0 mL of PBS buffer followed by centrifugation at 1500 rpm for 5 minutes. Then, the cells were fixed with ice-cold 70% ethanol and incubated overnight at 4° C. After that, cells were washed again with 2.0 mL of PBS and incubated with Propidium Iodide solution (50 μg/mL) and RNAse (200 μg/mL) for 1 hour at room temperature and protected from light. Finally, the cells were analyzed in a flow cytometer (BD FACSCanto II, BD Biosciences), through the acquisition of 10.000 gated events considering each sample.

Although there are several forms of cell death, with different pathways, the two distinct processes, apoptosis (also known as programmed cell death) or necrosis (uncontrolled cell death) still emerge as the main ones. Apoptosis is characterized by several characteristic morphological changes in the structure of the cell, together with several enzyme-dependent biochemical processes. The result is the clearance of cells from the body, with minimal damage to surrounding tissues. Cancer cells often evade apoptosis by expressing proteins that inhibit the apoptotic process or by activating signaling pathways that promote cell survival. In addition, cancer cells often exhibit abnormalities in the signaling pathways that control the cell cycle and cell proliferation, which can contribute to their uncontrolled growth and proliferation. Dysregulation of the apoptotic process can contribute to the development and progression of cancer, but at the same time, inducing apoptosis in cancer cells can be an effective strategy for cancer treatment.

The apoptosis-inducing potential of ASMO APT20TTMG was evaluated using the method of double labeling of cells with Annexin V and propidium iodide. Annexin V is a peptide that has a high affinity for phosphatidyl serine, a phospholipid distributed asymmetrically in the inner leaflet of the lipid bilayer of membrane plasma under physiological conditions. During the process of cell death by apoptosis, phospholipids asymmetric distribution is lost and phosphatidyl serine is translocated to the outer leaflet of the plasma membrane through a process of externalization. The measurement of the exposure of phosphatidylserine could be detected by Annexin V conjugated to fluorochromes. On the other hand, propidium iodide is a DNA intercalator that only can penetrate the interior of the cell in cases where the selective permeability of the plasma membrane is compromised, which clearly occurs during several processes of cell death.

Pro-apoptotic potential of APT20TTMG was evaluated in the MCF-7 cell line, using the Apoptosis Detection with Annexin V/Propidium Iodide kit (eBioScience). First, cells were cultured in 24-well plates at a density of 1×10⁵ cells/well for 24 hours for adhesion. Then, cells were treated with three concentrations of APT20TTMG (0.25, 0.5 and 1p M) for 24 hours and then collected with a solution of 0.05% trypsin. After the treatment, cells were washed once with 2 mL of ice-cold PBS and once again with the binding buffer provided by the kit. The samples were labeled with Annexin V conjugated to FITC for 15 minutes and washed again with the binding buffer. Finally, the samples were labeled with Propidium Iodide and analyzed in a flow cytometer (BD FACSCanto II, BD Biosciences) through the acquisition of 10.000 gated events/sample.

Mitochondria are essential for the cell's survival, and, in cancer, could contribute to the uncontrolled growth and proliferation of these cells. Mitochondria in cancer cells have a different function and structure than the mitochondria in normal cells. One of the main differences is that cancer cells have altered membrane potential, when compared to healthy cells, and produce energy for the cell in a different way. Mitochondrial membrane potential represents the difference in electrical charge across the inner membrane of the mitochondria and is essential for ATP synthesis by oxidative phosphorylation. Mitochondrial membrane potential abolition is an early event during apoptosis, which is a type of cell death common tumor cells responding to treatment.

The evaluation of ASMO1 APT20TTMG effect on the mitochondrial membrane potential of MCF-7 cell line was performed using MitoTracker Red dye. MitoTracker Red is a cationic fluorophore that accumulates in the mitochondria matrix, providing an indication of the viability and integrity of that organelle.

For this, cells were cultured overnight in 24-well plates at a density of 1×10⁵ cells/well, aiming cell adhesion. After that, cells were treated with three concentrations of APT20TTMG (0.25, 0.5, and 1 μM) for 24 hours. After treatment with the ASMO1, cells were collected using a 0.05% trypsin solution and washed twice with 2.0 mL of PBS buffer. Finally, samples were incubated with 500 μL of MitoTracker Red fluorescent dye solution at a concentration of 50 nM for 15 minutes in an incubator. Then the cells were again washed twice with 2 mL of PBS and, subsequently, analyzed in a flow cytometer (BD FACSCanto II, BD Biosciences) through the acquisition of 10.000 gated events/sample.

Values were expressed as mean±standard error of the mean. To assess the statistical difference between the groups, the one-way ANOVA test was performed followed by the Dunnett's multiple comparison test. P values less than 0.05 were considered as indicative of significance. GraphPad Prism 9 software (GraphPad Software Inc., San Diego, CA, USA) was used for statistical analyses.

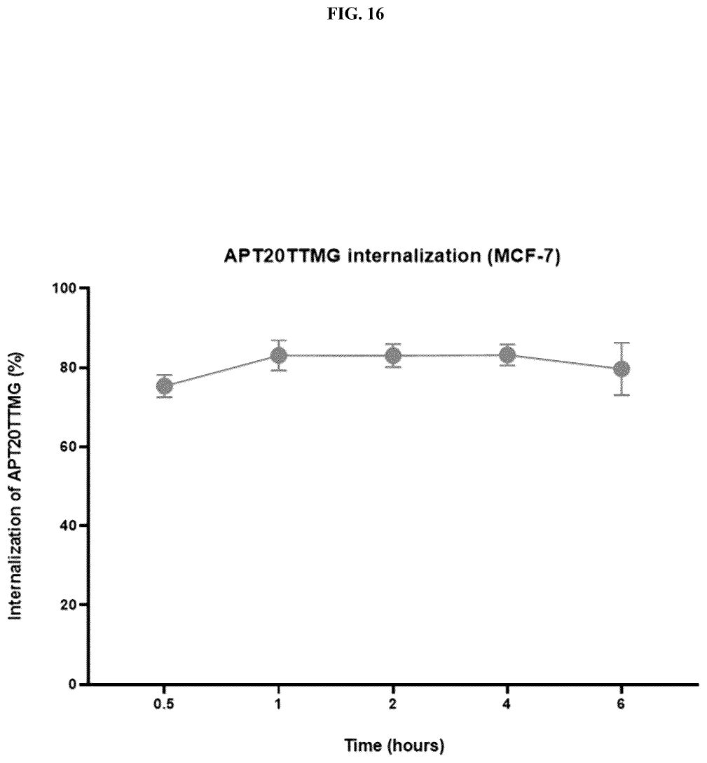

As shown in FIG. 16, the internalization values of ASMO APT20TTMG were quite high, showing 75.3±1.4% in the first half hour after incubation with the MCF-7 cell line. After 1 hour of incubation, the degree of internalization remained constant during the subsequent evaluation period, with approximate values around 80%.

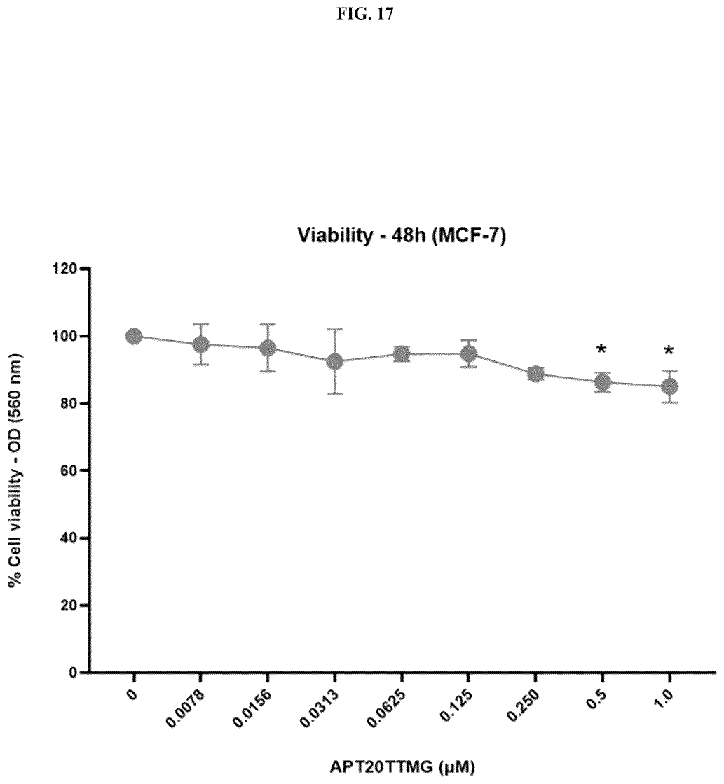

As shown in FIG. 17, after 48 hours of incubation, there was a reduction in cell viability at concentrations of 0.5 and 1 μM, which showed 86.4±1.6% (p=0.0235) and 85±2.7% (p=0.0120), respectively. After determining the concentration range that may have an antitumor effect, new tests were performed for concentrations of 0.25, 0.5 and 1 μM. This assessment of cell viability may represent mitochondrial activity, but also, indirectly, cell proliferation. In the two highest concentrations tested, there was probably a reduction in the number of cells.

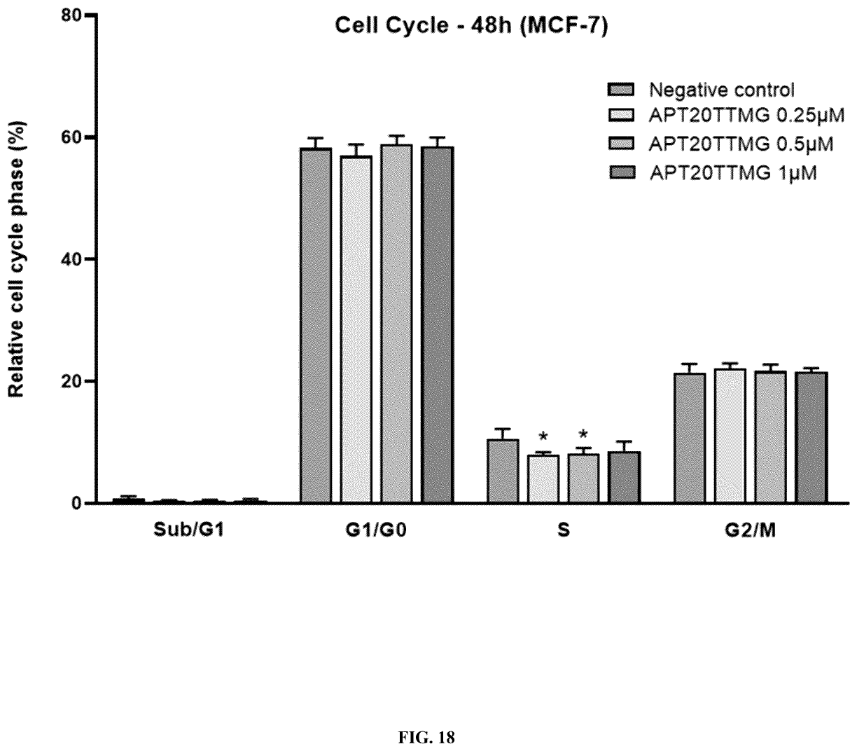

As shown in FIG. 18, the only statistically significant alteration was in the S phase, that is, in the phase where DNA synthesis occurs, there was a lower concentration of cells, indicating that they were not retained in this phase, after incubation with 0.25 and 0.5 μM.

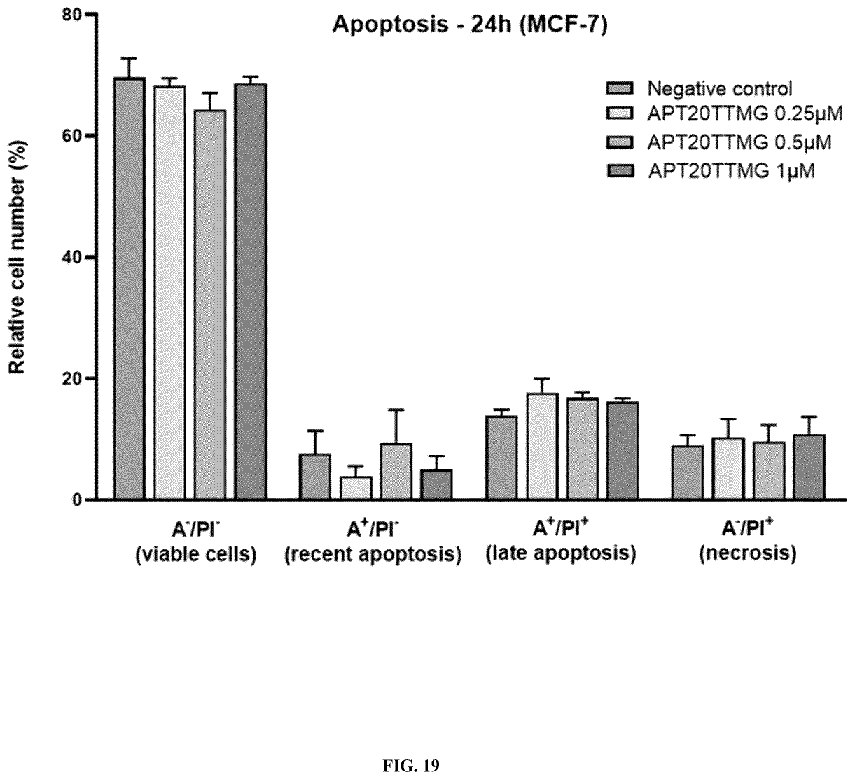

As shown in FIG. 19, there was no change in any of the evaluated parameters, that is, all treatments showed a similar profile to the negative control (non-treated cells) in relation to the proportion of viable cells, in recent/late apoptosis or necrosis.

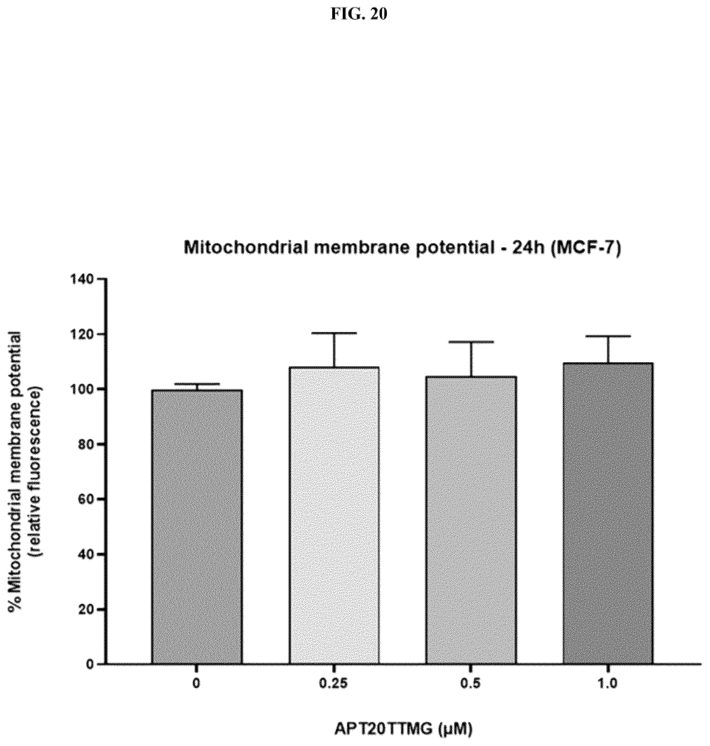

As shown in FIG. 20, there was no change in mitochondrial membrane potential, that is, all treatments showed a similar profile to the negative control (non-treated cells).

Upon completion of the study, it was found that ASMO1 was efficiently internalized by the carcinoma breast cell line, MCF-7. Besides, it showed reduced cell viability at higher concentrations, which may be related to reduced cell proliferation. The results show that there was no change in mitochondrial membrane potential and apoptosis, however APT20TTMG appears to reduce the number of cells in the S phase of the cell cycle.

### Example 4. Evaluation of the Antitumor Potential of ASMO1 APT20TTMG in Neuroblastoma Cell Lines (SH-SY5Y and SK-N-SH)

In physiological conditions, TAU protein is a phosphoprotein that promotes microtubule assembly and its stabilization in cells. TAU also has important roles in chromatin structure, signal transduction, and nucleic acid protection. In fact, this protein is described to have a wide interactome, including interaction with cancer-related kinase proteins, PI3K/AKT (associated with cell survival and proliferation in many cancers), and Rho-ROCK signaling (involved in cell migratory and invasive phenotypes). Consequently, despite being largely known for its role in neurodegenerative diseases, recent studies show that TAU could also be involved in the progression of many cancers as well as in cell migration and invasiveness. Furthermore, as a microtubule-binding protein, TAU may interfere with the binding of taxanes (microtubule-stabilizing drugs) to tubulin, therefore, being involved in the resistance to taxanes used in cancer treatment. In this way, anti-TAU molecules could also be a strategy to improve the effect of taxane-based chemotherapies. Therefore, the modulation of TAU expression, both at mRNA and protein level, could be used for cancer chemotherapy.

Cells were rinsed once with ice-cold PBS, collected, and pelleted by centrifugation at 2500 g for 5 minutes at 4° C. Each sample was re-suspended and lysed in 50 μl ice-cold radioimmunoprecipitation assay (RIPA) buffer (Thermo Fisher Scientific, USA) containing EDTA-free Halt protease and phosphatase inhibitor cocktail mixture (Thermo Fisher Scientific, USA) for 20 min at 4° C. To increase protein yields, each sample was homogenized using a 29 G×13 mm needle and syringe. Cell lysates were clarified by centrifugation at 17,000 g for 15 minutes at 4° C. Supernatants were collected and total protein concentration was determined using the BCA protein assay kit (Thermo Fisher Scientific, USA) according to the manufacturer's instructions. 15 μg of total protein in cell lysates were denatured in NuPAGE LDS sample buffer (Thermo Fisher Scientific, USA) with NuPAGE sample reducing agent (DTT; Thermo Fisher Scientific, USA) and boiled at 90° C. for 5 minutes. Samples were separated on NuPAGE 4-12% Bis-Tris gels (Thermo Fisher Scientific, USA) using the XCell SureLock Mini-cell system (Thermo Fisher Scientific, USA), and electrophoresed at 120 V in NuPAGE MES SDS running buffer (Thermo Fisher Scientific, USA). Resolved proteins were transferred to polyvinylidene difluoride (PVDF) 0.45 μm membranes (Thermo Fisher Scientific, USA) at 120 V for 75 minutes at 4° C. in NuPAGE transfer buffer (Thermo Fisher Scientific, USA) containing 20% methanol. PVDF membranes were blocked for 1 hour at room temperature in TBS-T with 5% (w/v) dried milk powder and then incubated overnight at 4° C. with total TAU primary antibody (Cell Signaling Technology, Cat #46687 or Abcam, Cat #ab80579), diluted in TBS-T with 2% (w/v) BSA. Membranes were then washed in TBS-T before incubation with HRP-conjugated secondary antibody at a dilution of 1:5000 in TBS-T for 1 hour at room temperature. Membranes were washed in TBS-T once again and proteins were visualized with enhanced chemiluminescence (ECL) reagent (Thermo Fisher Scientific, USA) and images were captured using a Jess Simple Western Imaging suite. Densitometry using Image J (v1.48k; NIH) was performed and all quantification was standardized against vinculin levels.

At all time points cells were rinsed once with PBS, before proceeding with RNA extraction using the RNeasy Plus Kit (Qiagen). RNA (0.1-1 μg) was converted to cDNA using the SuperScript IV First-Strand Synthesis System (Thermo Fisher), following manufacturer instructions. After RNA removal, 5-10 ng of cDNA were amplified by qPCR. The following Taqman probes (Thermo Fisher) were used: Total TAU (MAPT, Assay ID: Hs00902193_m1), GAPDH (GAPDH, Assay ID: Hs99999905_m1). Gene expression was analyzed using the Applied Biosystems QuantStudio™ 12K Flex Real-Time PCR System. Quantification of gene expression was performed using the comparative threshold cycle (2\hat{ }-ΔΔCT) Livak method, relative to Lipofectamine 3000 only treated cells. Gene expression was normalized to the reference gene GAPDH.

Values were expressed as mean±standard error of the mean. To assess the statistical difference between the groups, the one-way ANOVA test was performed followed by the Dunnett's multiple comparison test. Western Blot and qPCR analysis of final experiments were carried out with a two-tailed t-test, and p-values<0.05 were considered statistically significant, compared to MOCK (cells treated with lipofectamine 3000 only control). GraphPad Prism 9 software (GraphPad Software Inc., San Diego, CA, USA) was used for graphics generation.

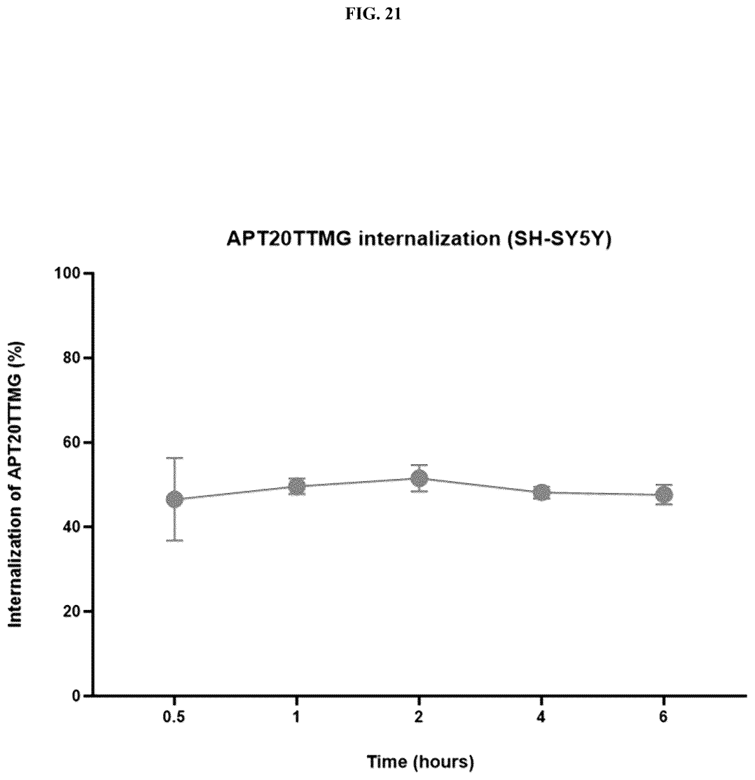

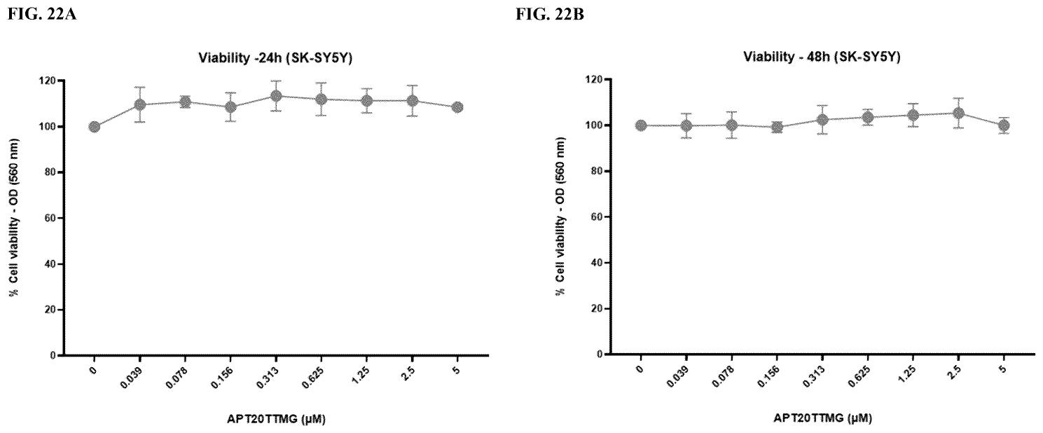

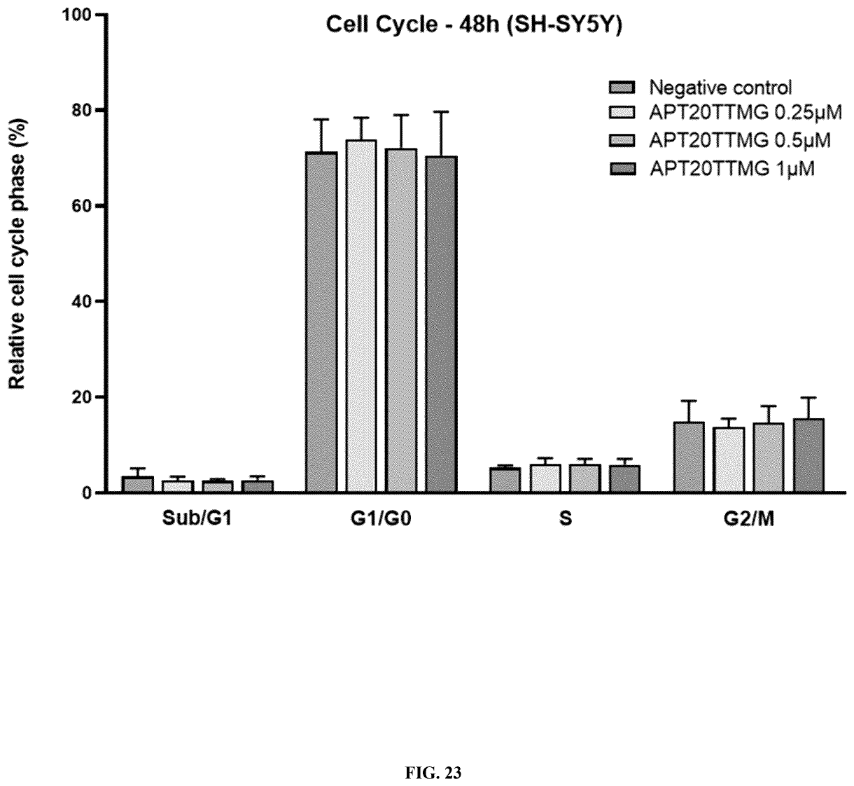

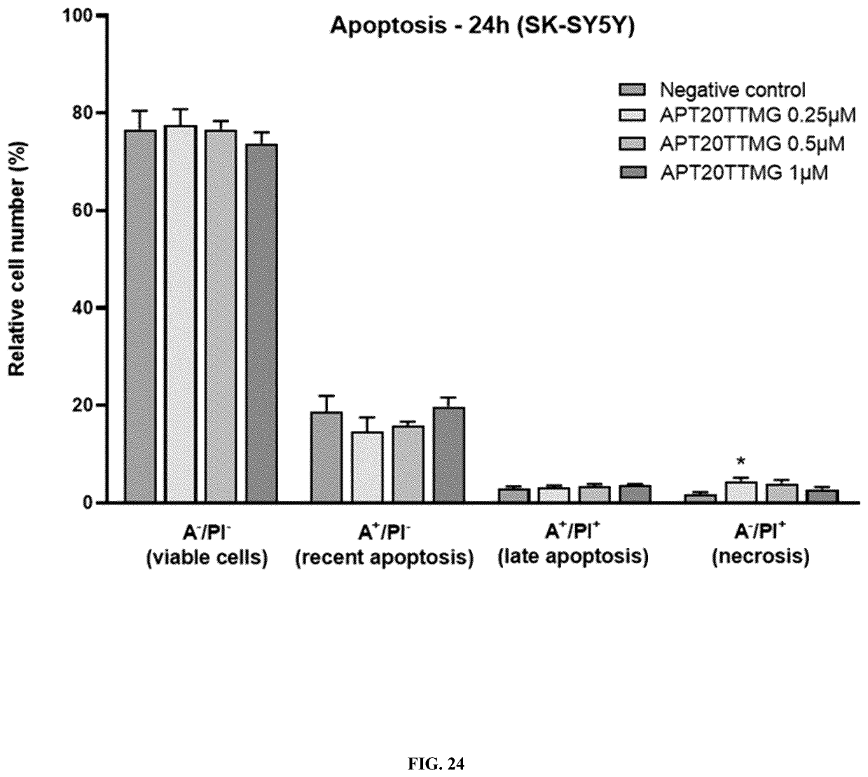

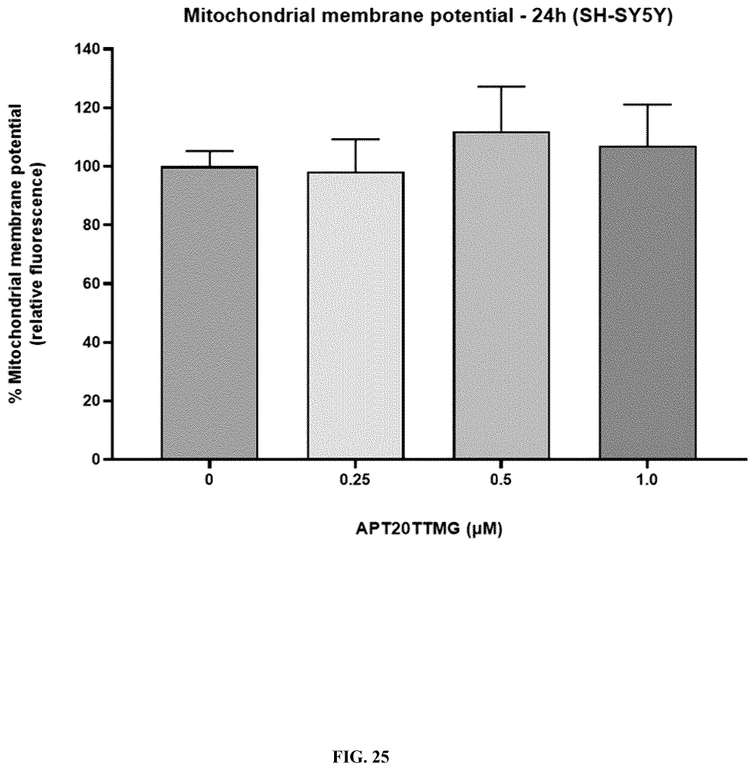

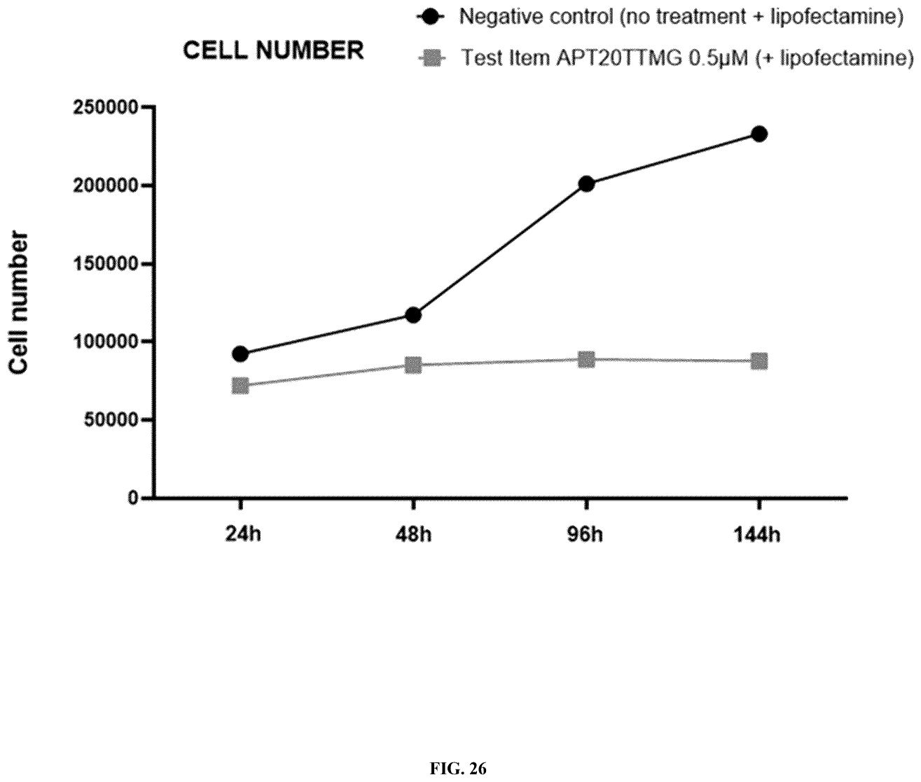

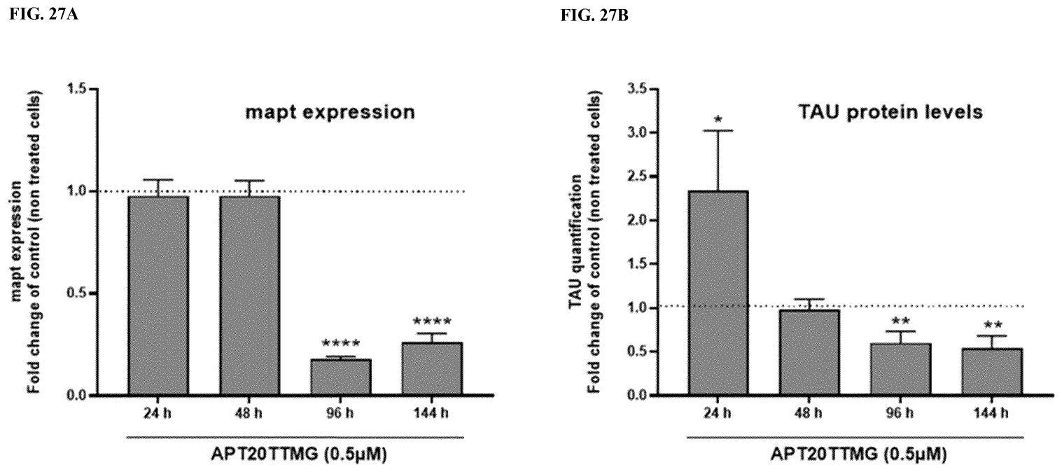

As shown in FIG. 21, there was internalization of ASMO1 APT20TTMG, presenting around 50% for all evaluated times. As shown in FIG. 22A-B, there was no change in cell viability at any concentration and times evaluated. As shown in FIG. 23, there was no change in cell cycle phases at any concentration evaluated. As shown in FIG. 24, there was an increase in necrotic cells after 24 hours of incubation with ASMO1 APT20TTMG (0.25 μM). As shown in FIG. 25, there was no change in mitochondrial membrane potential, that is, all treatments showed a similar profile to the negative control (non-treated cells). As shown in FIG. 26, after 0.5 μM of APT20TTMG ASMO1, the number of viable cells remains practically the same over the 4 times analyzed, indicating a cytostatic effect. As shown in FIG. 27A-B, except for the transient increase observed at 24 hours, regarding the regulatory profile of MAPT expression in the two longest times, there was a reduction of 82±1.2% and 74±4.3%, for the times of 96 and 144 h, respectively. Similar profile was observed for the two highest concentrations in protein quantification values, reducing by 40±13.3% and 46±14.3%.

ASMO1 is internalized by the neuroblastoma cell line, SH-SY5Y. However, there was no change in cell viability or proliferation, cell cycle, and mitochondrial membrane potential. Cytotoxicity or cytostatic potential may be indicated based on a slight change in necrotic cells.

Regarding SK-N-SH neuroblastoma cell line, APT20TTMG has the potential to reduce TAU protein levels and MAPT gene expression in a time dependent manner and the number of viable cells remains practically the same over the 4 times analyzed (cytostatic effect).

### Example 5. Evaluation of the Antitumor Potential of ASMO APT20TTMG in Glioblastoma Cell Line (U87-MG)

The methodologies for this Example are the same as shown in Example 4. Since the preliminary study with 5 different human tumor lines showed antitumor potential after incubation with the ASMO1, the three most promising lines went on for further evaluation, including glioblastoma.

In vitro effects of APT20TTMG on viability and proliferation of human cancer cell line U87MG (Human Glioblastoma) were also evaluated by MTT and BrdU assays, respectively. Cells were treated with APT20TTMG and Reference Items in technical triplicates (Experiment was done in 3 different sets) at various concentrations for 48 h, 96 h, and 144 h. APT20TTMG and Reference Items were read after every 48 h.

Values were expressed as mean±standard error of the mean. To assess the statistical difference between the groups, the one-way ANOVA test was performed followed by the Dunnett's multiple comparison test. GraphPad Prism 9 software (GraphPad Software Inc., San Diego, CA, USA) was used for graphics generation.

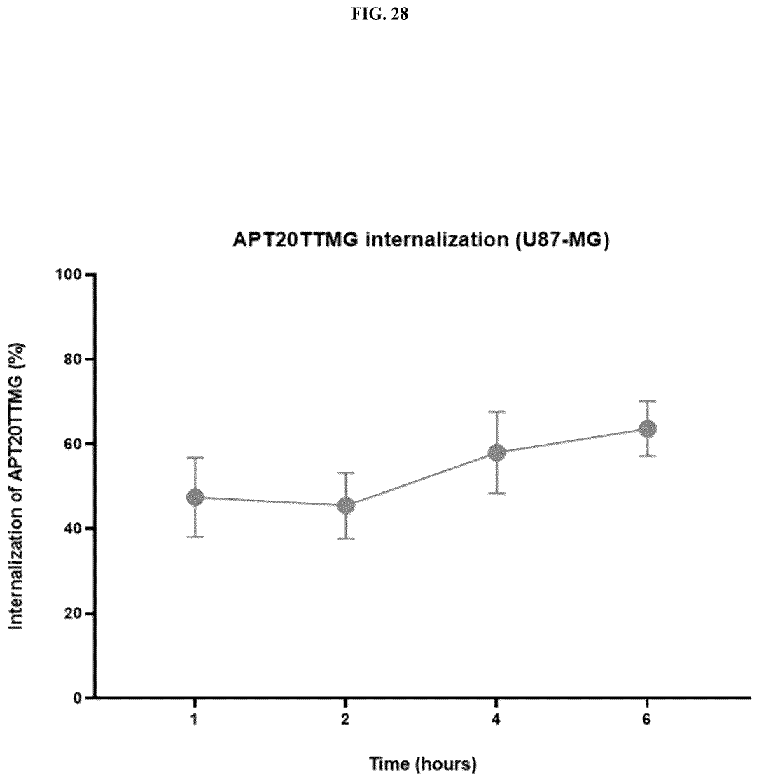

As shown in FIG. 28, internalization of the ASMO1 is increased in the longest evaluated times. The values obtained were: 1 hour (47.45±4.7%), 2 hours (45.45±3.9%), 4 hours (58±4.8%) and 6 hours (63.65±3.2%). Cells were exposed to the fluorochrome-conjugated compound FAM at a concentration of 1 μM, so that the quantification of intracellular fluorescence was performed at 1, 2, 4 and 6 hours using of the flow cytometry technique, through the acquisition of 10,000 gated events. Data of 2 independent experiments performed in two technical replicates.

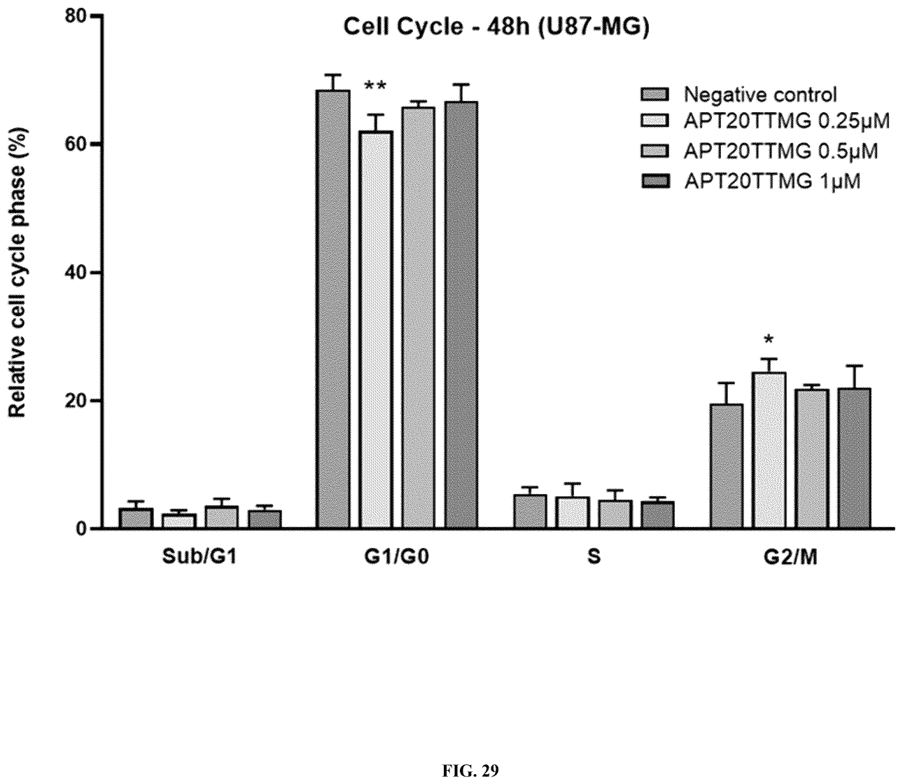

As shown in FIG. 29, APT20TTMG showed significant changes in two phases of the cell cycle: phase G1/G0 and G2/M at a concentration of 0.25 μM. In the first, there was a reduction in retention (p=0.0036) and an increase in retention in the G2/M phase (p=0.0425), suggesting that the ASMO1 prevented the proliferation of cells from this cell line. Cells were plated and treated with 3 different concentrations (0.25, 0.5, and 1 μM) of APT20TTMG and cell cycle phases were evaluated after 48 hours of incubation. Cell proliferation is regulated by cell cycle progression. Phases: Sub/G1: non-proliferative state (quiescence); G1/G0: cell growth; S: DNA replication; G2/M: DNA segregation and mitosis. Results were expressed as Mean Standard Error of three independent experiments.

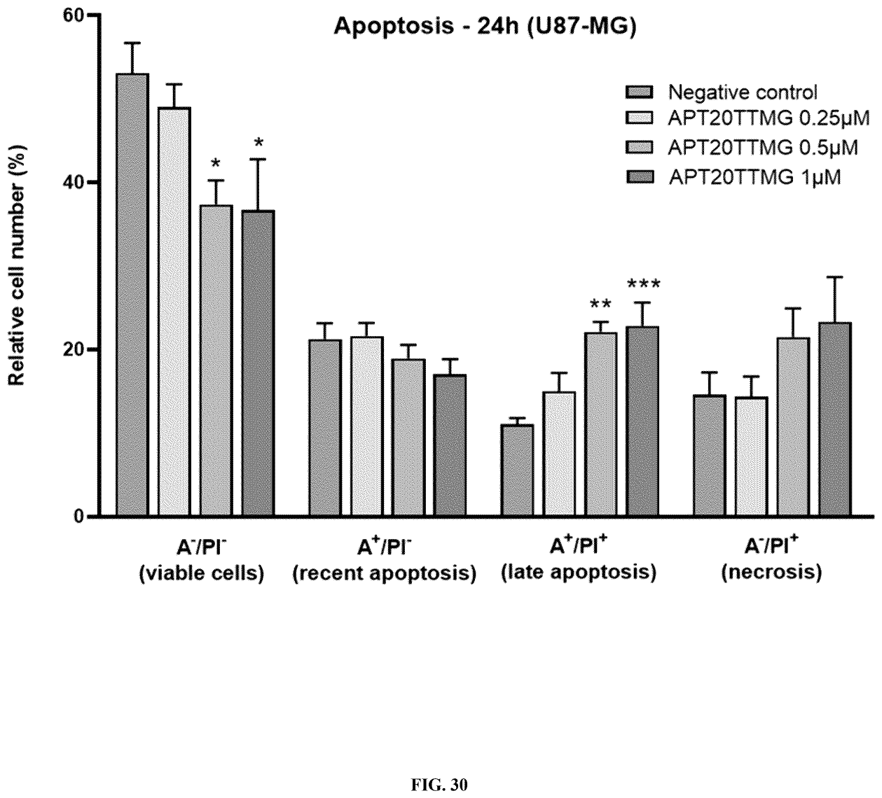

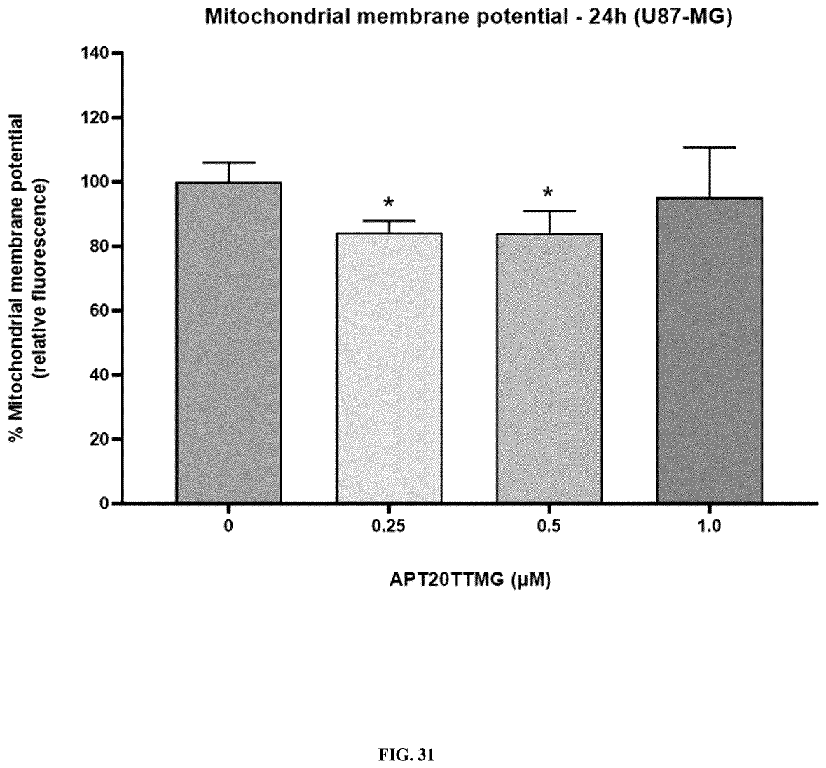

As shown in FIG. 30, the apoptosis assay after incubation with APT20TTMG show that the two highest concentrations reduce cell viability and are significantly marked for late apoptosis, while 0.25 μM concentration just shows a trend toward this state. As shown in FIG. 31, in addition to the results obtained in the cell cycle and apoptosis, the ASMO1 also changes the mitochondrial membrane potential, reducing the labeling for the concentrations of 0.25 and 0.5 μM. Cells were plated and treated with 3 different concentrations (0.25, 0.5, and 1 μM) of APT20TTMG and cell cycle phases were evaluated after 48 hours of incubation. Cell proliferation is regulated by cell cycle progression. Phases: Sub/G1: non-proliferative state (quiescence); G1/G0: cell growth; S: DNA replication; G2/M: DNA segregation and mitosis. Results were expressed as Mean±SEM of three independent experiments.

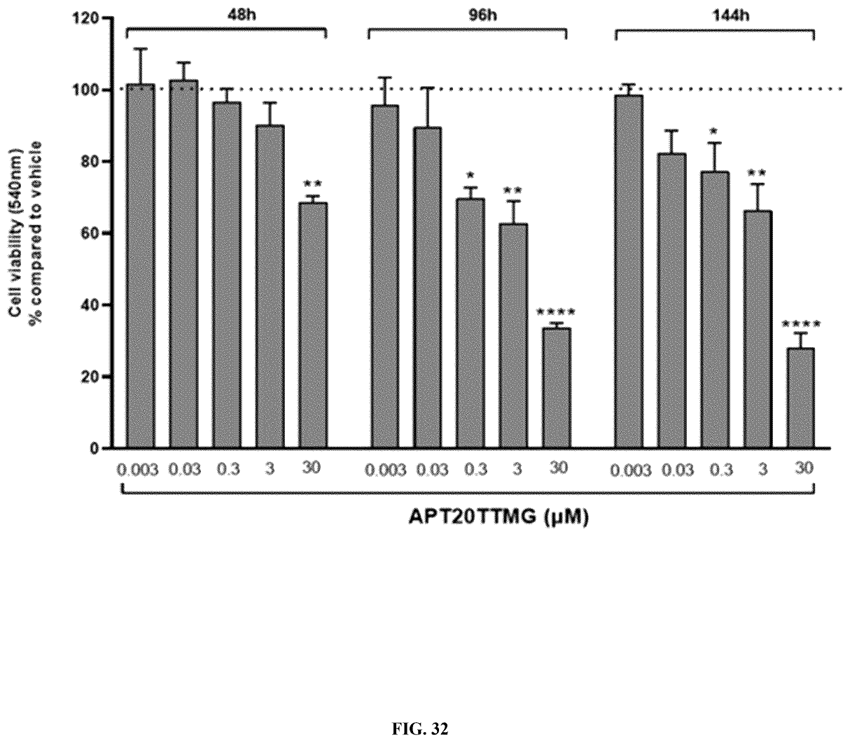

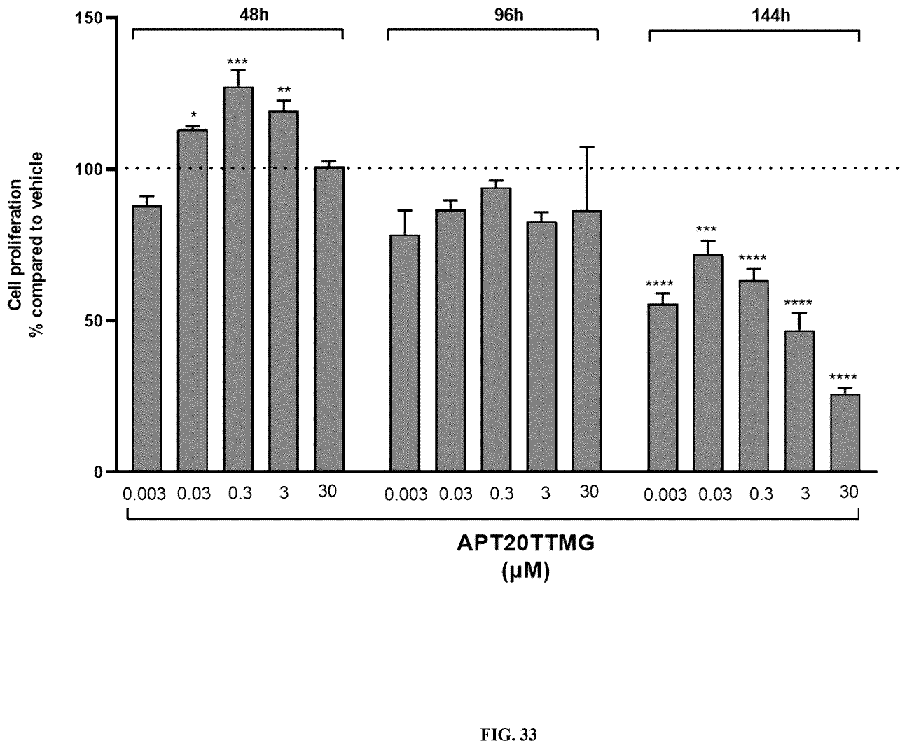

As shown in FIGS. 32-33, A MTT assay (FIG. 32) was also performed with another five different concentrations of APT20TTMG (0.003, 0.03, 0.3, 3, and 30 μM) and at 48, 96, and 144 hours of incubation. After 48 hours of incubation, only the 30 μM concentration of APT20TTMG was observed to reduce cell viability, while, after 96 and 144 hours of incubation, the three highest concentrations of APT20TTMG (0.3, 3, and 30 μM) also showed this capacity, suggesting a cytotoxic and/or cytostatic potential. Finally, to confirm whether APT20TTMG is a cytotoxic or cytostatic molecule considering its ability to reduce cellular viability in certain concentrations, a cell proliferation assay (FIG. 33) with the U87-MG cell line was also performed in the same conditions as the MTT assay. At the lowest concentrations (0.003 and 0.03 μM) of APT20TTMG, despite the increase in cell proliferation observed in the first 48 hours of incubation at the 0.03 μM concentration, a statistically significant decrease in proliferation is observed in the last 144 hours of the assay. This decreased profile along with the absence of cell viability alteration indicates that APT20TTMG is cytostatic at its lowest concentrations (0.003 and 0.03 μM). At the highest concentrations of APT20TTMG (0.3, 3, and 30 μM), besides the temporary increase in cell proliferation observed in the first 48 hours of the assay, a significant reduction in cell proliferation is observed by the 144 hours of incubation. At the highest concentrations of APT20TTMG (0.3, 3, and 30 μM), in addition to the transient increase in cell proliferation observed in the first 48 hours of the assay (which could be related to a beneficial increase in astrocytes number in an astrogliosis activated pathway), a significant reduction in cell proliferation is observed in the 144 hours of incubation. This data (FIG. 33) along with the MTT results (FIG. 32) indicate that APT20TTMG is cytotoxic at the highest tested concentrations. Cells were plated and treated with 5 different concentrations (0.003, 0.03, 0.3, 3, and 30 μM). BrdU assay was performed after 48, 96, or 144 hours of incubation. Data are plotted as a percentage (%) of cell viability compared to the negative control group (vehicle). Results are expressed as Mean±SEM of three independent experiments performed in three technical replicates. Statistical analysis was performed using one-way ANOVA followed by Dunnett's multiple comparison test. *p<0.05, **p<0.01, ***p<0.001, ****p<0.0001.

The results obtained from the incubation with the ASMO1 in glioblastoma cell line U87-MG strongly suggest antitumor potential, since they showed a reduction of viable cells and mitochondrial membrane potential, in addition to increasing the retention of cells in the G2/M phase, preventing the progression of cells to divide. Furthermore, APT20TTMG enhances the labeling for cells in late apoptosis. Overall, the in vitro results with GBM cell line (U87-MG) indicate that APT20TTMG is efficiently internalized and has cytotoxic and cytostatic profiles depending on the tested conditions.

### Example 6. Evaluation of In Vivo Anti-Tumor Potential of APT20TTMG Using Human Glioblastoma Cancer (U-87MG) Xenograft Model in Athymic Nude Mice

Subcutaneous inoculation of the tumorigenic Human Glioblastoma Cancer Cell line (U-87MG) results in the formation of solid tumor in athymic nude mice. Under pathogen free conditions, the tumor retains its own characteristics and grows using the growth factors supplied by the host animal. The ability of administered ASMO1 to inhibit the growth of tumor can be a valuable tool to evaluate its efficacy as an anti-tumor agent. Measurement of tumor size at defined intervals of time generates data that can be used to calculate various end point parameters derived from tumor and understand the potency of the test compounds.

Human glioblastoma cancer cells (U-87MG) were cultured and expanded in EMEM (Eagle's Minimum Essential Medium) supplemented with 10% Fetal Bovine Serum (FBS) at 37° C. and 5% CO₂. The cells growing at log phase were harvested by trypsinization and centrifuged for 3 minutes at 800 rpm to form a pellet. The pellet was further washed with PBS. Cell suspension stock was prepared to obtain the required cell count. Before cell inoculation, cell viability was determined using trypan blue. 25 μL of cell suspension was stained with 25 μL of trypan blue and viable cells were counted using Neubauer's chamber. Cell number was adjusted by adding serum free media to achieve desired concentration, i.e., 10×10⁷/mL.

Cells were diluted such that the concentration of the cells was 10 million cells/100 μL of serum free medium. Overnight thawed Matrigel and cells with medium were mixed 1:1 and a total volume of 200 μL was injected subcutaneously to the donor animals (n=5) at flank region.

Donor animals were monitored for solid tumor growth for at least 3-4 weeks. Tumor size was measured using digital caliper (MITUTOYO, Japan, CP-15Mx) and volume was calculated by using the formula: (L*W2)/2. Once the tumor reached required size ˜500 mm³, the donor animals were humanely sacrificed, the tumor was harvested and fragmented into ˜30 mg size and implanted subcutaneously to experimental animals (n=50) using the device. All the animals was monitored alternate day for tumor growth/size.

Once the tumor size reached ˜100 mm³, animals were selected and randomized by stratification method using tumor size into three groups (G1-G3). Treatment for G1-G3 was initiated at this stage. Each group was comprised of 8 animals.

TABLE 6
Experimental design

| Group | Treatment | Dose, Route & Regimen | No of animals |
|---|---|---|---|
| G1 | Normal Saline | 5 mL/kg, i.v., qdx21 | 8 |
| G2 | Reference Item: | 5 mg/kg, p.o., qdx21 | 8 |
| | Temozolomide | | |
| | (TMZ) | | |
| G3 | TEST ITEM: | 20 mg/kg, i.v., q3dx7 | 8 |
| | APT20TTMG | | |

G1 was served as vehicle control group and treated with normal saline at the dose of 5 mL/kg, daily, intravenously for 3 weeks.
G2 was served as Reference Item treated group and treated with TMZ at the dose of 5 mg/kg, p.o., daily for 3 weeks.
G3 was treated with APT20TTMG at the dose of 20 mg/kg, intravenously, every third day (q3d) for 3 weeks.

Body weight and tumor size were measured weekly twice using digital caliper. At termination, blood was collected through retro-orbital route. Serum/plasma was separated and stored at deep freezer (−20° C.). Post blood collection, animals were humanely sacrificed by CO₂ asphyxiation as per latest guidelines issued by CPCSEA. All the vital organ (brain, kidney, liver, lungs, heart) and tumor were dissected out. Vital organs were fixed in 10% NBF and stored. Tumor tissue was dissected out, weighed and portion of tumor tissue was fixed in 10% NBF and another portion is snap frozen and stored at deep freezer (−80° C.).

The fixed tissues were processed for routine paraffin embedding followed by sectioning (3-6 micron). The sections were stained with hematoxylin and eosin. The prepared slides were examined under microscope by the study pathologist to record histopathological findings in tumor samples, related to capsule formation, cell type, connective tissue stroma, apoptosis, necrosis, lipid content, nucleus-to-cytoplasmic (N:C) ratio, nuclei mitotic figures, tumor grade, and level of differentiation. Each histopathological finding and tumor volume were assigned a score ranging from 1 to 4 (Table 7). The sum of these scores for each sample was used to infer the tumor aggressiveness.

TABLE 7
Histopathology score of tumor samples

| Histopathological criteria | Description | Score |
|---|---|---|
| Connective tissue stroma | Minimal Fibrosis | 1 |
| | Mild Fibrosis | 2 |
| | Moderate Fibrosis | 3 |
| | Prominent | 4 |
| Apoptosis | Minimal | 4 |
| | Mild | 3 |
| | Moderate | 2 |
| | Prominent | 1 |
| Lipid content | Prominent | 4 |
| | Moderate | 3 |
| | Mild | 2 |
| | Minimal | 1 |
| Necrosis | Minimal | 1 |
| | Mild | 2 |
| | Moderate | 3 |
| | Prominent | 4 |
| N:C ratio | High | 3 |
| | Low to High | 2 |
| | Low | 1 |
| Mitotic figures | Few | 1 |
| | Moderate | 2 |
| | Large | 3 |
| | <500 | 1 |
| Volume Tumor | 500-1000 | 2 |
| | 1000-1500 | 3 |
| | >1500 | 4 |
| GRADE | I | 1 |
| | II | 2 |
| | III | 3 |
| | IV | 4 |

Abbreviation: N:C, nucleus-to-cytoplasmic.

Immunohistochemistry analysis of Ki67 in tumor samples was also performed. Quantification of the Ki67 in the sections was carried out by visual estimation (Ki67+ve) using two approaches: count of Ki-67 positive tumor cells and Ki67 total area. This process was done using ImageJ public domain software. In the case of the Ki67 total area evaluation, the ImageJ threshold function was used and each image was split into its constituent red, blue and green grayscale images. Further, one of these was selected and processed for threshold values. Values were selected so that the relevant areas (Ki67+ve cells, in this case) were highlighted and the % of area occupied by the selected threshold limits measured.

For the assessment of biomarkers such as cAMP, AKT, GFAP, and TAU by ELISA, the U-87MG tumor tissues dissected out from the animals were homogenized according to kit instructions. The homogenates were centrifugated, and the supernatant was collected and immunoassayed with the Human AKT Protein ELISA Kit (MyByoSouce), cyclic AMP Complete Enzyme-linked Immunosorbent Assay (ELISA) kit (MyByoSouce), Human Glial Fibrillary Acidic Protein (GFAP) ELISA Kit (Cusabio), and Human TAU Protein (TAU) ELISA Kit (MyByoSouce) for the quantitative determination of cAMP, AKT, GFAP, and TAU proteins, respectively.

Data on each parameter was summarized in both tabular and graphical form. Results of all parameters were expressed in Mean±SEM for all the groups or was expressed in median value. Comparisons were made between the groups using one-way ANOVA or two-way ANOVA followed by appropriate post-hoc test, except by the scoring evaluations of the histopathological findings and tumor volume, which were performed using Kruskal-wallis test followed by appropriate post-hoc test. Statistical analysis was conducted using PRISM software, Version 5.01. ‘p’ values of <0.05 was considered as statistically significant.

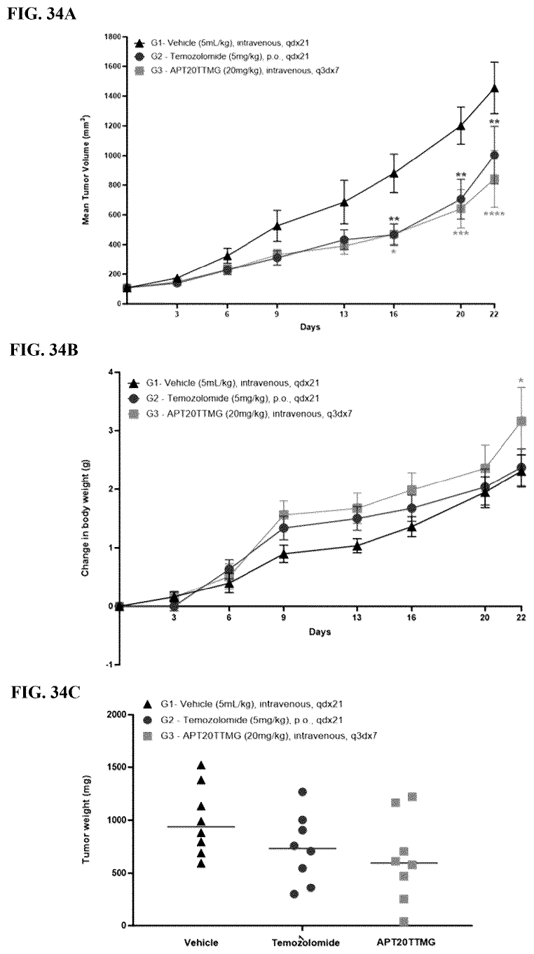

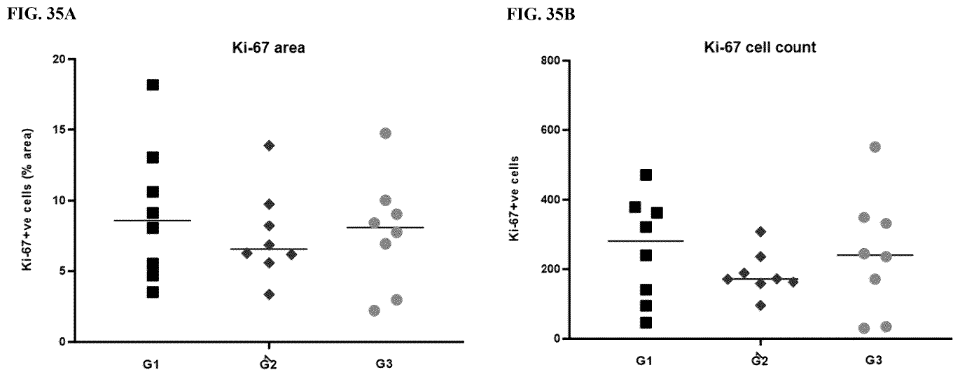

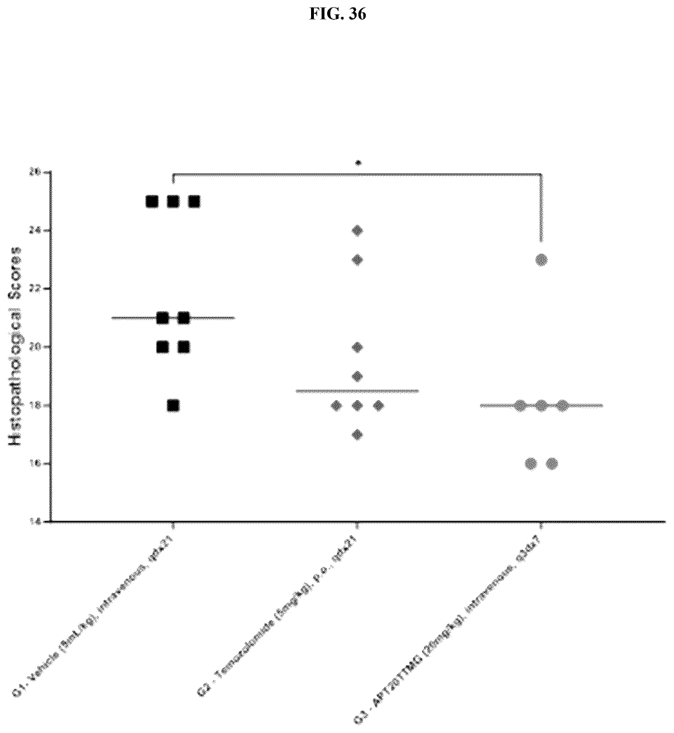

The evaluation of in vivo anti-tumor potential of APT20TTMG using human GBM cancer (U-87MG) xenograft model in athymic nude mice (FIG. 34) was important to confirm whether the antitumor effects of APT20TTMG also occur in an animal model and to verify the ability of APT20TTMG to penetrate and permeate the tumor in a xenograft animal model of GBM through an intravenous administration. When compared to the vehicle group, both the APT20TTMG (at the dose of 20 mg/kg, i.v., every third day for 3 weeks) and TMZ (at the dose of 5 mg/kg, p.o., daily for 3 weeks) were able to inhibit tumor growth from day 16 maintaining this reduced profile until the end of the assay. It is interesting to note that, on day 20, APT20TTMG reduced the tumor by 46.7±10.7% (p=0.0002) and by 42±13.2% (p<0.0001) on day 22 in relation to the negative control, while TMZ reduced only 41.3±11.1% (p=0.0013) and 31.1±13.4% (p=0.0039) on the same days. Despite the absence of statistical difference between the APT20TTMG and TMZ groups (FIG. 34A) in relation volume tumor and final tumor weight (FIG. 34C), these results may indicate that at longer treatment times APT20TTMG could achieve a better treatment profile than the gold standard GBM treatment. It is also important to highlight that the treatment with the APT20TTMG allowed the animals to gain weight throughout the experiment, suggesting that ASMO is safe for the animals. The immunohistochemistry analysis of Ki67 in tumor samples (count of Ki-67 positive tumor cells and Ki67 total area—FIG. 35), a marker of cellular proliferation, did not detect any statistical differences for this marker between the treated groups and non-treated control. On the other hand, the histopathological findings observed by the pathologist indicated that G3 (APT20TTMG; 20 mg/kg, i.v.) and G2 (TMZ; 5 mg/kg, p.o.) groups showed less anaplastic features (lower grade) and cellular atypia, with fewer mitotic figures in number as compared to G1 (Vehicle control group) (Table 8). Analysis of the total scores for these histopathological findings and tumor volume showed that G3 group (APT20TTMG; 20 mg/kg, i.v.) had statistically lower scores than G1 group (vehicle control), suggesting that APT20TTMG treatment resulted in less aggressive tumor features (FIG. 36).

As shown in FIG. 34A-C, tumor fragments from the U-87MG cell line were implanted in the animals and once the tumor size reached ˜100 mm³ animals were randomized into 3 experimental groups (G1, G2, and G3). G1: Vehicle, (G2) Reference Item Temozolomide, and (G3) APT20TTMG for a total of 22 days. (A) The tumor volume of the animals was measured twice/a week. (B) Animals were weighed throughout the experiment and the change in weight gain compared to pre-treatment weight was plotted. (C) After the euthanasia of the animals, the tumors were removed and weighed.

As shown in FIG. 35A-B, graphs present the individual values of protein quantification of tumors from 8 animals. (A) Ki-67 area, (B) Ki-67 positive tumor cells count.

As shown in FIG. 36, the graph presents the total score value obtained from scores given for histopathological findings and tumor volume from 6 to 8 animals in each group.

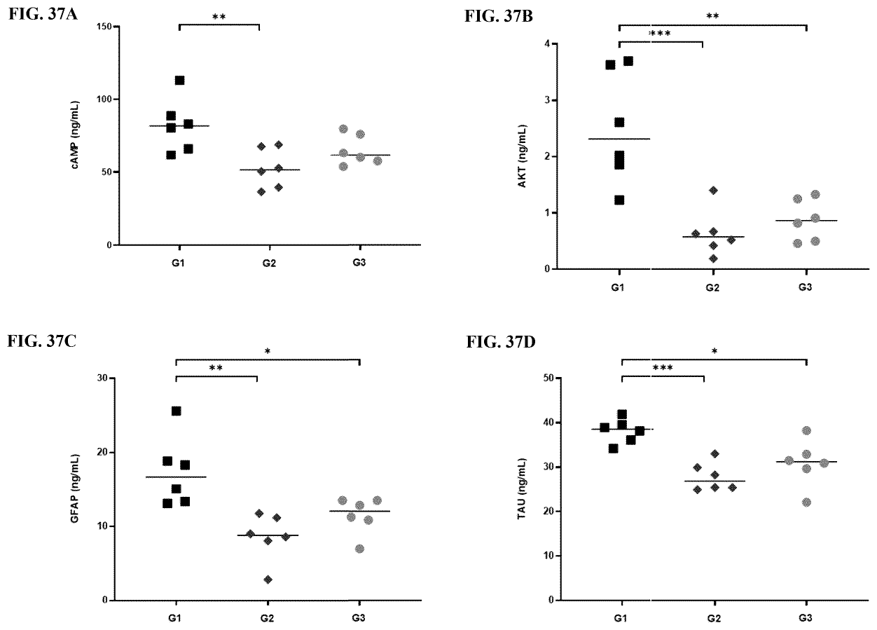

To further understand what are the signaling pathways that were activated by the APT20TTMG in the xenograft mice model of GBM, we have analyzed some important biomarkers (Assessment of cAMP, AKT, GFAP, and TAU) by ELISA assays (FIG. 37) in the U-87MG tumor tissues dissected out from the animals and then homogenized. As the cAMP protein is hypothetically related to a higher dysfunctional splicing activity and a higher ATP production, the assessment of cAMP (FIG. 37A) upon APT20TTMG was carried out but, despite a lower total expression in comparison to the negative control, no statistical difference was observed. The assessment of AKT (FIG. 37B) is interesting since the AKT protein, associated with an upstream PI3K signaling pathway, is responsible for regulating cell survival, proliferation, differentiation, and migration. In a similar way that TMZ, APT20TTMG reduces the expression of ART protein in relation to the negative control, indicating a potential disruption of cancer cell growth and survival mechanisms. Besides ART expression, a high GFAP expression is commonly observed in brain tumors of glial origin, such as astrocytomas and GBMs, being one of the most important markers for astrocyte lineage and reactive astrogliosis and having a pro-tumor effect. Remarkably, the assessment of GFAP (FIG. 37C) shows a reduction of this biomarker upon treatment with APT20TTMG in relation to the non-treated control, indicating an antitumoral potential also through a possible decrease in astrocyte reactivity. Finally, similarly to what was observed in other cell lines, Assessment of TAU (FIG. 37D) shows a significant reduction in TAU expression after APT20TTMG treatment, which could indicate, in the context of GBM, a key interference in cancer migration and progression according to recent studies.

As shown in FIG. 37A-D, the graphs present the individual values of protein quantification of tumors from 6 animals. (A) cAMP, (B) ART, (C) GFAP, and (D) TAU.

TABLE 8
Histopathology findings observed for the tumor samples

| Sample (Group | | |
|---|---|---|
| and animal | | |
| number) | Histopathological findings | Score |
| G1A1 | Capsule - Absent | NSA |
| | Cell Type - Irregular/spindle type cells with presence of | NSA |
| | mixed type of inflammatory cells | |
| | Connective tissue stroma - Moderate Fibrosis | 3 |
| | Apoptosis - Mild | 3 |
| | Lipid content - Minimal | 1 |
| | Necrosis - Prominent | 4 |
| | N:C ratio - Low | 1 |
| | Nuclei - Hyperchromatic | NSA |
| | Mitotic figures - Active (Few in number) | 1 |
| | Grade- 2 | 2 |
| | Volume 1457.7 | 3 |
| | Moderately differentiated | NSA |
| G1A2 | Capsule - Absent | NSA |
| | Cell Type - Spindle type cells infiltrating surrounding | NSA |
| | tissue | |
| | Connective tissue stroma - Mild Fibrosis | 2 |
| | Apoptosis - Mild | 3 |
| | Lipid content - Minimal | 1 |
| | Necrosis - Prominent | 4 |
| | N:C ratio - Low to High | 2 |
| | Nuclei - Hyperchromatic | NSA |
| | Mitotic figures - Active (Large in number) | 3 |
| | Grade- 2 | 2 |
| | Volume 1426.2 | 3 |
| | Moderately differentiated | NSA |
| G1A3 | Capsule - Present | NSA |
| | Cell Type - Cellular atypia and pleomorphism features | NSA |
| | including variably shaped (usually round/oval to | |
| | fusiform type) nuclei with presence of extensive mixed | |
| | infiltrate. | |
| | Connective tissue stroma - Moderate Fibrosis | 3 |
| | Apoptosis - Mild | 3 |
| | Lipid content - Moderate | 3 |
| | Necrosis - Prominent | 4 |
| | N:C ratio - High | 3 |
| | Nuclei - Hyperchromatic | NSA |
| | Mitotic figures - Active (Large in number) | 3 |
| | Grade- 3 | 3 |
| | Volume 1167.5 | 3 |
| | Moderately differentiated | NSA |
| G1A4 | Capsule - Absent | NSA |
| | Cell Type - Irregular/Spindle type cells arranged in | NSA |
| | sheets with presence of mixed type of inflammatory | |
| | cells. | |
| | Connective tissue stroma - Moderate Fibrosis | 3 |
| | Apoptosis - Mild | 3 |
| | Lipid content - Mild | 2 |
| | Necrosis - Prominent | 4 |
| | N:C ratio - Low to High (Intermediate) | 2 |
| | Nuclei - Hyperchromatic | |
| | Mitotic figures - Active (Few in number) | 1 |
| | Grade- 2 | 2 |
| | Volume 1126.7 | 3 |
| | Moderately differentiated | NSA |
| G1A5 | Capsule - Absent | NSA |
| | Cell Type - Irregular/Spindle type cells arranged in | NSA |
| | sheets with scanty basophilic cytoplasm. | |
| | Connective tissue stroma - Moderate Fibrosis | 3 |
| | Apoptosis - Mild | 3 |
| | Lipid content - Moderate | 3 |
| | Necrosis - Moderate | 3 |
| | N:C ratio - High | 3 |
| | Nuclei - Hyperchromatic | |
| | Mitotic figures - Active (Few in number) | 1 |
| | Grade- 2 | 2 |
| | Volume 1272.5 | 3 |
| | Moderately differentiated | NSA |
| G1A6 | Capsule - Present | NSA |
| | Cell Type - Polymorphic (i.e., Moderately differentiated | NSA |
| | to anaplastic) features including variably shaped | |
| | (usually round to fusiform) nuclei, indistinct cell borders | |
| | with mixed type of inflammatory cells. | |
| | Connective tissue stroma - Moderate Fibrosis | 3 |
| | Apoptosis - Mild | 3 |
| | Lipid content - Moderate | 3 |
| | Necrosis - Prominent | 4 |
| | N:C ratio - Low to High (Intermediate) | 2 |
| | Nuclei - Hyperchromatic | NSA |
| | Mitotic figures - Active (Large in number) | 3 |
| | Grade- 3 | 3 |
| | Volume 2585.1 | 5 |
| | Moderately differentiated | NSA |
| G1A7 | Capsule - Present | NSA |
| | Cell Type - Cellular atypia and pleomorphism features | |
| | including variably shaped (usually round/oval to | |
| | fusiform type) nuclei with indistinct cell borders with | |
| | extensive mixed infiltrate. | |
| | Connective tissue stroma - Moderate Fibrosis | 3 |
| | Lipid content - Mild | 2 |
| | Necrosis - Prominent | 4 |
| | N:C ratio - High | 3 |
| | Nuclei - Hyperchromatic | NSA |
| | Mitotic figures - Active (Large in number) | 3 |
| | Grade- 3 | 3 |
| | Volume 1551.2 | 4 |
| | Moderately differentiated | NSA |
| G1A8 | Capsule - Absent | NSA |
| | Cell Type - Spindle type cells infiltrating surrounding | NSA |
| | tissue arranged in sheets | |
| | Connective tissue stroma - Mild Fibrosis | 2 |
| | Apoptosis - Mild | 3 |
| | Lipid content - Minimal | 1 |
| | Necrosis - Prominent | 4 |
| | N:C ratio - Low to High | 2 |
| | Nuclei - Hyperchromatic | NSA |
| | Mitotic figures - Active (Large in number) | 3 |
| | Grade- 3 | 3 |
| | Volume 1051.2 | 3 |
| | Moderately differentiated | NSA |
| G2A1 | Capsule - Present | NSA |
| | Cell Type - Cellular atypia including variably shaped | NSA |
| | (usually round to fusiform) nuclei with scanty | |
| | cytoplasm with mixed type of inflammatory cells. | |
| | Connective tissue stroma - Moderate Fibrosis | 3 |
| | Apoptosis - Mild | 3 |
| | Lipid content - Moderate | 3 |
| | Necrosis - Prominent | 4 |
| | N:C ratio - Low to High (Intermediate) | 2 |
| | Nuclei - Hyperchromatic | NSA |
| | Mitotic figures - Active (Large in number) | 3 |
| | Grade- 2 | 2 |
| | Volume 1593.8 | 4 |
| | Moderately differentiated | NSA |
| G2A2 | Capsule - Present | NSA |
| | Cell Type -Variably shaped (usually spindle/oval to | NSA |
| | fusiform) nuclei with scanty cytoplasm. | |
| | Connective tissue stroma - Moderate Fibrosis | 3 |
| | Apoptosis - Mild | 3 |
| | Lipid content - Moderate | 3 |
| | Necrosis - Prominent | 4 |
| | N:C ratio - Low to High (Intermediate) | 2 |
| | Nuclei - Hyperchromatic | NSA |
| | Mitotic figures - Active (Few in number) | 1 |
| | Grade- 2 | 2 |
| | Volume 113.1 | 1 |
| | Moderately differentiated | NSA |
| G2A3 | Capsule - Present | NSA |
| | Cell Type - Irregular/spindle type cells | NSA |
| | Connective tissue stroma - Mild Fibrosis | 2 |
| | Apoptosis - Mild | 3 |
| | Lipid content - Mild | 2 |
| | Necrosis - Prominent | 4 |
| | N:C ratio - Low to High | 2 |
| | Nuclei - Hyperchromatic | NSA |
| | Mitotic figures - Active (Few in number) | 1 |
| | Grade- 2 | 2 |
| | Volume 937.9 | 2 |
| | Moderately differentiated | NSA |
| G2A4 | Capsule - Absent | NSA |
| | Cell Type - Variably shaped (usually spindle/oval to | NSA |
| | fusiform) nuclei with scanty cytoplasm arranged in | |
| | rosettes (i.e., a halo of cells surrounding | |
| | Connective tissue stroma - Mild Fibrosis | 2 |
| | Apoptosis - Mild | 3 |
| | Lipid content - Moderate | 3 |
| | Necrosis - Prominent | 4 |
| | N:C ratio - Low to High (Intermediate) | 2 |
| | Nuclei - Hyperchromatic | NSA |
| | Mitotic figures - Active (Few in number) | 1 |
| | Grade- 2 | 2 |
| | Volume 339.1 | 1 |
| | Moderately differentiated | NSA |
| G2A5 | Capsule - Present | NSA |
| | Cell Type - Variably shaped (usually spindle/oval or | NSA |
| | irregular) nuclei with basophilic cytoplasm arranged in | |
| | sheets and nest. | |
| | Connective tissue stroma - Mild Fibrosis | 2 |
| | Apoptosis - Mild | 3 |
| | Lipid content - Moderate | 3 |
| | Necrosis - Prominent | 4 |
| | N:C ratio - Low to High (Intermediate) | 2 |
| | Nuclei - Hyperchromatic | NSA |
| | Mitotic figures - Active (Large in number) | 3 |
| | Grade- 3 | 3 |
| | Volume 1447.2 | 3 |
| | Moderately differentiated | NSA |
| G2A6 | Capsule - Present | NSA |
| | Cell Type - Tightly packed irregular/spindle type cells | NSA |
| | with basophilic cytoplasm arranged in sheets and nest | |
| | with extensive mixed type of infiltrate. | |
| | Connective tissue stroma - Mild Fibrosis | 2 |
| | Apoptosis - Moderate | 2 |
| | Lipid content - Minimal | 1 |
| | Necrosis - Moderate | 3 |
| | N:C ratio - Low to High (Intermediate) | 2 |
| | Nuclei - Hyperchromatic | NSA |
| | Mitotic figures - Active (Large in number) | 3 |
| | Grade- 3 | 3 |
| | Volume 830.6 | 2 |
| | Moderately differentiated | NSA |
| G2A7 | Capsule - Absent | NSA |
| | Cell Type - Irregular/spindle type cells | NSA |
| | Connective tissue stroma - Minimal Fibrosis | 1 |
| | Apoptosis - Mild | 3 |
| | Lipid content - Minimal | 1 |
| | Necrosis - Prominent | 4 |
| | N:C ratio - Low to High | 2 |
| | Nuclei - Hyperchromatic | NSA |
| | Mitotic figures - Active (large in number) | 3 |
| | Grade- 3 | 3 |
| | Volume 1277.9 | 3 |
| | Moderately differentiated | NSA |
| G2A8 | Capsule - Present | NSA |
| | Cell Type - Variably shaped (usually spindle/oval or | NSA |
| | fusiform) nuclei with basophilic cytoplasm arranged in | |
| | sheets and nest with extensive mixed type of infiltrate. | |
| | Connective tissue stroma - Mild Fibrosis | 2 |
| | Apoptosis - Mild | 3 |
| | Lipid content - Minimal | 1 |
| | Necrosis - Moderate | 3 |
| | N:C ratio - Low to High (Intermediate) | 2 |
| | Nuclei - Hyperchromatic | NSA |
| | Mitotic figures - Active (Few in number) | 1 |
| | Grade- 2 | 2 |
| | Volume 1479.7 | 3 |
| | Moderately differentiated | NSA |
| G3A1 | Capsule - Present | NSA |
| | Cell Type -Variably shaped (usually spindle/oval to | NSA |
| | fusiform) nuclei with vacuolated cytoplasm arranged in | |
| | sheets and nest. | |
| | Connective tissue stroma - Moderate Fibrosis | 3 |
| | Apoptosis - Mild | 3 |
| | Lipid content - Moderate | 3 |
| | Necrosis - Prominent | 4 |
| | N:C ratio - Low | 1 |
| | Nuclei - Hyperchromatic | NSA |
| | Mitotic figures - Active (Few in number) | 1 |
| | Grade- 2 | 2 |
| | Volume 321.5 | 1 |
| | Moderately differentiated | NSA |
| G3A2 | Capsule - Absent | NSA |
| | Cell Type - Irregular/spindle type cells arranged in | NSA |
| | sheets | |
| | Connective tissue stroma - Mild Fibrosis | 2 |
| | Apoptosis - Moderate | 2 |
| | Lipid content - Minimal | 1 |
| | Necrosis - Moderate | 3 |
| | N:C ratio - Low to High | 2 |
| | Nuclei - Hyperchromatic | NSA |
| | Mitotic figures - Active (Moderate in number) | 2 |
| | Grade- 2 | 2 |
| | Volume 768.1 | 2 |
| | Moderately differentiated | NSA |
| G3A3 | Only diffused necrotic area was observed. | NSA |
| | Grade- Not Applicable | NSA |
| G3A4 | Capsule - Present | 1 |
| | Cell Type - Irregular/spindle type cells | |
| | Connective tissue stroma - Moderate Fibrosis | 3 |
| | Apoptosis - Mild | 3 |
| | Lipid content - Moderate | 3 |
| | Necrosis - Mild | 2 |
| | N:C ratio - Low | 1 |
| | Nuclei - Hyperchromatic | |
| | Mitotic figures - Active (Few in number) | 1 |
| | Grade- 1 | 1 |
| | Volume 796.8 | 2 |
| | Well differentiated | |
| G3A5 | Capsule - Present | 1 |
| | Cell Type - Variably shaped (usually spindle/oval to | NSA |
| | fusiform) nuclei with basophilic cytoplasm arranged in | |
| | sheets and nest with presence of extensive mixed type | |
| | infiltrate. | |
| | Connective tissue stroma - Mild Fibrosis | 2 |
| | Apoptosis - Mild | 3 |
| | Lipid content - Mild | 2 |
| | Necrosis - Prominent | 4 |
| | N:C ratio - High | 3 |
| | Nuclei - Hyperchromatic | NSA |
| | Mitotic figures - Active (Few in number) | 1 |
| | Grade- 2 | 2 |
| | Volume 62.3 | 1 |
| | Moderately differentiated | NSA |
| G3A6 | Capsule - Present | NSA |
| | Cell Type - Variably shaped (usually spindle/oval to | NSA |
| | fusiform) nuclei with basophilic cytoplasm arranged in | |
| | sheets and nest. | |
| | Connective tissue stroma - Moderate Fibrosis | 3 |
| | Apoptosis - Moderate | 2 |
| | Lipid content - Mild | 2 |
| | Necrosis - Prominent | 4 |
| | N:C ratio - Low | 1 |
| | Nuclei - Hyperchromatic | NSA |
| | Mitotic figures - Active (Few in number) | 1 |
| | Grade- 2 | 2 |
| | Volume 1441.5 | 3 |
| | Moderately differentiated | NSA |
| G3A7 | Only diffused necrotic area was observed. | NSA |
| | Grade- Not Applicable | NSA |
| G3A8 | Capsule - Present | NSA |
| | Cell Type - Polymorphic (i.e., Moderately differentiated | NSA |
| | to anaplastic) features including variably shaped | |
| | (usually oval/spindle to fusiform shaped) nuclei with | |
| | presence of mixed type of inflammatory cells | |
| | Connective tissue stroma - Moderate Fibrosis | 3 |
| | Apoptosis - Mild | 3 |
| | Lipid content - Moderate | 3 |
| | Necrosis - Prominent | 4 |
| | N:C ratio - Low to High (Intermediate) | 2 |
| | Nuclei - Hyperchromatic | NSA |
| | Mitotic figures - Active (Large in number) | 3 |
| | Grade- 3 | 3 |
| | Volume 953.5 | 2 |
| | Moderately differentiated | NSA |

Abbreviations: NSA, no score assigned; N:C, nucleus-to-cytoplasmic.

This in vivo study showed that APT20TTMG significantly inhibits tumor growth (tumor volume) in a human glioblastoma (GBM) cancer xenograft model in athymic nude mice, compared to the vehicle group and the gold standard treatment, TMZ. Notably, on days 20 and 22, APT20TTMG showed a more pronounced reduction in tumor size than TMZ. Despite the apparent superior efficacy of APT20TTMG in reducing tumor size, there was no statistical difference between APT20TTMG and TMZ groups in terms of final tumor weight and the vehicle group. On the other hand, the evaluation of histopathological findings and tumor volume as scores indicates that APT20TTMG treatment resulted in a less aggressive tumor. Besides, proteins involved with important signaling pathways in GBM (AKT, GFAP, and TAU) showed a decreased expression upon APT20TTMG treatment, suggesting potential reductions in reactive astrogliosis and disruption of cancer cell growth, migration, progression, and survival mechanism. All these results corroborate with each other and give a better understanding of the pathways related to the observed tumor volume reduction in the in vivo model treated with APT20TTMG. Additionally, APT20TTMG treatment presented potential advantages over TMZ in tumor growth inhibition and was associated with weight gain in the animals, indicating a favorable safety profile. These findings suggest that APT20TTMG can be used an effective therapeutic option for GBM.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1.-132. (canceled)

133. A method of treating cancer in a subject in need thereof, the method comprising administering to the subject a pharmaceutical composition comprising an engineered polynucleotide comprising:
(i) one or more targeting moieties, wherein a targeting moiety of the one or more targeting moieties comprises a nucleic acid sequence complementary to a target sequence, or consensus sequence thereof, of a pre-messenger ribonucleic acid (pre-mRNA); and
(ii) a recruiting moiety for recruitment of a spliceosomal moiety, wherein the recruiting moeity comprises a nucleic acid sequence comprising (i) one or more nucleotides complementary to a sequence of a portion of a spliceosomal moiety or (ii) one or more chemically modified nucleotides,
wherein, when associated with said pre-mRNA and said engineered polynucleotide, said spliceosomal moiety alters said pre-mRNA in or in proximity to said target sequence.

134. The method of claim 133, wherein the cancer is selected from the group consisting of: brain cancer, prostate cancer, breast cancer, renal cancer, kidney cancer, lung cancer, liver cancer, skin cancer, pancreatic cancer, and bone cancer.

135. The method of claim 133, wherein the cancer is a glioblastoma.

136. The method of claim 133, wherein the method reduces the tumor volume ratio or reduces the tumor progression.

137. The method of claim 133, wherein the method: (i) alters the expression of tau; (ii) reduces the expression of TAU; (iii) reduces the total amount of TAU in the subject; (iv) reduces the expression of AKT; or (v) reduces the expression of glial fibrillary acidic protein (GFAP).

138. The method of claim 133, wherein the engineered polynucleotide is administered intravenously.

139. The method of claim 133, wherein the engineered polynucleotide is administered intrathecally.

140. The method of claim 133, wherein the engineered polynucleotide is administered via subcutaneous injection, intravenous injection, intramuscular injection, intradermal injection, transdermal injection percutaneous administration, intranasal administration, intralymphatic injection, intrathecal administration, pulmonary administration, rectal administration intragastric administration, or any other suitable parenteral administration.

141. The method of claim 133, wherein the method (i) decreases premature polyadenylation of one or more transcripts of the subject or (ii) decreases cryptic splicing of one or more transcripts of the subject.

142. The method of claim 133, wherein the plurality of cells comprise a tumor cell and wherein the method: (i) improves a score associated with a histopathological finding, where said finding comprises tumor grade, lipid content, necrosis, or nucleus-to-cytoplasmic (N:C) ratio; (ii) reduces the viability or decreases the proliferation rate of the cell; (iii) increases cell necrosis, cell apoptosis, or increases the number of cells in a necrotic or apoptotic phase; or (iv) alters distribution of cell cycles phases, increases the propensity of the cell to be in a G2/M phase, or increases the number of cells in a G2/M phase.

143. The method of claim 142, wherein the tumor cell comprises a glioma, astrocytoma, neuroblastoma, or carcinoma.

144. The method of claim 133, wherein the method modulates the formation of U1 snRNP complexes.

145. The method of claim 133, wherein said target sequence comprises a splice site.

146. The method of claim 145, wherein said splice site comprises 5′-GU-3′

147. The method of claim 133, wherein said method alters an expression or activity of a target gene, wherein said target gene comprise said target sequence.

148. The method of claim 133, wherein said one or more targeting moieties comprise (1) a first targeting moiety complementary or identical to a first targeted sequence in said target sequence of said pre-mRNA, and (2) a second targeting moiety complementary or identical to a second targeted sequence in said target sequence of said pre-mRNA.

149. The method of claim 148, wherein said first and second targeted sequences are apart in said target sequence by a spacing sequence of no more than five nucleotides (e.g., one or two nucleotides).

150. The method of claim 148, wherein (i) said first targeting moiety comprises a sequence identical or complementary to a sequence selected from the exon sequence column of Table 1; and wherein said second targeting moiety comprises a sequence identical or complementary to a sequence set forth in the intron sequence column of Table 1 or (ii) said first targeting moiety comprises a sequence identical or complementary to a sequence set forth in the intron sequence column of Table 1; and wherein said second targeting moiety comprises a sequence identical or complementary to a sequence set forth in the exon sequence column of Table 1.

151. The method of claim 133, wherein said first targeting moiety comprises a sequence at least 90% identical or complementary to a binding site of a spliceosome snRNA.

152. The method of claim 133, wherein said spliceosomal moiety is selected from a spliceosomal ribonucleoprotein complex, a spliceosomal small nuclear ribonucleic acid (snRNA), a spliceosomal protein, a functional variant thereof, or a functional fragment thereof.

153. The method of claim 152, wherein (i) said spliceosomal snRNA is selected from U1, U2, U4, U5, U6, U11, U12, U14atac, U6atac, and combinations thereof or (ii) wherein said spliceosomal protein is selected from Sm, U1-70k, U1A, U1C, and combinations thereof.

154. The method of claim 133, wherein said recruiting moiety comprises a nucleotide sequence that is at least 90% identical or complementary to any one of SEQ ID Nos: 1 or 2.

155. The method of claim 133, wherein said engineered polynucleotide comprises an apical loop, an upper stem, an internal loop, a lower stem, or a combination thereof

156. The method of claim 133, wherein, when associated with said engineered polynucleotide and said spliceosomal moiety, said pre-mRNA exhibits substantially no base pairing with an RNA binding domain (RBD) of U1 snRNA.

157. The method of claim 133, wherein said engineered polynucleotide is configured to specifically interact with zinc-finger of U1-C protein.

158. The method of claim 133, wherein aid recruiting moiety comprises (i) a nucleotide sequence comprising a phosphorothioate internucleotide linkage that binds to a U1-C zinc finger, or (ii) a nucleotide sequence complementary to a partial sequence of Stem-Loop II (SL2) of U1 snRNA.

159. The method of claim 133, wherein one or more nucleotides of said engineered polynucleotide are 2′-modified nucleotides.

160. The method of claim 159, wherein said 2′-modified nucleotide comprises a 2′-methoxy, 2′-methoxymethyl, 2′-methoxyethyl, 2′ fluoro, or 2′-aminoethyl nucleotide.

161. The method of claim 133, said engineered polynucleotide comprises nucleotides connected by internucleotide linkages and at least one of said internucleotide linkages does not comprise a phosphate.

162. The method of claim 161, wherein said internucleotide linkages comprises a methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino.

163. The method of claim 133, wherein said engineered polynucleotide comprises a nucleotide sequence that is at least 70%, 80%, 85%, or 90% identical or complementary to any one of SEQ ID NOs: 1-4.

164. The method of claim 133, wherein said engineered polynucleotide comprises:
(i) a first targeting moiety configured to specifically bind a pre-messenger ribonucleic acid (pre-mRNA) at a first targeted sequence therein, wherein the first targeting moiety comprises a sequence identical or complementary to 5′-GTCCA-3′,
(ii) a recruiting moiety comprising a sequence that is at least 90% similar or complementary to SEQ ID NO. 1 and is configured to recruit a spliceosomal moiety that comprises U1 snRNA and a U1-C protein, wherein said recruiting moiety comprises an apical loop, an upper stem adjacent to said apical loop, a lower stem, and an internal loop situated between said upper stem and said lower stem, and
(iii) a second targeting moiety configured to specifically bind the pre-mRNA at a second targeted sequence therein, wherein the second targeting moiety comprises a sequence identical or complementary to 5′-CG-3′.
